(19) United States
(12) Patent Application Publication
(10) Pub. No.: US 2023/0382894 A1
(43) Pub. Date: Nov. 30, 2023

(54) CCR4 ANTAGONISTS

(71) Applicant: CHEMOCENTRYX, INC., Thousand Oaks, CA (US)

(72) Inventors: Pingchen FAN, Hayward, CA (US); Rebecca M. LUI, Mountain View, CA (US); Ryan J. SCAMP, Fremont, CA (US); Ju YANG, Palo Alto, CA (US); Yu WANG, Redwood City, CA (US); Yibin ZENG, Foster City, CA (US); Penglie ZHANG, Foster City, CA (US)

(21) Appl. No.: 18/302,041

(22) Filed: Apr. 18, 2023

Related U.S. Application Data
(60) Provisional application No. 63/332,331, filed on Apr. 19, 2022.
(60) Provisional application No. 63/380,126, filed on Oct. 19, 2022.

(51) Int. Cl. C07D 403/04; C07D 403/14; C07D 401/14; C07D 413/14; C07D 405/14

(52) U.S. Cl. CPC C07D 403/04; C07D 403/14; C07D 401/14; C07D 413/14; C07D 405/14

(57) ABSTRACT

Compounds are provided having formula (I):

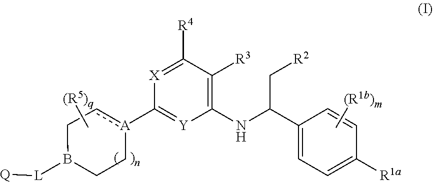

wherein the groups/letters R^{1a}, R^{1b}, m, R², R³, R⁴, X, Y, A, B, n, q, L and Q, have the meanings provided herein. The compounds are useful as CCR4 antagonists and are useful in treated diseases and conditions modulated by CCR4 activity.

### CROSS-REFERENCES TO RELATED APPLICATIONS

This application claims the benefit of priority under 35 U.S.C § 119(e) to U.S. Provisional Application Ser. No. 63/332,331 filed Apr. 19, 2022 and 63/380,126 filed Oct. 19, 2022, the disclosure of each are incorporated herein by reference in their entirety.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Chemokines are chemotactic cytokines that are released by a wide variety of cells to attract macrophages, T cells, eosinophils, basophils and neutrophils to sites of inflammation (reviewed in Schall, Cytokine, 3:165-183 (1991), Schall, et al., Curr. Opin. Immunol. 6:865-873 (1994) and Murphy, Rev. Immun., 12:593-633 (1994)). In addition to stimulating chemotaxis, other changes can be selectively induced by chemokines in responsive cells, including changes in cell shape, transient rises in the concentration of intracellular free calcium ions ([Ca2+]), granule exocytosis, integrin upregulation, formation of bioactive lipids (e.g., leukotrienes) and respiratory burst, associated with leukocyte activation. Thus, the chemokines are early triggers of the inflammatory response, causing inflammatory mediator release, chemotaxis and extravasation to sites of infection or inflammation.

There are two main classes of chemokines, CXC (alpha) and CC (beta), depending on whether the first two cysteines are separated by a single amino acid (C-X-C) or are adjacent (C-C). The alpha-chemokines, such as interleukin-8 (IL-8), neutrophil-activating protein-2 (NAP-2) and melanoma growth stimulatory activity protein (MGSA) are chemotactic primarily for neutrophils, whereas beta-chemokines, such as RANTES, MIP-1a, MIP-1b, monocyte chemotactic protein-1 (MCP-1), MCP-2, MCP-3 and eotaxin are chemotactic for macrophages, T-cells, eosinophils and basophils (Deng, et al., Nature, 381:661-666 (1996)). The chemokines bind specific cell-surface receptors belonging to the family of G-protein-coupled seven-transmembrane-domain proteins (reviewed in Horuk, Trends Pharm. Sci., 15:159-165 (1994)) which are termed “chemokine receptors.”

On binding their cognate ligands, chemokine receptors transduce an intracellular signal though the associated trimeric G protein, resulting in a rapid increase in intracellular calcium concentration. There are at least eleven human chemokine receptors that bind or respond to beta-chemokines and at least seven human chemokine receptors that bind to the alpha chemokines. Additionally CX3CR1 (fractalkine receptor) can bind to the fractalkine chemokine, which is distinguished by a series of three amino acids between the first two cysteines. Chemokine receptors, have been implicated as being important mediators of inflammatory and immunoregulatory disorders and diseases, including asthma and allergic diseases, as well as autoimmune pathologies such as rheumatoid arthritis and atherosclerosis.

The CC Chemokine receptor 4, CCR4, first identified by Power et al. (Power et al. (1995) J. Biol. Chem. 270:19495-19500), is a G protein-coupled receptor that binds to chemokines including CCL22, also known as Macrophage-Derived Chemokine (MDC; a CC chemokine reported to be a chemoattractant for the Th2 subset of peripheral blood T cells, dendritic cells, and natural killer (NK) cells), and CCL17, also known as TARC (thymus and activation-regulated chemokine), which is also produced by monocytes and dendritic cells.

The full-length human CCR4 protein (GenBank Accession No. X85740; SWISS-PROT Accession No. P51679) has been described, see, e.g, Imai et al. (1998) J. Biol. Chem. 273:1764-1768, and has the sequence shown in SEQ ID NO:1.

While the global distribution of CCR4 is unknown, the receptor is expressed primarily in peripheral blood T lymphocytes, and is found on approximately 20% of adult peripheral blood effector/memory CD4+ T cells. CCR4 is involved in T lymphocyte homing to the skin and lungs (see, e.g., Campbell et al. (1999) Nature 400:776-780, Gonzalo et al. (1999) J. Immunol. 163:403-5 411, Lloyd et al. (2000) J. Exp. Med. 191:265-273, Kawasaki et al. (2001) J. Immunol. 166:2055-2062) and is found on almost all T cells that have a skin homing phenotype, the CTLA+ T cells. In fact, CCR4 is highly expressed on cutaneous T cell lymphomas, and targeting such cells with an anti-CCR4 monoclonal antibody has proven efficacious in killing these cancer cells (Reference).

More recently, CCR4 has proven to be an important player in the interactions between cancer cells and the immune system. CCR4 is expressed by many regulatory T cells (Treg) whose dysfunction causes them to prevent effector T cells from destroying cancer cells (Reference). Further, it is likely that CCR4 plays a role in the entry of Treg cells into tumors, and blocking its function will allow effector cells to destroy the tumor.

Thus CCR4 may be an important player in skin pathologies in which leukocytes participate. Its role in Treg trafficking indicates that it is a likely player in immune-oncology. It also seems likely that CCR4 is expressed on some other cell types, probably monocytes/macrophages and dendritic cells, among others. In view of the clinical importance of CCR4, the identification of compounds that modulate CCR4 function represent an attractive avenue into the development of new therapeutic agents. Such compounds and methods for their use are provided herein.

### BRIEF SUMMARY OF THE INVENTION

In the present disclosure, compounds are provided having formula (I):

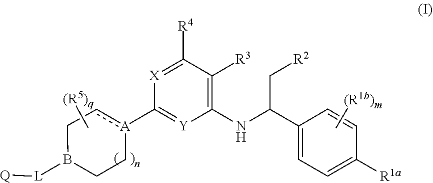

wherein the groups/letters R^{1a}, R^{1b}, m, R², R³, R⁴, X, Y, A, B, n, q, L and Q, have the meanings provided in the Detailed Description.

Compositions containing the compounds of formula (I) are provided as well as methods for treating diseases and conditions modulated by CCR4 activity. Still further, the compounds provided herein are useful in methods of screening for additional CCR4-modulatory compounds.

### BRIEF DESCRIPTION OF THE DRAWINGS

Not Applicable.

### DETAILED DESCRIPTION OF THE INVENTION

While various embodiments and aspects of the present disclosure(s) are shown and described herein, it will be obvious to those skilled in the art that such embodiments and aspects are provided by way of example only. Numerous variations, changes, and substitutions will now occur to those skilled in the art without departing from the disclosure(s). It should be understood that various alternatives to the embodiments of the disclosure(s) described herein may be employed in practicing the disclosure(s).

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The terms “a” or “an,” as used in herein means one or more.

The terms “comprise,” “include,” and “have,” and the derivatives thereof, are used herein interchangeably as comprehensive, open-ended terms. For example, use of “comprising,” “including,” or “having” means that whatever element is comprised, had, or included, is not the only element encompassed by the subject of the clause that contains the verb.

The term “alkyl”, by itself or as part of another substituent, means, unless otherwise stated, a straight or branched chain hydrocarbon radical, having the number of carbon atoms designated (i.e. C₁₋₈ means one to eight carbons). Examples of alkyl groups include methyl, ethyl, n-propyl, isopropyl, n-butyl, t-butyl, isobutyl, sec-butyl, n-pentyl, n-hexyl, n-heptyl, n-octyl, and the like. The term “alkenyl” refers to an unsaturated alkyl group having one or more double bonds. Similarly, the term “alkynyl” refers to an unsaturated alkyl group having one or more triple bonds. Examples of such unsaturated alkyl groups include vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers.

The term “cycloalkyl” refers to hydrocarbon rings having the indicated number of ring atoms (e.g., C₃₋₆cycloalkyl) and being fully saturated or having no more than one double bond between ring vertices. “Cycloalkyl” is also meant to refer to bicyclic and polycyclic hydrocarbon rings such as, for example, bicyclo[2.2.1]heptane, bicyclo[2.2.2]octane, etc.

The term “heterocycloalkyl” or “heterocyclyl” refers to a saturated or partially unsaturated monocyclic ring having the indicated number of ring vertices (e.g., a 3- to 7-membered ring) and having from one to five heteroatoms selected from N, O, and S as ring vertices, wherein the nitrogen and sulfur atoms are optionally oxidized, and the nitrogen atom(s) are optionally quaternized. The heterocycloalkyl may be a monocyclic, a bicyclic or a polycylic ring system, and may be a bridged, spirocyclic, or a fused ring system. Non limiting examples of heterocycloalkyl groups include pyrrolidine, imidazolidine, pyrazolidine, butyrolactam, valerolactam, imidazolidinone, hydantoin, dioxolane, phthalimide, piperidine, 1,4-dioxane, morpholine, thiomorpholine, thiomorpholine-S-oxide, thiomorpholine-S,S-oxide, piperazine, pyran, pyridone, 3-pyrroline, thiopyran, pyrone, tetrahydrofuran, tetrhydrothiophene, quinuclidine, and the like. A heterocycloalkyl group can be attached to the remainder of the molecule through a ring carbon or a heteroatom. Partially unsaturated heterocycloalkyl groups have one or more double bonds in the ring, but heterocycloalkyl group are not aromatic.

The term “alkylene” by itself or as part of another substituent means a divalent radical derived from an alkane, as exemplified by —CH₂CH₂CH₂CH₂—. Typically, an alkyl (or alkylene) group will have from 1 to 24 carbon atoms, and in other embodiments, those groups will have 10 or fewer carbon atoms in the present disclosure. A “lower alkyl” or “lower alkylene” is a shorter chain alkyl or alkylene group, generally having four or fewer carbon atoms. Similarly, “alkenylene” and “alkynylene” refer to the unsaturated forms of “alkylene” having double or triple bonds, respectively.

As used herein, a wavy line, “ [symbol] ”, that intersects a single, double or triple bond in any chemical structure depicted herein, represent the point attachment of the single, double, or triple bond to the remainder of the molecule. A bond represented by [symbol] is meant to depict an optional double bond. As such, the symbol refers to either a single bond or a double bond.

The terms “alkoxy,” “alkylamino” and “alkylthio” (or thioalkoxy) are used in their conventional sense, and refer to those alkyl groups attached to the remainder of the molecule via an oxygen atom, an amino group, or a sulfur atom, respectively. Additionally, for dialkylamino groups, the alkyl portions can be the same or different and can also be combined to form a 3-7 membered ring with the nitrogen atom to which each is attached. Accordingly, a group represented as dialkylamino or —NR^{a}R^{b} is meant to include piperidinyl, pyrrolidinyl, morpholinyl, azetidinyl and the like.

The term “di-(C₁₋₄ alkyl)amino-C₁₋₄ alkyl” refers to an amino group bearing two C₁₋₄ alkyl groups that can be the same or different (e.g., methyl, ethyl, propyl, isopropyl, n-butyl, sec-butyl, isobutyl and tert-butyl) and which is attached to the remainder of the molecule through a C₁₋₄ alkyl group (a one to four carbon alkylene linking group). Examples of di-(C₁₋₄ alkyl)amino-C₁₋₄ alkyl groups include dimethylaminomethyl, 2-(ethyl(methyl)amino)ethyl, 3-(dimethylamino)butyl, and the like.

The terms “halo” or “halogen,” by themselves or as part of another substituent, mean, unless otherwise stated, a fluorine, chlorine, bromine, or iodine atom. Additionally, terms such as “haloalkyl,” are meant to include monohaloalkyl and polyhaloalkyl. For example, the term “C₁₋₄haloalkyl” is mean to include trifluoromethyl, 2,2,2-trifluoroethyl, 4-chlorobutyl, 3-bromopropyl, and the like. In a similar manner, the term “haloalkoxy,” is meant to include monohaloalkoxy and polyhaloalkoxy. For example, the term “C₁₋₄haloalkoxy” is meant to include trifluoromethoxy, 2,2,2-trifluoroethoxy, 4-chlorobutoxy, and the like.

The term “hydroxyalkyl” is meant to refer to an alkyl group as defined above, having one or two hydroxyl groups as substituents. For example, the term “C₁₋₆ hydroxyalkyl” is mean to include 2-hydroxyethyl and 2,4-dihydroxybutyl.

The term “and acid isosteres” means, unless otherwise stated, a group which can replace a carboxylic acid, having an acidic functionality and steric and electronic characteristics that provide a level of activity (or other compound characteristic such as solubility) similar to a carboxylic acid. Representative acid isosteres include, hydroxamic acids, sulfonic acids, sulfinic acids, sulfonamides, acyl-sulfonamides, phosphonic acids, phosphinic acids, phosphoric acids, tetrazole, and oxo-oxadiazoles.

As used herein, the term “heteroatom” is meant to include oxygen (O), nitrogen (N), sulfur (S) and silicon (Si).

The term “substituent” is an atom or group of atoms substituted in place of hydrogen atom(s) of the parent molecule. Non-limiting examples of substituents in this disclosure include R¹ and R², and these substituents can be monovalent or divalent substituents. Monovalent substituents are bonded to the parent moiety by replacing one hydrogen atom of the parent moiety through a single bond. The hydrogen atom that a monovalent substituent replaces may be an available hydrogen atom from a carbon or nitrogen atom of the parent moiety. Divalent substituents are bonded to the parent moiety by replacing two available hydrogen atoms of the parent moiety through a double bond. It is understood that substituents described in this disclosure cannot be attached to a parent moiety in a way that would result in an unstable molecule.

The term “pharmaceutically acceptable salts” is meant to include salts of the active compounds which are prepared with relatively nontoxic acids or bases, depending on the particular substituents found on the compounds described herein. When compounds of the present disclosure contain relatively acidic functionalities, base addition salts can be obtained by contacting the neutral form of such compounds with a sufficient amount of the desired base, either neat or in a suitable inert solvent. Examples of salts derived from pharmaceutically-acceptable inorganic bases include aluminum, ammonium, calcium, copper, ferric, ferrous, lithium, magnesium, manganic, manganous, potassium, sodium, zinc and the like. Salts derived from pharmaceutically-acceptable organic bases include salts of primary, secondary and tertiary amines, including substituted amines, cyclic amines, naturally-occurring amines and the like, such as arginine, betaine, caffeine, choline, N,N′-dibenzylethylenediamine, diethylamine, 2-diethylaminoethanol, 2-dimethylaminoethanol, ethanolamine, ethylenediamine, N ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like. When compounds of the present disclosure contain relatively basic functionalities, acid addition salts can be obtained by contacting the neutral form of such compounds with a sufficient amount of the desired acid, either neat or in a suitable inert solvent. Examples of pharmaceutically acceptable acid addition salts include those derived from inorganic acids like hydrochloric, hydrobromic, nitric, carbonic, monohydrogencarbonic, phosphoric, monohydrogenphosphoric, dihydrogenphosphoric, sulfuric, monohydrogensulfuric, hydriodic, or phosphorous acids and the like, as well as the salts derived from relatively nontoxic organic acids like acetic, propionic, isobutyric, malonic, benzoic, succinic, suberic, fumaric, mandelic, phthalic, benzenesulfonic, p-tolylsulfonic, citric, tartaric, methanesulfonic, and the like. Also included are salts of amino acids such as arginate and the like, and salts of organic acids like glucuronic or galactunoric acids and the like (see, for example, Berge, S. M., et al, “Pharmaceutical Salts”, Journal of Pharmaceutical Science, 1977, 66, 1-19). Certain specific compounds of the present disclosure contain both basic and acidic functionalities that allow the compounds to be converted into either base or acid addition salts.

The neutral forms of the compounds may be regenerated by contacting the salt with a base or acid and isolating the parent compound in the conventional manner. The parent form of the compound differs from the various salt forms in certain physical properties, such as solubility in polar solvents, but otherwise the salts are equivalent to the parent form of the compound for the purposes of the present disclosure.

In addition to salt forms, the present disclosure provides compounds which are in a prodrug form. Prodrugs of the compounds described herein are those compounds that readily undergo chemical changes under physiological conditions to provide the compounds of the present disclosure. Additionally, prodrugs can be converted to the compounds of the present disclosure by chemical or biochemical methods in an ex vivo environment. For example, prodrugs can be slowly converted to the compounds of the present disclosure when placed in a transdermal patch reservoir with a suitable enzyme or chemical reagent.

Certain compounds of the present disclosure can exist in unsolvated forms as well as solvated forms, including hydrated forms. In general, the solvated forms are equivalent to unsolvated forms and are intended to be encompassed within the scope of the present disclosure. Certain compounds of the present disclosure may exist in multiple crystalline or amorphous forms. In general, all physical forms are equivalent for the uses contemplated by the present disclosure and are intended to be within the scope of the present disclosure.

Certain compounds of the present disclosure possess asymmetric carbon atoms (optical centers) or double bonds; the stereoisomers, tautomers, racemates, diastereomers, geometric isomers, regioisomers and individual stereoisomers (e.g., separate enantiomers) are all intended to be encompassed within the scope of the present disclosure. The compounds of the present disclosure may also contain unnatural proportions of atomic isotopes at one or more of the atoms that constitute such compounds. Unnatural proportions of an isotope may be defined as ranging from the amount found in nature to an amount consisting of 100% of the atom in question. For example, the compounds may incorporate radioactive isotopes, such as for example tritium (³H), iodine-125 (¹²⁵I) or carbon-14 (¹⁴C), or non-radioactive isotopes, such as deuterium (²H) or carbon-13 (¹³C). Such isotopic variations can provide additional utilities to those described elsewhere with this application. For instance, isotopic variants of the compounds of the disclosure may find additional utility, including but not limited to, as diagnostic and/or imaging reagents, or as cytotoxic/radiotoxic therapeutic agents. Additionally, isotopic variants of the compounds of the disclosure can have altered pharmacokinetic and pharmacodynamic characteristics which can contribute to enhanced safety, tolerability or efficacy during treatment. All isotopic variations of the compounds of the present disclosure, whether radioactive or not, are intended to be encompassed within the scope of the present disclosure.

The “subject” is defined herein to include animals such as mammals, including, but not limited to, primates (e.g., humans), cows, sheep, goats, horses, dogs, cats, rabbits, rats, mice and the like. In other embodiments, the subject is a human.

As used herein, the phrase “CCR4-mediated condition or disease” and related phrases and terms refer to a condition or disease characterized by inappropriate, e.g., less than or greater than normal, CCR4 functional activity. Inappropriate CCR4 functional activity might arise as the result of CCR4 expression in cells which normally do not express CCR4, increased CCR4 expression (leading to, e.g., inflammatory and immunoregulatory disorders and diseases) or decreased CCR4 expression. Inappropriate CCR4 functional activity might also arise as the result of TARC and/or MDC secretion by cells which normally do not secrete TARC and/or MDC, increased TARC and/or MDC expression (leading to, e.g., inflammatory and immunoregulatory disorders and diseases) or decreased T ARC and/or MDC expression. A CCR4-mediated condition or disease may be completely or partially mediated by inappropriate CCR4 functional activity. However, a CCR4-mediated condition or disease is one in which modulation of CCR4 results in some effect on the underlying condition or disease (e.g., a CCR4 antagonist results in some improvement in patient well-being in at least some patients).

The term “therapeutically effective amount” means the amount of the subject compound that will elicit the biological or medical response of a tissue, system, animal or human that is being sought by the researcher, veterinarian, medical doctor or other clinician.

### I. GENERAL

Compounds of the present disclosure can modulate CCR4 function and are useful in the treatment of various inflammatory and immunoregulatory disorders and diseases.

### II. EMBODIMENTS

A. Compounds

In one aspect, provided herein are compound having formula (I)

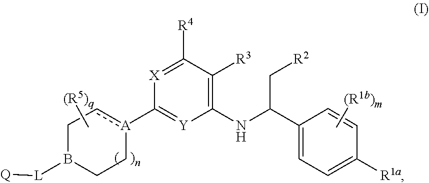

or a pharmaceutically acceptable salt thereof, wherein
R^{1a} is selected from the group consisting of hydrogen, halogen, C₁₋₄ alkyl, C₁₋₄ haloalkyl, —CN, C₁₋₄ alkoxy, and C₁₋₄ haloalkoxy;
m is an integer of from 0 to 4;
each R^{1b} is independently selected from the group consisting of halogen, C₁₋₄ alkyl, C₁₋₄ haloalkyl, —CN, C₁₋₄ alkoxy, and C₁₋₄ haloalkoxy;
R² is selected from the group consisting of H, —OW, —N(R^{a})₂, C₁₋₄ alkyl, C₁₋₄haloalkyl, and C₁₋₄ hydroxyalkyl;
R³ is selected from the group consisting of hydrogen, C₁₋₄ alkyl, halogen, C₁₋₄haloalkyl, C₁₋₄ hydroxyalkyl, C₃₋₈ cycloalkyl, C₁₋₄ alkoxy, C₁₋₄haloalkoxy, C₁₋₄ alkoxy-C₁₋₄ alkyl, —C(O)NH₂, hydroxy, —NH₂, and CN;
each R⁴ is selected from the group consisting of hydrogen, halogen, hydroxy, —CN, C₁₋₄ alkoxy, C₁₋₄haloalkoxy, —NH₂, C₁₋₄ alkyl, C₁₋₄haloalkyl, C₁₋₄ hydroxyalkyl, C₃₋₈ cycloalkyl, —SO₂Me, and —C(O)NH₂;
n is an integer of from 0-2;
X and Y are each independently N or C(R⁴), and at least one of X and Y is N;
[symbol] is a single or double bond;
A is C, N or C(R^{5a}), provided that when A is N, [symbol] is a single bond;
B is N or C(R^{5b}), and at least one of A and B is N;
q is an integer of from 0 to 4;
each R⁵ is independently selected from the group consisting of C₁₋₄ alkyl, C₁₋₄ alkoxy, —C(O)OH, halogen, hydroxy, C₁₋₄haloalkyl, and C₁₋₄ hydroxyalkyl, or two R⁵ are combined to form a one or two carbon bridge between non-adjacent ring vertices;
R^{5a} is selected from the group consisting of hydrogen, C₁₋₄ alkyl, C₁₋₄haloalkyl, and C₁₋₄ hydroxyalkyl;
R^{5b} is selected from the group consisting of hydrogen, C₁₋₄ alkyl, and C₁₋₄ hydroxyalkyl;
L is selected from the group consisting of a bond, —O—, —C(O)—C₀₋₄ alkylene-, —C₁₋₄ alkylene- —C(O), —C(O)N(R^{a})—C₀₋₄ alkylene-, —S(O)₂—C₀₋₄ alkylene and —N(R^{a})C(O)—C₀₋₄ alkylene-;
Q is a member selected from the group consisting of:
   i) 4- to 7-membered heterocyclyl having from one to three heteroatom ring vertices selected from N, O and S, and which is substituted with 0-4 R^{b};
   ii) C₁₋₈ alkyl which is substituted with 0-3 R^{b}; and
   iii) 7- to 11-membered spirocyclyl having from zero to three heteroatom ring vertices selected from N, O and S, and which is substituted with 0-4 R^{b};
   iv) 5- to 6-membered heteroaryl having from one to three heteroatom ring vertices selected from N, O and S, and which is substituted with 0-4 R^{b};
each R^{a} is independently selected from the group consisting of H and C₁₋₄ alkyl optionally substituted with OH or —C(O)OH; and
each R^{b} is independently selected from the group consisting of hydroxy, halogen, oxo, —C₀₋₄ alkylene-N(R^{a})₂, —CO₂R^{a}, C₁₋₄ alkyl, C₁₋₄ alkoxy, C₁₋₄haloalkyl, C₁₋₄ hydroxyalkyl, C₁₋₄ alkylene-CO₂R^{a}, —C₀₋₄ alkylene-heteroaryl, wherein the heteroaryl has from 5- to 6-ring members and one to four heteroatom ring vertices selected from N, O and S, the heteroaryl is optionally substituted with 1-3 R^{c}, —C₀₋₄ alkylene-C₃₋₈ cycloalkyl optionally substituted with 1-3 R^{c}, —C(O)—C₁₋₄ alkyl, and —C₀₋₄ alkylene-C(O)N(R^{a})₂; and
each R^{c} is independently selected from the group consisting of halogen, C₁₋₄ alkyl, C₁₋₄ haloalkyl, and —C(O)OH.

In another aspect, provided herein are compounds having formula (I):

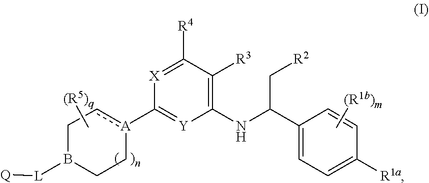

or a pharmaceutically acceptable salt thereof, wherein
R^{1a} is selected from hydrogen, halogen, C₁₋₄ alkyl, C₁₋₄haloalkyl, —CN, C₁₋₄ alkoxy and C₁₋₄ haloalkoxy;
m is an integer of from 0 to 4;
each R^{1b} is independently selected from the group consisting of halogen, C₁₋₄ alkyl, C₁₋₄ haloalkyl, —CN, C₁₋₄ alkoxy and C₁₋₄ haloalkoxy;
R² is selected from the group consisting of H, —OR^{a}, —N(R^{a})₂, C₁₋₄ alkyl, C₁₋₄haloalkyl, and C₁₋₄ hydroxyalkyl;
R³ is selected from the group consisting of halogen, C₁₋₄ alkyl, CN, and CF₃;
each R⁴ is selected from the group consisting of hydrogen, halogen, —CN, C₁₋₄ alkyl, C₁₋₄ haloalkyl, C₁₋₄ hydroxyalkyl, C₃₋₈ cycloalkyl, —SO₂Me and —C(O)NH₂;
n is 0, 1 or 2;
X and Y are each independently N or C(R⁴), and at least one of X and Y is N;
[symbol] is a single or double bond;
A is C, N or C(R^{5a});
B is N or C(R^{5b}), and at least one of A and B is N;
q is an integer of from 0 to 4;
each R⁵ is independently selected from C₁₋₄ alkyl, hydroxy, C₁₋₄ haloalkyl and C₁₋₄ hydroxyalkyl, or two R⁵ are combined to form a one or two carbon bridge between non-adjacent ring vertices;
R^{5a} is selected from the group consisting of hydrogen, C₁₋₄ alkyl, C₁₋₄ haloalkyl and C₁₋₄ hydroxyalkyl;
R^{5b} is hydrogen and C₁₋₄ alkyl;
L is selected from the group consisting of a bond, —C(O)—, —CH₂C(O)—, —C(O)CH₂—, —C(O)N(R^{a})—, and —N(R^{a})C(O)—;
Q is a member selected from the group consisting of:
   i) 4- to 7-membered heterocyclyl having from one to three heteroatom ring vertices selected from N, O and S, and which is substituted with 0-4 R^{b}; and
   ii) C₁₋₈ alkyl which is substituted with 0-3 R^{b};
each R^{a} is independently selected from the group consisting of H and C₁₋₄ alkyl;
each R^{b} is independently selected from the group consisting of hydroxy, oxo, —N(R^{a})₂, —CO₂R^{a}, C₁₋₄ alkyl, C₁₋₄haloalkyl, C₁₋₄ hydroxyalkyl, C₁₋₄ alkylene-CO₂R^{a}, and C₁₋₄ alkylene-N(R^{a})₂.

In some embodiments, the first listed moiety in the L group is attached to the ring comprising variable position B.

In some embodiments, the compound of formula (I) is optically enriched or optically pure.

In one group of embodiments, compounds of formula (I) are provided wherein n is 1.

In another group of embodiments, compounds of formula (I) are provided wherein R^{1a} and R^{1b} are each halogen. In yet another group of embodiments, compounds of formula (I) are provided wherein R² is H or CH₃. In still another group of embodiments, compounds of formula (I) are provided wherein R³ is halogen. In another group of embodiments, compounds of formula (I) are provided wherein each R⁴ is selected from the group consisting of hydrogen, halogen, —CN, C₁₋₄ alkyl, and C₁₋₄ haloalkyl. In another group of embodiments, compounds of formula (I) are provided wherein R⁴ is hydrogen. In yet another group of embodiments, compounds of formula (I) are provided wherein Q is selected from the group consisting of azetidinyl, pyrrolidinyl, piperidinyl and piperazinyl, each of which is substituted with 0-2 R^{b}.

In another group of embodiments, compounds of formula (I) have sub-formula (Ia), or a pharmaceutically acceptable salt thereof:

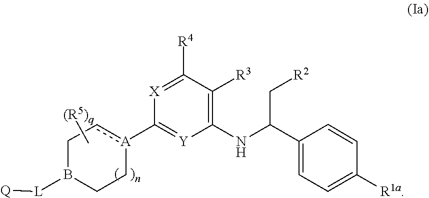

In some selected embodiments, compounds of formula (Ia) are provided wherein Q is selected from the group consisting of azetidinyl, pyrrolidinyl, piperidinyl and piperazinyl, each of which is substituted with 0-2 R^{b}. In other selected embodiments, compounds of formula (Ia) are provided wherein Q is C₁₋₈ alkyl which is substituted with 0-3 R^{b}. Still further selected embodiments are those compounds wherein L is selected from the group consisting of a bond, —C(O)—, —CH₂C(O)—, —C(O)CH₂—, —C(O)NH—, —NHC(O)—, —C(O)N(CH₃)—, and —N(CH₃)C(O)—.

In another group of embodiments, compounds of formula (I) have sub-formula (Ib), or a pharmaceutically acceptable salt thereof:

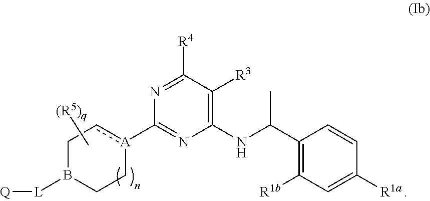

In some selected embodiments, compounds of formula (Ib) are provided wherein Q is selected from the group consisting of azetidinyl, pyrrolidinyl, piperidinyl and piperazinyl, each of which is substituted with 0-2 R^{b}. In other selected embodiments, compounds of formula (Ib) are provided wherein Q is C₁₋₈ alkyl which is substituted with 0-3 R^{b}. Still further selected embodiments are those compounds wherein L is selected from the group consisting of a bond, —C(O)—, —CH₂C(O)—, —C(O)CH₂—, —C(O)NH—, —NHC(O)—, —C(O)N(CH₃)—, and —N(CH₃)C(O)—.

In another group of embodiments, compounds of formula (I) have sub-formula (Ic1), (Ic2), or (Ic3), or a pharmaceutically acceptable salt thereof:

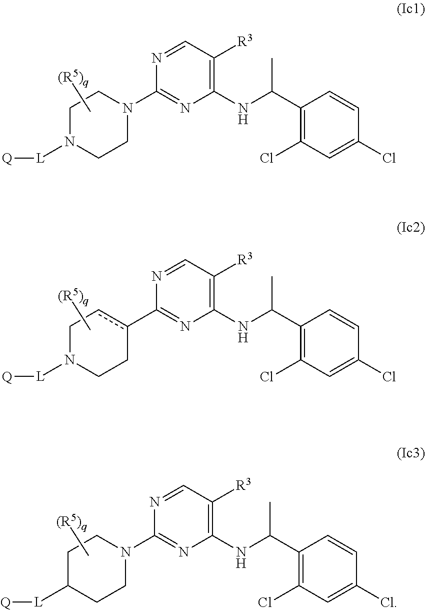

In some selected embodiments, compounds of formula (Ic1), (Ic2), or (Ic3), are provided wherein Q is selected from the group consisting of azetidinyl, pyrrolidinyl, piperidinyl and piperazinyl, each of which is substituted with 0-2 R^{b}. In other selected embodiments, compounds of formula (Ic1), (Ic2), or (Ic3), are provided wherein Q is C₁₋₈ alkyl which is substituted with 0-3 R^{b}. Still further selected embodiments are those compounds wherein L is selected from the group consisting of a bond, —C(O)—, —CH₂C(O)—, —C(O)CH₂—, —C(O)NH—, —NHC(O)—, —C(O)N(CH₃)—, and —N(CH₃)C(O)—.

In another group of embodiments, compounds of formula (I) have sub-formula (Id1), (Id2), or (Id3), or a pharmaceutically acceptable salt thereof:

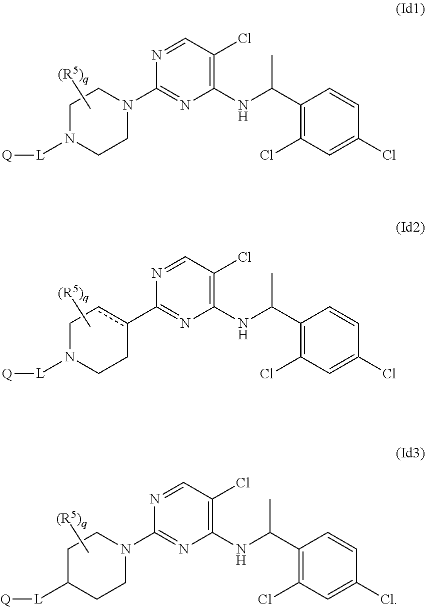

In some selected embodiments, compounds of formula (Id1), (Id2), or (Id3), are provided wherein Q is selected from the group consisting of azetidinyl, pyrrolidinyl, piperidinyl and piperazinyl, each of which is substituted with 0-2 R^{b}. In other selected embodiments, compounds of formula (Id1), (Id2), or (Id3), are provided wherein Q is C₁₋₈ alkyl which is substituted with 0-3 R^{b}. Still further selected embodiments are those compounds wherein L is selected from the group consisting of a bond, —C(O)—, —CH₂C(O)—, —C(O)CH₂—, —C(O)NH—, —NHC(O)—, —C(O)N(CH₃)—, and —N(CH₃)C(O)—.

In some selected embodiments, compounds of formula (Ie1) or a pharmaceutically acceptable salt thereof, are provided:

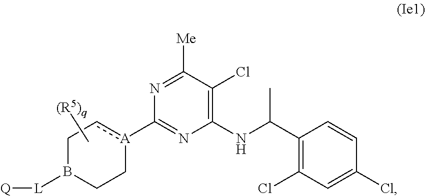

wherein Q, L, B, A, q, and R⁵ have the meanings provided above for formula (I).

In some embodiments, compounds of formula (If1), (If2), (If3) or a pharmaceutically acceptable salt thereof, are provided:

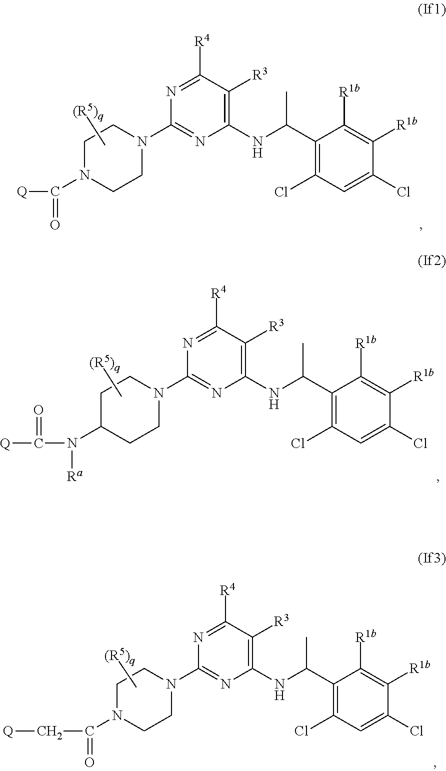

wherein, each R^{b} is independently selected from the group consisting of H, Cl or F, provided that at least one R^{b} is H.

In some selected embodiments, compounds of formula (I), (1a), (Ib), (Ic1), (Ic2), (Ic3), (Id1), (Id2), (Id3), (Ie1), (If1), (If2), (If3), (Ig1) to (Ig10), and (Ih1) to (Ih12) are provided wherein Q is selected from the group consisting of

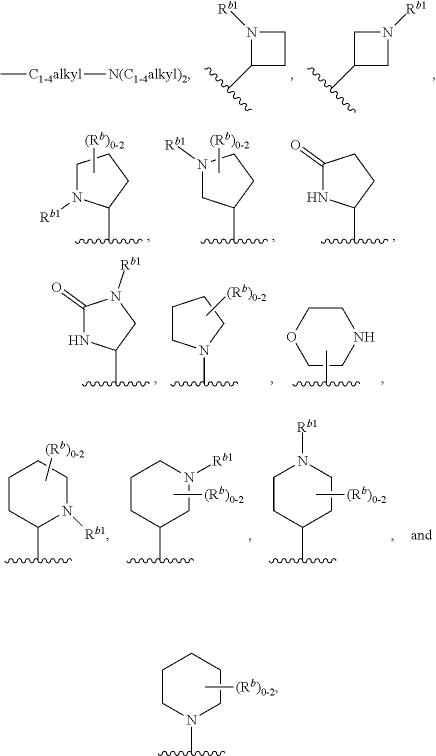

wherein:
each R^{b} is independently selected from the group consisting of C₁₋₄ alkyl, F, Cl, OH, and —N(H)CH₃; and
R^{b1} is selected from the group consisting of H, C₁₋₄ alkyl, C₁₋₄ hydroxyalkyl, C₁₋₄haloalkyl, —C(O)—C₁₋₃ alkyl, —C(O)—O—C₁₋₃ alkyl, —C₁₋₃ alkylene-C(O)OH, and —C(O)NH₂.

In some embodiments, compounds of formula (Ig1) to (Ig10) or a pharmaceutically acceptable salt thereof, are provided:

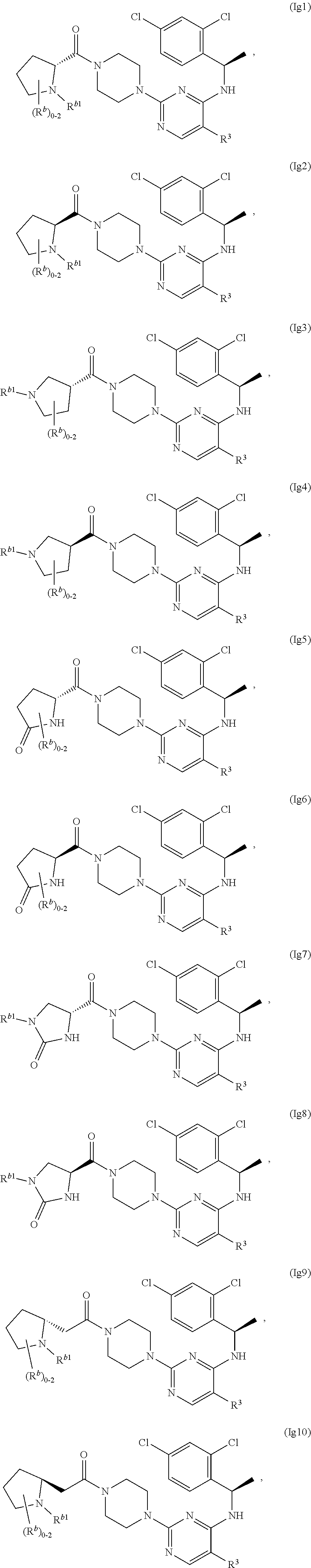

wherein: each R^{b} is independently selected from the group consisting of C₁₋₄ alkyl, F, Cl, OH, and —N(H)CH₃; and
R^{b1} is selected from the group consisting of H, C₁₋₄ alkyl, C₁₋₄ hydroxyalkyl, C₁₋₄haloalkyl, —C(O)—C₁₋₃ alkyl, —C(O)—O—C₁₋₃ alkyl, —C₁₋₃ alkylene-C(O)OH, and —C(O)NH₂.

In some embodiments, compounds of formula (Ih1) to (Ih12) or a pharmaceutically acceptable salt thereof, are provided:

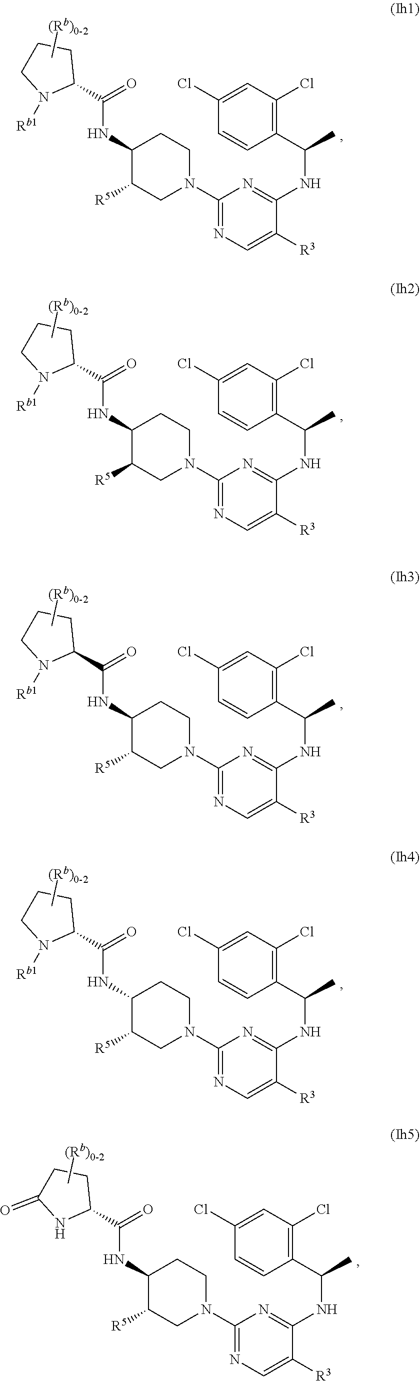

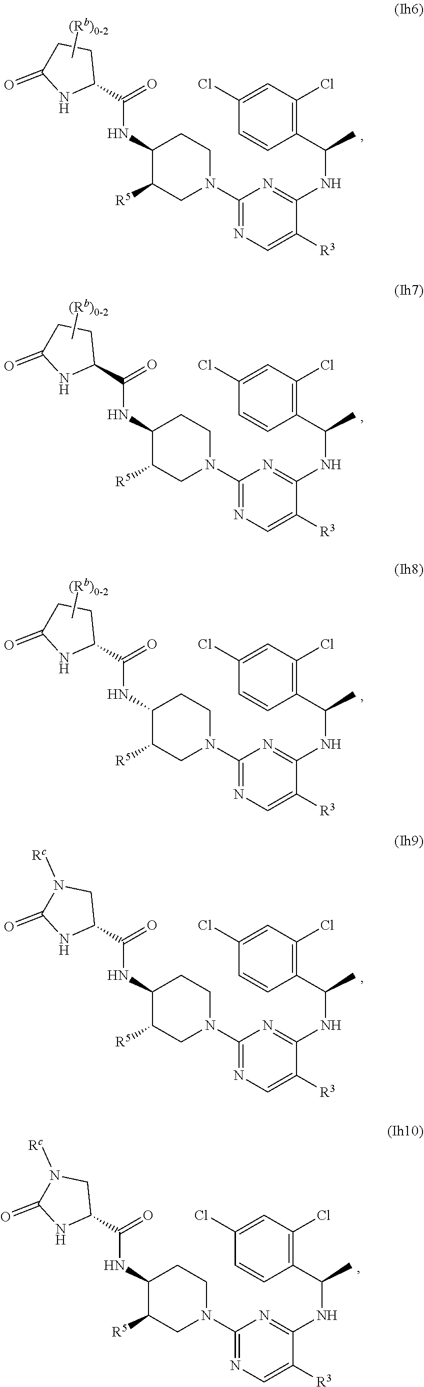

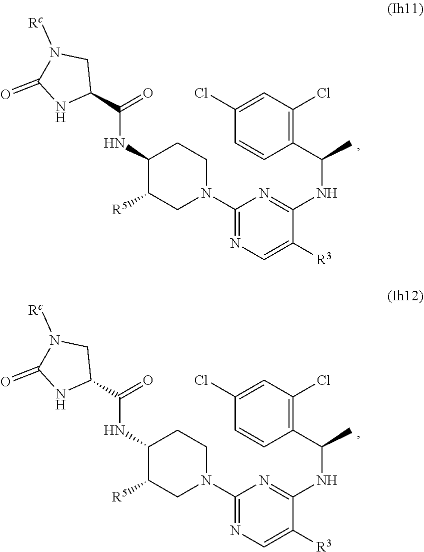

wherein: each R^{b} is independently selected from the group consisting of C₁₋₄ alkyl, F, Cl, and OH; and
R⁵ is selected from the group consisting of OH, F and OCH₃.

In some selected embodiments, compounds of formula (I), (1a), (1b), (Ic1), (Ic2), (Ic3), (Id1), (Id2), (Id3), (Ie1), (If1), (If2), (If3), (Ig1) to (Ig10), and (Ih1) to (Ih12) are provided wherein R³ is selected from the group consisting of C₁₋₄ alkyl, Cl, F, C₁₋₄ haloalkyl, C₁₋₄ hydroxyalkyl, cyclopropyl, C₁₋₄ alkoxy, C₁₋₄haloalkoxy, C₁₋₃ alkylene-O—C₁₋₃ alkyl, hydroxy, —NH₂, and CN. In some embodiments, R³ is selected from the group consisting of C₁, F, —OCH₃, —OCH₂CH₃, OCH(CH₃)₂, OCF₃, OCHF₂, —C(CH₂)₂OH, and hydroxy. In some embodiments, R³ is selected from the group consisting of halogen, —CH₃, —OCH₃, —CH₂OCH₃, —OCH₂CH₃, —OC(H)(CH₃)₂, CN, —NH₂, CF₃, —OCF₃, and —OCHF₂. In some embodiments, R³ is C₁.

In some embodiments, the compounds of formula (I) are selected from the compounds of Table 1, below having ++ or +++ activity.

In some embodiments, the compounds of formula (I) are selected from the compounds of Table 1, below having +++ activity.

B. Compositions

In addition to the compounds provided above, compositions for modulating CCR4 activity in humans and animals will typically contain a pharmaceutical carrier or diluent.

The term “composition” as used herein is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

The pharmaceutical compositions for the administration of the compounds of this disclosure may conveniently be presented in unit dosage form and may be prepared by any of the methods well known in the art of pharmacy and drug delivery. All methods include the step of bringing the active ingredient into association with the carrier which constitutes one or more accessory ingredients. In general, the pharmaceutical compositions are prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired effect upon the process or condition of diseases.

The pharmaceutical compositions containing the active ingredient may be in a form suitable for oral use, for example, as tablets, troches, lozenges, aqueous or oily suspensions, dispersible powders or granules, emulsions and self-emulsifications as described in U.S. Patent Application 2002-0012680, hard or soft capsules, syrups, elixirs, solutions, buccal patch, oral gel, chewing gum, chewable tablets, effervescent powder and effervescent tablets. Compositions intended for oral use may be prepared according to any method known to the art for the manufacture of pharmaceutical compositions and such compositions may contain one or more agents selected from the group consisting of sweetening agents, flavoring agents, coloring agents, antioxidants and preserving agents in order to provide pharmaceutically elegant and palatable preparations. Tablets contain the active ingredient in admixture with non-toxic pharmaceutically acceptable excipients which are suitable for the manufacture of tablets. These excipients may be for example, inert diluents, such as cellulose, silicon dioxide, aluminum oxide, calcium carbonate, sodium carbonate, glucose, mannitol, sorbitol, lactose, calcium phosphate or sodium phosphate; granulating and disintegrating agents, for example, corn starch, or alginic acid; binding agents, for example PVP, cellulose, PEG, starch, gelatin or acacia, and lubricating agents, for example magnesium stearate, stearic acid or talc. The tablets may be uncoated or they may be coated, enterically or otherwise, by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and U.S. Pat. No. 4,265,874 to form osmotic therapeutic tablets for control release.

Formulations for oral use may also be presented as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example peanut oil, liquid paraffin, or olive oil. Additionally, emulsions can be prepared with a non-water miscible ingredient such as oils and stabilized with surfactants such as mono-diglycerides, PEG esters and the like.

Aqueous suspensions contain the active materials in admixture with excipients suitable for the manufacture of aqueous suspensions. Such excipients are suspending agents, for example sodium carboxymethylcellulose, methylcellulose, hydroxy-propylmethylcellulose, sodium alginate, polyvinyl-pyrrolidone, gum tragacanth and gum acacia; dispersing or wetting agents may be a naturally-occurring phosphatide, for example lecithin, or condensation products of an alkylene oxide with fatty acids, for example polyoxy-ethylene stearate, or condensation products of ethylene oxide with long chain aliphatic alcohols, for example heptadecaethyleneoxycetanol, or condensation products of ethylene oxide with partial esters derived from fatty acids and a hexitol such as polyoxyethylene sorbitol monooleate, or condensation products of ethylene oxide with partial esters derived from fatty acids and hexitol anhydrides, for example polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, such as sucrose or saccharin.

Oily suspensions may be formulated by suspending the active ingredient in a vegetable oil, for example arachis oil, olive oil, sesame oil or coconut oil, or in a mineral oil such as liquid paraffin. The oily suspensions may contain a thickening agent, for example beeswax, hard paraffin or cetyl alcohol. Sweetening agents such as those set forth above, and flavoring agents may be added to provide a palatable oral preparation. These compositions may be preserved by the addition of an anti-oxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, for example sweetening, flavoring and coloring agents, may also be present.

The pharmaceutical compositions of the disclosure may also be in the form of oil-in-water emulsions. The oily phase may be a vegetable oil, for example olive oil or arachis oil, or a mineral oil, for example liquid paraffin or mixtures of these. Suitable emulsifying agents may be naturally-occurring gums, for example gum acacia or gum tragacanth, naturally-occurring phosphatides, for example soy bean, lecithin, and esters or partial esters derived from fatty acids and hexitol anhydrides, for example sorbitan monooleate, and condensation products of the said partial esters with ethylene oxide, for example polyoxyethylene sorbitan monooleate. The emulsions may also contain sweetening and flavoring agents.

Syrups and elixirs may be formulated with sweetening agents, for example glycerol, propylene glycol, sorbitol or sucrose. Such formulations may also contain a demulcent, a preservative and flavoring and coloring agents. Oral solutions can be prepared in combination with, for example, cyclodextrin, PEG and surfactants.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent, for example as a solution in 1,3-butane diol. Among the acceptable vehicles and solvents that may be employed are water, Ringer's solution and isotonic sodium chloride solution. In addition, sterile, fixed oils are conventionally employed as a solvent or suspending medium. For this purpose any bland fixed oil may be employed including synthetic mono- or diglycerides. In addition, fatty acids such as oleic acid find use in the preparation of injectables.

The compounds of the present disclosure may also be administered in the form of suppositories for rectal administration of the drug. These compositions can be prepared by mixing the drug with a suitable non-irritating excipient which is solid at ordinary temperatures but liquid at the rectal temperature and will therefore melt in the rectum to release the drug.

Such materials include cocoa butter and polyethylene glycols. Additionally, the compounds can be administered via ocular delivery by means of solutions or ointments. Still further, transdermal delivery of the subject compounds can be accomplished by means of iontophoretic patches and the like. For topical use, creams, ointments, jellies, solutions or suspensions, etc., containing the compounds of the present disclosure are employed. As used herein, topical application is also meant to include the use of mouth washes and gargles.

The compounds of this disclosure may also be coupled a carrier that is a suitable polymers as targetable drug carriers. Such polymers can include polyvinylpyrrolidone, pyran copolymer, polyhydroxy-propyl-methacrylamide-phenol, polyhydroxyethyl-aspartamide-phenol, or polyethyleneoxide-polylysine substituted with palmitoyl residues. Furthermore, the compounds of the disclosure may be coupled to a carrier that is a class of biodegradable polymers useful in achieving controlled release of a drug, for example polylactic acid, polyglycolic acid, copolymers of polylactic and polyglycolic acid, polyepsilon caprolactone, polyhydroxy butyric acid, polyorthoesters, polyacetals, polydihydropyrans, polycyanoacrylates and cross linked or amphipathic block copolymers of hydrogels. Polymers and semipermeable polymer matrices may be formed into shaped articles, such as valves, stents, tubing, prostheses and the like.

In view of the above, provided herein are embodiments of pharmaceutical compositions comprising a pharmaceutically acceptable excipient and a compound of any of formulae (I), (Ia), (Ib), (Ic1), (Ic2), (Ic3), (Id1), (Id2), (Id3), (Ie1), (If1), (If2), (If3), (Ig1) to (Ig10), or (Ih1) to (Ih12), as described above, or a pharmaceutically acceptable salt thereof.

C. Methods of Use

In another aspect, the present disclosure provides methods of treating or preventing a CCR4-mediated condition or disease by administering to a subject having such a condition or disease, a therapeutically effective amount of any compound of Formula I. Compounds for use in the present methods are those compounds provided in the embodiments herein, as well as compounds specifically set forth in the Examples below, in the attached Figures; and provided with specific structures herein.

Diseases and conditions associated with inflammation, infection and cancer can be treated or prevented with the present compounds and compositions. In one group of embodiments, diseases or conditions, including chronic diseases, of humans or other species can be treated with inhibitors of CCR4 function. These diseases or conditions include: (1) allergic diseases such as systemic anaphylaxis or hypersensitivity responses, drug allergies, insect sting allergies and food allergies, (2) inflammatory bowel diseases, such as Crohn's disease, ulcerative colitis, ileitis and enteritis, (3) vaginitis, (4) psoriasis and inflammatory dermatoses such as dermatitis, eczema, atopic dermatitis, allergic contact dermatitis, dermatomyositis, lichen planus, bullous pemphigoid, urticaria and pruritus, (5) vasculitis, (6) spondyloarthropathies, (7) scleroderma, (8) asthma and respiratory allergic diseases such as allergic asthma, exercise-induced asthma, allergic rhinitis, hypersensitivity lung diseases and the like, (9) autoimmune diseases, such as arthritis (including rheumatoid and psoriatic), multiple sclerosis, systemic lupus erythematosus, type I diabetes, glomerulonephritis, and the like, (10) graft rejection (including allograft rejection and graft-v-host disease), and (11) leukemias, lymphomas, and other blood borne cancers including cutaneous T cell lymphoma, mycosis fungoides, acute lymphoblastic leukemias and the like, and (12) other diseases in which undesired inflammatory responses are to be inhibited, such as atherosclerosis, myositis, neurodegenerative diseases (e.g., Alzheimer's disease), encephalitis, meningitis, hepatitis, nephritis, sepsis, sarcoidosis, allergic conjunctivitis, otitis, chronic obstructive pulmonary disease, sinusitis, Behcet's syndrome and gout.

In another group of embodiments, diseases or conditions can be treated with agonists of CCR4 function. Examples of diseases to be treated with CCR4 agonists include cancers, diseases in which angiogenesis or neovascularization play a role (neoplastic diseases, retinopathy and macular degeneration), infectious diseases (viral infections, e.g., HIV infection, and bacterial infections) and immunosuppressive diseases such as organ transplant conditions and skin transplant conditions. The term “organ transplant conditions” is meant to include bone marrow transplant conditions and solid organ (e.g., kidney, liver, lung, heart, pancreas or combination thereof) transplant conditions.

Preferably, the present methods are directed to the treatment of diseases or conditions selected from allergic diseases (including skin allergies and allergic airway disorders), atopic allergic conditions including atopic dermatitis, psoriasis, cancer (including solid tumors and metastatic disease) and asthma. Depending on the disease to be treated and the subject's condition, the compounds of the present disclosure may be administered by oral, parenteral (e.g., intramuscular, intraperitoneal, intravenous, ICV, intracisternal injection or infusion, subcutaneous injection, or implant), inhalation, nasal, vaginal, rectal, sublingual, or topical routes of administration and may be formulated, alone or together, in suitable dosage unit formulations containing conventional non-toxic pharmaceutically acceptable carriers, adjuvants and vehicles appropriate for each route of administration. The present disclosure also contemplates administration of the compounds of the present disclosure in a depot formulation.

Those of skill in the art will understand that agents that modulate CCR4 activity can be combined in treatment regimens with other therapeutic agents and/or with chemotherapeutic agents or radiation. In some cases, the amount of chemotherapeutic agent or radiation is an amount which would be sub-therapeutic if provided without combination with a composition of the disclosure. Those of skill in the art will appreciate that “combinations” can involve combinations in treatments (i.e., two or more drugs can be administered as a mixture, or at least concurrently or at least introduced into a subject at different times but such that both are in the bloodstream of a subject at the same time). Additionally, compositions of the current disclosure may be administered prior to or subsequent to a second therapeutic regimen, for instance prior to or subsequent to a dose of chemotherapy or irradiation.

In the treatment or prevention of conditions which require chemokine receptor modulation an appropriate dosage level will generally be about 0.001 to 100 mg per kg patient body weight per day which can be administered in single or multiple doses. Preferably, the dosage level will be about 0.01 to about 25 mg/kg per day; more preferably about 0.05 to about 10 mg/kg per day. A suitable dosage level may be about 0.01 to 25 mg/kg per day, about 0.05 to 10 mg/kg per day, or about 0.1 to 5 mg/kg per day. Within this range the dosage may be 0.005 to 0.05, 0.05 to 0.5 or 0.5 to 5.0 mg/kg per day. For oral administration, the compositions are preferably provided in the form of tablets containing 1.0 to 1000 milligrams of the active ingredient, particularly 1.0, 5.0, 10.0, 15.0, 20.0, 25.0, 50.0, 75.0, 100.0, 150.0, 200.0, 250.0, 300.0, 400.0, 500.0, 600.0, 750.0, 800.0, 900.0, and 1000.0 milligrams of the active ingredient for the symptomatic adjustment of the dosage to the patient to be treated. The compounds may be administered on a regimen of 1 to 4 times per day, preferably once or twice per day.

It will be understood, however, that the specific dose level and frequency of dosage for any particular patient may be varied and will depend upon a variety of factors including the activity of the specific compound employed, the metabolic stability and length of action of that compound, the age, body weight, hereditary characteristics, general health, sex and diet of the subject, as well as the mode and time of administration, rate of excretion, drug combination, and the severity of the particular condition for the subject undergoing therapy.

In one group of embodiments, the compounds and compositions described herein can be combined with other compounds and compositions having related utilities to prevent and treat cancer and diseases or conditions associated with CCR4 signaling. Such other drugs may be administered, by a route and in an amount commonly used therefor, contemporaneously or sequentially with a compound or composition of the present disclosure. When a compound or composition of the present disclosure is used contemporaneously with one or more other drugs, a pharmaceutical composition containing such other drugs in addition to the compound or composition of the present disclosure. Accordingly, the pharmaceutical compositions of the present disclosure include those that also contain one or more other active ingredients or therapeutic agents, in addition to a compound or composition of the present disclosure. Examples of other therapeutic agents that may be combined with a compound or composition of the present disclosure, either administered separately or in the same pharmaceutical compositions, include, but are not limited to: cisplatin, paclitaxel, methotrexate, cyclophosphamide, ifosfamide, chlorambucil, carmustine, carboplatin, vincristine, vinblastine, thiotepa, lomustine, semustine, 5-fluorouracil, corticosteroids, calcineurin inhibitors, NSAIDs, inhibitors of 5-lipoxygenase, and cytarabine. The weight ratio of the compound of the present disclosure to the second active ingredient may be varied and will depend upon the effective dose of each ingredient. Generally, an effective dose of each will be used. Thus, for example, when a compound of the present disclosure is combined with a second anticancer agent, the weight ratio of the compound of the present disclosure to the second agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used.

Methods of Treating Inflammation

Still further, the compounds and compositions of the present disclosure are useful for the treatment of inflammation, and can be combined with other compounds and compositions having therapeutic utilities that may require treatment either before, after or simultaneously with the treatment of cancer or inflammation with the present compounds. Accordingly, combination methods and compositions are also a component of the present disclosure to prevent and treat the condition or disease of interest, such as inflammatory or autoimmune disorders, conditions and diseases, including psoriasis, dermatomyositis, inflammatory bowel disease, rheumatoid arthritis, osteoarthritis, psoriatic arthritis, polyarticular arthritis, multiple sclerosis, allergic diseases, atopic dermatitis and asthma, and those pathologies noted above.

For example, in the treatment or prevention of inflammation or autoimmunity or for example arthritis associated bone loss, the present compounds and compositions may be used in conjunction with an anti-inflammatory or analgesic agent such as an opiate agonist, a lipoxygenase inhibitor, such as an inhibitor of 5-lipoxygenase, a cyclooxygenase inhibitor, such as a cyclooxygenase-2 inhibitor, an interleukin inhibitor, such as an interleukin-1 inhibitor, an NMDA antagonist, an inhibitor of nitric oxide or an inhibitor of the synthesis of nitric oxide, a non-steroidal anti-inflammatory agent, or a cytokine-suppressing anti-inflammatory agent, for example with a compound such as acetaminophen, aspirin, codeine, fentanyl, ibuprofen, indomethacin, ketorolac, morphine, naproxen, phenacetin, piroxicam, a steroidal analgesic, sufentanyl, sunlindac, tenidap, and the like. Similarly, the instant compounds and compositions may be administered with an analgesic listed above; a potentiator such as caffeine, an H2 antagonist (e.g., ranitidine), simethicone, aluminum or magnesium hydroxide; a decongestant such as phenylephrine, phenylpropanolamine, pseudoephedrine, oxymetazoline, ephinephrine, naphazoline, xylometazoline, propylhexedrine, or levo desoxy ephedrine; an antitussive such as codeine, hydrocodone, caramiphen, carbetapentane, or dextromethorphan; a diuretic; and a sedating or non-sedating antihistamine.

As noted, compounds and compositions of the present disclosure may be used in combination with other drugs that are used in the treatment, prevention, suppression or amelioration of the diseases or conditions for which compounds and compositions of the present disclosure are useful. Such other drugs may be administered, by a route and in an amount commonly used therefor, contemporaneously or sequentially with a compound or composition of the present disclosure. When a compound or composition of the present disclosure is used contemporaneously with one or more other drugs, a pharmaceutical composition containing such other drugs in addition to the compound or composition of the present disclosure is contemplated. Accordingly, the pharmaceutical compositions of the present disclosure include those that also contain one or more other active ingredients or therapeutic agents, in addition to a compound or composition of the present disclosure. Examples of other therapeutic agents that may be combined with a compound or composition of the present disclosure, either administered separately or in the same pharmaceutical compositions, include, but are not limited to: (a) VLA-4 antagonists, (b) corticosteroids, such as beclomethasone, methylprednisolone, betamethasone, prednisone, prenisolone, dexamethasone, fluticasone, hydrocortisone, budesonide, triamcinolone, salmeterol, salmeterol, salbutamol, formeterol; (c) immunosuppressants such as cyclosporine (cyclosporine A, Sandimmune®, Neoral®), tacrolirnus (FK-506, Prograf®), rapamycin (sirolimus, Rapamune®) and other FK-506 type immunosuppressants, and rnycophenolate, e.g., mycophenolate mofetil (CellCept®); (d) antihistamines (H1-histamine antagonists) such as bromopheniramine, chlorpheniramine, dexchloipheniramine, triprolidine, clemastine, diphenhydramine, diphenylpyraline, tripelennamine, hydroxyzine, methdilazine, promethazine, trimeprazine, azatadine, cyproheptadine, antazoline, pheniramine pyrilamine, astemizole, terfenadine, loratadine, cetirizine, fexofenadine, descarboethoxyloratadine, and the like; (e) non-steroidal anti asthmatics (e.g., terbutaline, metaproterenol, fenoterol, isoetharine, albuterol, bitolterol and pirbuterol), theophylline, cromolyn sodium, atropine, ipratropium bromide, leukotriene antagonists (e.g., zafmlukast, montelukast, pranlukast, iralukast, pobilukast and SKB-106,203), leukotriene biosynthesis inhibitors (zileuton, BAY-1005); (f) non-steroidal anti-inflammatory agents (NSAIDs) such as propionic acid derivatives (e.g., alminoprofen, benoxaprofen, bucloxic acid, carprofen, fenbufen, fenoprofen, fluprofen, flurbiprofen, ibuprofen, indoprofen, ketoprofen, rniroprofen, naproxen, oxaprozin, pirprofen, pranoprofen, suprofen, tiaprofenic acid and tioxaprofen), acetic acid derivatives (e.g., indomethacin, acemetacin, alclofenac, clidanac, diclofenac, fenclofenac, fenclozic acid, fentiazac, furofenac, ibufenac, isoxepac, oxpinac, sulindac, tiopinac, tolmetin, zidometacin and zomepirac), fenamic acid derivatives (e.g., flufenamic acid, meclofenamic acid, mefenamic acid, niflumic acid and tolfenamic acid), biphenylcarboxylic acid derivatives (e.g., diflunisal and flufenisal), oxicams (e.g., isoxicam, piroxicam, sudoxicam and tenoxican), salicylates (e.g., acetyl salicylic acid and sulfasalazine) and the pyrazolones (e.g., apazone, bezpiperylon, feprazone, mofebutazone, oxyphenbutazone and phenylbutazone); (g) cyclooxygenase-2 (COX-2) inhibitors such as celecoxib (Celebrex®) and rofecoxib (Vioxx®); (h) inhibitors of phosphodiesterase type IV (PDE IV); (i) gold compounds such as auranofin and aurothioglucose, (j) TNF-alpha modulators such as etanercept (Enbrel®), (k) antibody therapies such as orthoclone (OKT3), daclizumab (Zenapax®), basiliximab (Simulect®), B cell modulators such as rituximab (Rituxan®), and infliximab (Remicade®), (1) other antagonists of the chemokine receptors, especially CCR1, CCR5, CXCR2, CXCR3, CCR2, CCR3, CCR(4), CCR7, CCR9, CX₃CR1 and CXCR6; (m) lubricants or emollients such as petrolatum and lanolin, (n) keratolytic agents (e.g., tazarotene), (o) vitamin D3 derivatives, e.g., calcipotriene or calcipotriol (Dovonex®), (p) PUVA, (q) anthralin (Drithrocreme®), (r) etretinate (Tegison®) and isotretinoin and (s) multiple sclerosis therapeutic agents such as interferon β-1β (Betaseron®), interferon (β-1α (Avonex®), azathioprine (Imurek®, Imuran®), glatiramer acetate (Capoxone®), a glucocorticoid (e.g., prednisolone) and cyclophosphamide (t) DMARDS such as methotrexate (u) T cell costimulatory modulators such as abatacept (Orencia®), (v) other compounds such as 5-aminosalicylic acid and prodrugs thereof; hydroxychloroquine; D-penicillamine; antimetabolites such as azathioprine, 6-mercaptopurine and methotrexate; DNA synthesis inhibitors such as hydroxyurea and microtubule disrupters such as colchicine. The weight ratio of the compound of the present disclosure to the second active ingredient may be varied and will depend upon the effective dose of each ingredient. Generally, an effective dose of each will be used. Thus, for example, when a compound of the present disclosure is combined with an NSAID the weight ratio of the compound of the present disclosure to the NSAID will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used.

### III. EXAMPLES

The following examples are offered to illustrate, but not to limit the claimed disclosure.

Reagents and solvents used below can be obtained from commercial sources such as Aldrich Chemical Co. (Milwaukee, Wisconsin, USA). ¹H-NMR spectra were recorded on a Varian Mercury 400 MHz NMR spectrometer. Significant peaks are provided relative to TMS and are tabulated in the order: multiplicity (s, singlet; d, doublet; t, triplet; q, quartet; m, multiplet) and number of protons. Mass spectrometry results are reported as the ratio of mass over charge, followed by the relative abundance of each ion (in parenthesis). In the examples, a single m/e value is reported for the M+H (or, as noted, M−H) ion containing the most common atomic isotopes. Isotope patterns correspond to the expected formula in all cases. Electrospray ionization (ESI) mass spectrometry analysis was conducted on a Hewlett-Packard MSD electrospray mass spectrometer using the HP1100 HPLC for sample delivery. Normally the analyte was dissolved in methanol at 0.1 mg/mL and 1 microlitre was infused with the delivery solvent into the mass spectrometer, which scanned from 100 to 1500 daltons. All compounds could be analyzed in the positive ESI mode, using acetonitrile/water with 1% formic acid as the delivery solvent. The compounds provided below could also be analyzed in the negative ESI mode, using 2 mM NH₄OAc in acetonitrile/water as delivery system.

The following abbreviations are used in the Examples and throughout the description of the disclosure: rt, room temperature; HPLC, high pressure liquid chromatography; TFA, trifluoroacetic acid; LC-MSD, liquid chromatograph/mass selective detector; LC-MS, liquid chromatograph/mass spectrometer; Pd₂dba₃, tris(dibenzylideneacetone) dipalladium; THF, tetrahydrofuran; DMF, dimethylformamide or N,N-dimethylformamide; DCM, dichloromethane; DMSO, dimethyl sulfoxide; TLC, thin-layer chromatography; KHMDS, potassium hexamethyldisilazane; ES, electrospray; sat., saturated.

Compounds within the scope of this disclosure can be synthesized as described below, using a variety of reactions known to the skilled artisan. One skilled in the art will also recognize that alternative methods may be employed to synthesize the target compounds of this disclosure, and that the approaches described within the body of this document are not exhaustive, but do provide broadly applicable and practical routes to compounds of interest.

Certain molecules claimed in this patent can exist in different tautomeric, enantiomeric and diastereomeric forms and all such variants of these compounds are claimed.

The detailed description of the experimental procedures used to synthesize key compounds in this text lead to molecules that are described by the physical data identifying them as well as by the structural depictions associated with them.

Those skilled in the art will also recognize that during standard work up procedures in organic chemistry, acids and bases are frequently used. Salts of the parent compounds are sometimes produced, if they possess the necessary intrinsic acidity or basicity, during the experimental procedures described within this patent.

### SYNTHETIC EXAMPLES

### Example 1: Synthesis of tert-butyl (R)-2-(4-(5-chloro-4-(((R)-1-(2,4-dichlorophenyl)ethyl)amino)pyrimidin-2-yl)piperazine-1-carbonyl)pyrrolidine-1-carboxylate

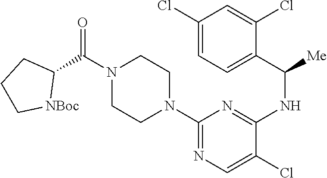 [SMILES: C[C@@H](Nc1nc(N2CCN(C(=O)[C@H]3CCCN3C(=O)OC(C)(C)C)CC2)ncc1Cl)c1ccc(Cl)cc1Cl]

Step a: To a solution of 2,4,5-trichloropyrimidine (5.0 g, 27.3 mmol) and (R)-1-(2,4-dichlorophenyl)ethan-1-amine (5.18 g, 27.3 mmol) in MeCN (100 mL) was added trimethylamine (3.8 mL, 27.6 mmol). The reaction mixture was stirred at room temperature for 16 h. The contents were concentrated and the solid was filtered off. The filtrate was purified by silica gel column chromatography to give (R)-2,5-dichloro-N-(1-(2,4-dichlorophenyl)ethyl)pyrimidin-4-amine. MS: (ES) m/z calculated for C₁₂H₁₀Cl₄N₃ [M+H]⁺ 336.0, found 336.0.

Step b: To a solution of (R)-2,5-dichloro-N-(1-(2,4-dichlorophenyl)ethyl)pyrimidin-4-amine (1.0 g, 2.97 mmol) and tert-butyl piperazine-1-carboxylate (0.55 g, 2.95 mmol) in DMSO (30 mL) was added N,N-diisopropylethylamine (2.6 mL, 14.9 mmol) and cesium fluoride (0.45 g, 2.96 mmol). The reaction mixture was heated at 100° C. for 3 h and was then diluted with water and ethyl acetate. The organic and aqueous layers were separated and the aqueous layer was extracted with ethyl acetate. The combined organic layers were dried with sodium sulfate, filtered and concentrated. The crude material was purified by silica gel column chromatography to provide tert-butyl (R)-4-(5-chloro-4-((1-(2,4-dichlorophenyl)ethyl)amino)pyrimidin-2-yl)piperazine-1-carboxylate. MS: (ES) m/z calculated for C₂₁H₂₇Cl₃N₅O₂ [M+H]⁺ 486.1, found 486.2.

Step c: To a solution of tert-butyl (R)-4-(5-chloro-4-((1-(2,4-dichlorophenyl)ethyl)amino)pyrimidin-2-yl)piperazine-1-carboxylate (1.3 g, 2.67 mmol) in dioxane (3 mL) was added a solution of 4.0 M HCl in dioxane (5 mL, 19.3 mmol). The reaction mixture was stirred at room temperature for 16 h. The contents were concentrated to give (R)-5-chloro-N-(1-(2,4-dichlorophenyl)ethyl)-2-(piperazin-1-yl)pyrimidin-4-amine. MS: (ES) m/z calculated for C₁₆H₁₉Cl₃N₅ [M+H]⁺ 386.1, found 386.0.

Step d: To a solution of (R)-5-chloro-N-(1-(2,4-dichlorophenyl)ethyl)-2-(piperazin-1-yl)pyrimidin-4-amine (300 mg, 0.709 mmol) and (tert-butoxycarbonyl)-D-proline (168 mg, 0.78 mmol) in DCM (3 mL) was added 1-Ethyl-3-(3-dimethylaminopropyl)carbodiimide hydrochloride (190 mg, 0.99 mmol) and dimethylaminopyridine (90 mg, 0.74 mmol). The contents were stirred at room temperature for 16 h. The reaction mixture was concentrated and then diluted with ethyl acetate and water. The aqueous layer was extracted with ethyl acetate. The combined organic layers were dried with sodium sulfate, filtered and concentrated. The crude material was purified on silica gel column chromatography followed by preparative HPLC to produce tert-butyl (R)-2-(4-(5-chloro-4-(((R)-1-(2,4-dichlorophenyl)ethyl)amino)pyrimidin-2-yl)piperazine-1-carbonyl)pyrrolidine-1-carboxylate. ¹H NMR (400 MHz, CDCl₃) (mixture of rotamers) δ 7.85 (s, 0.5H), 7.84 (s, 0.5H), 7.38 (s, 0.5H), 7.37 (s, 0.5H), 7.23 (d, J=8.5 Hz, 1H), 7.19 (d, J=8.4 Hz, 1H), 5.53-5.39 (m, 2H), 4.67 (d, J=3.6 Hz, 0.5H), 4.55 (dd, J=9.3, 3.6 Hz, 0.5H), 3.75-3.36 (m, 9H), 2.25-2.07 (m, 1H), 2.07-1.94 (m, 1H), 1.92-1.78 (m, 2H), 1.58 (s, 1.5H), 1.53 (m, 2.5H), 1.46 (s, 4.5H), 1.38 (m, 4.5H). MS: (ES) m/z calculated for C₂₆H₃₄Cl₃N₆O₃ [M+H]⁺ 583.2, found 583.2.

### Example 2: Synthesis of 2-(4-(D-prolyl)piperazin-1-yl)-5-chloro-4-(((R)-1-(2,4-dichlorophenyl)ethyl)amino)pyrimidine

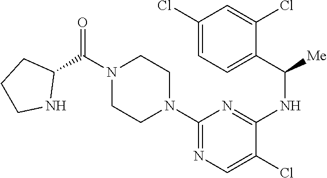 [SMILES: C[C@@H](Nc1nc(N2CCN(C(=O)[C@H]3CCCN3)CC2)ncc1Cl)c1ccc(Cl)cc1Cl]

To a solution of tert-butyl (R)-2-(4-(5-chloro-4-(((R)-1-(2,4-dichlorophenyl)ethyl)amino)pyrimidin-2-yl)piperazine-1-carbonyl)pyrrolidine-1-carboxylate (190 mg, 0.325 mmol) in dioxane (1 mL) was added 4.0 M HCl in dioxane (1.0 mL, 4.00 mmol). The reaction mixture was stirred at room temperature for 3 h and was then concentrated to dryness. The crude material was purified by preparative HPLC to yield 2-(4-(D-prolyl)piperazin-1-yl)-5-chloro-4-(((R)-1-(2,4-dichlorophenyl)ethyl)amino)pyrimidine. ¹H NMR (400 MHz, CDCl₃) δ 12.05 (s, 1H), 8.35 (s, 1H), 8.02 (d, J=1.9 Hz, 1H), 7.43 (d, J=2.0 Hz, 1H), 7.26-7.20 (m, 2H), 6.25 (d, J=6.4 Hz, 1H), 5.61-5.39 (m, 1H), 4.88 (bs, 1H), 3.96-3.81 (m, 3H), 3.75-3.27 (m, 6H), 2.59-2.43 (m, 1H), 2.19 (ddd, J=13.6, 7.2, 7.2 Hz, 1H), 2.12-1.99 (m, 1H), 1.92 (ddd, J=13.1, 6.6, 6.6 Hz, 1H), 1.61 (d, J=7.2 Hz, 3H). MS: (ES) m/z calculated for C₂₁H₂₆Cl₃N₆O [M+H]⁺ 483.1, found 483.1.

### Example 3: Synthesis of 5-chloro-4-(((R)-1-(2,4-dichlorophenyl)ethyl)amino)-2-(4-((2-hydroxyethyl)-D-prolyl)piperazin-1-yl)pyrimidine

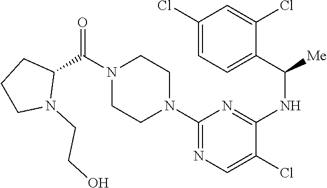 [SMILES: C[C@@H](Nc1nc(N2CCN(C(=O)[C@H]3CCCN3CCO)CC2)ncc1Cl)c1ccc(Cl)cc1Cl]

To a solution of 2-(4-(D-prolyl)piperazin-1-yl)-5-chloro-4-(((R)-1-(2,4-dichlorophenyl)ethyl)amino)pyrimidine (50 mg, 0.084 mmol) in MeCN (1 mL) was added potassium carbonate (35 mg, 0.25 mmol) and 2-iodoethanol (0.01 mL, 0.128 mmol). The reaction mixture was heated at 85° C. for 16 h and was then quenched with water. The contents were extracted with ethyl acetate and the combined organic layers were dried with sodium sulfate, filtered and concentrated. The crude material was purified by silica gel column chromatography followed by preparative HPLC to provide 5-chloro-4-(((R)-1-(2,4-dichlorophenyl)ethyl)amino)-2-(4-((2-hydroxyethyl)-D-prolyl)piperazin-1-yl)pyrimidine. ¹H NMR (400 MHz, CD₃OD) δ 7.96 (s, 1H), 7.49 (s, 1H), 7.41 (d, J=8.4 Hz, 1H), 7.32 (d, J=8.4 Hz, 1H), 5.62 (q, J=7.2 Hz, 1H), 4.84-4.75 (m, 2H), 4.02-3.73 (m, 6H), 3.65-3.57 (m, 3H), 3.56-3.40 (m, 2H), 3.37-3.34 (m, 1H), 3.28-3.23 (m, 1H), 2.69-2.50 (m, 1H), 2.28-2.13 (m, 1H), 2.09-1.90 (m, 2H), 1.59 (d, J=7.2 Hz, 3H). MS: (ES) m/z calculated for C₂₃H₃₀Cl₃N₆O₂ [M+H]⁺ 527.1, found 527.2.

### Example 4: Synthesis of 3-((R)-2-(4-(5-chloro-4-(((R)-1-(2,4-dichlorophenyl)ethyl)amino)pyrimidin-2-yl)piperazine-1-carbonyl)pyrrolidin-1-yl)propanoic acid

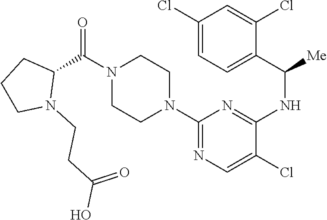 [SMILES: C[C@@H](Nc1nc(N2CCN(C(=O)[C@H]3CCCN3CCC(=O)O)CC2)ncc1Cl)c1ccc(Cl)cc1Cl]

To a solution of 2-(4-(D-prolyl)piperazin-1-yl)-5-chloro-4-(((R)-1-(2,4-dichlorophenyl)ethyl)amino)pyrimidine (50 mg, 0.084 mmol) in toluene (1 mL) was added N,N-diisopropylethylamine (0.03 mL, 0.167 mmol) and acrylic acid (0.01 mL, 0.146 mmol). The reaction mixture was heated at 85° C. for 16 h and was then concentrated to dryness. The crude material was purified by preparative HPLC to provide 3-((R)-2-(4-(5-chloro-4-(((R)-1-(2,4-dichlorophenyl)ethyl)amino)pyrimidin-2-yl)piperazine-1-carbonyl)pyrrolidin-1-yl)propanoic acid. ¹H NMR (400 MHz, CD₃OD) δ 7.95 (s, 1H), 7.48 (s, 1H), 7.41 (d, J=8.8 Hz, 1H), 7.31 (d, J=8.4, 1H), 5.61 (q, J=6.8 Hz, 1H), 4.82-4.68 (m, 1H), 3.96-3.75 (m, 5H), 3.75-3.62 (m, 1H), 3.62-3.53 (m, 2H), 3.53-3.39 (m, 3H), 3.28-3.19 (m, 1H), 2.82 (dd, J=6.6, 6.6 Hz, 2H), 2.68-2.55 (m, 1H), 2.26-2.16 (m, 1H), 2.06-1.93 (m, 2H), 1.58 (d, J=6.8 Hz, 3H). MS: (ES) m/z calculated for C₂₄H₃₀Cl₃N₆O₃ [M+H]⁺ 555.1, found 555.2.

### Example 5: Synthesis of tert-butyl (S)-2-(4-(5-chloro-4-(((R)-1-(2,4-dichlorophenyl)ethyl)amino)pyrimidin-2-yl)piperazine-1-carbonyl)pyrrolidine-1-carboxylate

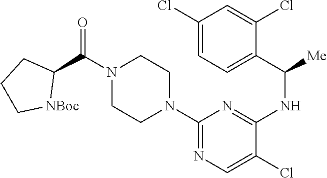 [SMILES: C[C@@H](Nc1nc(N2CCN(C(=O)[C@@H]3CCCN3C(=O)OC(C)(C)C)CC2)ncc1Cl)c1ccc(Cl)cc1Cl]

To a solution of (R)-5-chloro-N-(1-(2,4-dichlorophenyl)ethyl)-2-(piperazin-1-yl)pyrimidin-4-amine (300 mg, 0.709 mmol) and (tert-butoxycarbonyl)-L-proline (168 mg, 0.78 mmol) in DCM (3 mL) was added 1-ethyl-3-(3-dimethylaminopropyl)carbodiimide hydrochloride (190 mg, 0.99 mmol) and dimethylaminopyridine (90 mg, 0.74 mmol). The contents were stirred at room temperature for 16 h. The reaction mixture was concentrated and then diluted with ethyl acetate and water. The aqueous layer was extracted with ethyl acetate. The combined organic layers were dried with sodium sulfate, filtered and concentrated. The crude material was purified on silica gel column chromatography followed by preparative HPLC to produce tert-butyl (S)-2-(4-(5-chloro-4-(((R)-1-(2,4-dichlorophenyl)ethyl)amino)pyrimidin-2-yl)piperazine-1-carbonyl)pyrrolidine-1-carboxylate. ¹H NMR (400 MHz, CDCl₃) (mixture of rotamers) δ 7.85 (s, 0.5H), 7.84 (s, 0.5H), 7.39 (s, 0.5H), 7.37 (s, 0.5H), 7.24 (d, J=8.8 Hz, 1H), 7.19 (d, J=8.7 Hz, 1H), 5.54-5.37 (m, 2H), 4.67 (d, J=7.6 Hz, 0.5H), 4.54 (dd, J=8.8, 4.0 Hz, 0.5H), 3.75-3.40 (m, 9H), 3.32-3.22 (m, 1H), 2.25-2.08 (m, 1H), 2.07-1.94 (m, 1H), 1.92-1.76 (m, 2H), 1.57 (s, −2.5H), 1.54 (s, 1.5H), 1.46 (s, 4.5H), 1.39 (s, 4.5H). MS: (ES) m/z calculated for C₂₆H₃₄Cl₃N₆O₃ [M+H]⁺ 583.2, found 583.2.

### Example 6: Synthesis of 2-(4-(L-prolyl)piperazin-1-yl)-5-chloro-4-(((R)-1-(2,4-dichlorophenyl)ethyl)amino)pyrimidine

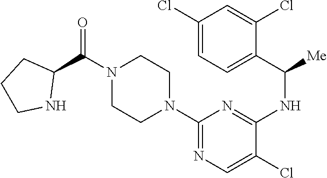 [SMILES: C[C@@H](Nc1nc(N2CCN(C(=O)[C@@H]3CCCN3)CC2)ncc1Cl)c1ccc(Cl)cc1Cl]

To a solution of tert-butyl (S)-2-(4-(5-chloro-4-(((R)-1-(2,4-dichlorophenyl)ethyl)-amino)pyrimidin-2-yl)piperazine-1-carbonyl)pyrrolidine-1-carboxylate (377 mg, 10.34 mmol) in dioxane (2 mL) was added 4.0 M HCl in dioxane (2.6 mL, 10.3 mmol). The reaction mixture was stirred at room temperature for 16 h and was then concentrated to dryness. The crude material was purified by preparative HPLC to yield 2-(4-(L-prolyl)piperazin-1-yl)-5-chloro-4-(((R)-1-(2,4-dichlorophenyl)ethyl)amino)pyrimidine. ¹H NMR (400 MHz, CDCl₃) δ 12.09 (s, 1H), 8.03 (s, 1H), 7.43 (s, 1H), 7.25-7.20 (m, 2H), 6.23 (m, 1H), 5.52-5.44 (m, 1H), 4.90 (s, 1H), 3.97-3.83 (m, 3H), 3.73-3.64 (m, 1H), 3.63-3.40 (m, 5H), 3.37-3.27 (m, 1H), 2.58-2.46 (m, 1H), 2.20 (ddd, J=14.0, 7.1, 7.1 Hz, 1H), 2.07 (ddd, J=7.4, 6.7, 6.7 Hz, 1H), 1.95-1.85 (m, 1H), 1.62 (d, J=6.9, 3H). MS: (ES) m/z calculated for C₂₁H₂₆Cl₃N₆O [M+H]⁺ 483.1, found 483.1.

### Example 7: Synthesis of 1-(4-(5-chloro-4-(((R)-1-(2,4-dichlorophenyl)ethyl)amino)pyrimidin-2-yl)piperazin-1-yl)-2-((R)-pyrrolidin-2-yl)ethan-1-one

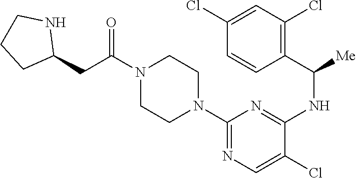 [SMILES: C[C@@H](Nc1nc(N2CCN(C(=O)C[C@H]3CCCN3)CC2)ncc1Cl)c1ccc(Cl)cc1Cl]

Step a: To a mixture of (R)-5-chloro-N-(1-(2,4-dichlorophenyl)ethyl)-2-(piperazin-1-yl)pyrimidin-4-amine hydrogen chloride (40 mg, 0.090 mmol), (R)-2-(1-(tert-butoxycarbonyl)pyrrolidin-2-yl)acetic acid (30 mg, 0.13 mmol) and HATU (80 mg, 0.21 mmol) in DMF (2 mL) was added triethylamine (0.080 mL, 0.57 mmol). The mixture was stirred at room temperature for 1 h, quenched with sat. NaHCO₃ and extracted with EtOAc. The organic layer was separated and filtered over MgSO₄. The filtrate was collected, concentrated under reduced pressure and purified by silica gel column to give tert-butyl (R)-2-(2-(4-(5-chloro-4-(((R)-1-(2,4-dichlorophenyl)ethyl)amino)pyrimidin-2-yl)piperazin-1-yl)-2-oxoethyl)pyrrolidine-1-carboxylate. MS: (ES) m/z calculated for C₂₇H₃₆Cl₃N₆O₃ [M+H]⁺ 597.2, found 597.3.

Step b: tert-Butyl (R)-2-(2-(4-(5-chloro-4-(((R)-1-(2,4-dichlorophenyl)ethyl)amino)pyrimidin-2-yl)piperazin-1-yl)-2-oxoethyl)pyrrolidine-1-carboxylate (50 mg, 0.080 mmol) was added to a 4.0 M solution of HCl in dioxane (2 mL, 8 mmol). The resulting solution was stirred at room temperature for 0.5 h, evaporated to dryness and purified by preparative HPLC to give 1-(4-(5-chloro-4-(((R)-1-(2,4-dichlorophenyl)ethyl)amino)pyrimidin-2-yl)piperazin-1-yl)-2-((R)-pyrrolidin-2-yl)ethan-1-one.

¹H NMR (400 MHz, CD₃OD) δ 7.99 (s, 1H), 7.49 (s, 1H), 7.41 (d, J=8.4 Hz, 1H), 7.33 (d, J=8.4 Hz, 1H), 5.63 (q, J=7.2 Hz, 1H), 3.85-3.95 (m, 1H), 3.50-3.82 (m, 9H), 3.22-3.33 (m, 1H), 3.00-3.08 (m, 1H), 2.80-2.87 (m, 1H), 2.20-2.30 (m, 1H), 2.03-2.15 (m, 1H), 1.92-2.02 (m, 1H), 1.71-1.82 (m, 1H), 1.60 (d, J=7.2 Hz, 3H). MS: (ES) m/z calculated for C₂₂H₂₈Cl₃N₆O [M+H]⁺ 497.1, found 497.2.

### Example 8: Synthesis of ((R)-azetidin-2-yl)(4-(5-chloro-4-(((R)-1-(2,4-dichlorophenyl)ethyl)amino)pyrimidin-2-yl)piperazin-1-yl)methanone

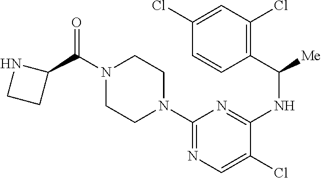 [SMILES: C[C@@H](Nc1nc(N2CCN(C(=O)[C@H]3CCN3)CC2)ncc1Cl)c1ccc(Cl)cc1Cl]

Step a: To a mixture of (R)-5-chloro-N-(1-(2,4-dichlorophenyl)ethyl)-2-(piperazin-1-yl)pyrimidin-4-amine hydrogen chloride (40 mg, 0.090 mmol), (R)-1-(tert-butoxycarbonyl)azetidine-2-carboxylic acid (30 mg, 0.15 mmol) and HATU (80 mg, 0.21 mmol) in DMF (2 mL) was added triethylamine (0.080 mL, 0.57 mmol). The mixture was stirred at room temperature for 1 h, quenched with sat. NaHCO₃ and extracted with EtOAc. The organic layer was separated and filtered over MgSO₄. The filtrate was collected, concentrated under reduced pressure and purified by silica gel column chromatography to give tert-butyl (R)-2-(4-(5-chloro-4-(((R)-1-(2,4-dichlorophenyl)ethyl)amino)pyrimidin-2-yl)piperazine-1-carbonyl)azetidine-1-carboxylate. MS: (ES) m/z calculated for C₂₅H₃₂Cl₃N₆O₃ [M+H]⁺ 569.2, found 569.2.

Step b: tert-butyl (R)-2-(4-(5-chloro-4-(((R)-1-(2,4-dichlorophenyl)ethyl)amino)pyrimidin-2-yl)piperazine-1-carbonyl)azetidine-1-carboxylate (40 mg, 0.070 mmol) was added to a solution of 4.0 M HCl in dioxane (2 mL, 8 mmol). The resulting solution was stirred at room temperature for 0.5 h, evaporated to dryness and purified by reverse phase HPLC to give ((R)-azetidin-2-yl)(4-(5-chloro-4-(((R)-1-(2,4-dichlorophenyl)ethyl)amino)pyrimidin-2-yl)piperazin-1-yl)methanone. ¹H NMR (400 MHz, CD₃OD) δ 7.99 (s, 1H), 7.50 (s, 1H), 7.42 (d, J=8.4 Hz, 1H), 7.32 (d, J=8.4 Hz, 1H), 5.59-5.66 (m, 1H), 5.51-5.50 (m, 1H), 4.10-4.19 (m, 1H), 3.88-3.98 (m, 1H), 3.74-3.84 (m, 3H), 3.61-3.70 (m, 2H), 3.46-3.56 (m, 1H), 3.34-3.45 (m, 2H), 2.86-2.97 (m, 1H), 2.52-2.67 (m, 1H), 1.60 (d, J=7.2 Hz, 3H). MS: (ES) m/z calculated for C₂₀H₂₄Cl₃N₆O [M+H]⁺ 469.1, found 469.1.

### Example 9: Synthesis of (R)-(4-(5-chloro-4-((1-(2,4-dichlorophenyl)ethyl)amino)pyrimidin-2-yl)piperazin-1-yl)(3-methylazetidin-3-yl)methanone

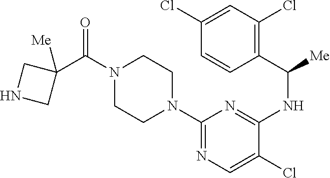 [SMILES: C[C@@H](Nc1nc(N2CCN(C(=O)C3(C)CNC3)CC2)ncc1Cl)c1ccc(Cl)cc1Cl]

Step a: To a mixture of (R)-5-chloro-N-(1-(2,4-dichlorophenyl)ethyl)-2-(piperazin-1-yl)pyrimidin-4-amine hydrogen chloride (50 mg, 0.12 mmol), 1-(tert-butoxycarbonyl)azetidine-3-carboxylic acid (38 mg, 0.19 mmol) and HATU (100 mg, 0.26 mmol) in DMF (2 mL) was added triethylamine (0.10 mL, 0.72 mmol). The mixture was stirred at room temperature for 1 h, quenched with sat. NaHCO₃ and extracted with EtOAc. The organic layer was separated and filtered over MgSO₄. The filtrate was collected, concentrated under reduced pressure and purified by silica gel column chromatography to give tert-butyl (R)-3-(4-(5-chloro-4-((1-(2,4-dichlorophenyl)ethyl)amino)pyrimidin-2-yl)piperazine-1-carbonyl)-3-methylazetidine-1-carboxylate. MS: (ES) m/z calculated for C₂₆H₃₄Cl₃N₆O₃ [M+H]⁺ 583.2, found 583.3.

Step b: tert-butyl (R)-3-(4-(5-chloro-4-((1-(2,4-dichlorophenyl)ethyl)amino)pyrimidin-2-yl)piperazine-1-carbonyl)-3-methylazetidine-1-carboxylate (65 mg, 0.11 mmol) was added to a solution of 4.0M HCl in dioxane (2 mL, 8 mmol). The resulting solution was stirred at room temperature for 0.5 h, evaporated to dryness and purified by preparative HPLC to give (R)-(4-(5-chloro-4-((1-(2,4-dichlorophenyl)ethyl)amino)pyrimidin-2-yl)piperazin-1-yl)(3-methylazetidin-3-yl)methanone. ¹H NMR (400 MHz, CD₃OD) δ 7.97 (s, 1H), 7.50 (s, 1H), 7.41 (d, J=8.4 Hz, 1H), 7.31 (d, J=8.4 Hz, 1H), 5.56-5.64 (m, 1H), 5.49 (s, 1H), 4.48 (d, J=10.8 Hz, 2H), 3.92 (d, J=10.8 Hz, 2H), 3.45-3.70 (m, 5H), 3.16-3.40 (m, 2H), 1.66 (s, 3H), 1.59 (d, J=7.2 Hz, 3H). MS: (ES) m/z calculated for C₂₁H₂₆Cl₃N₆O [M+H]⁺ 483.1, found 483.1.

### Example 10: Synthesis of (R)-2-amino-1-(4-(5-chloro-4-((1-(2,4-dichlorophenyl)ethyl)amino)pyrimidin-2-yl)piperazin-1-yl)-2-methylpropan-1-one

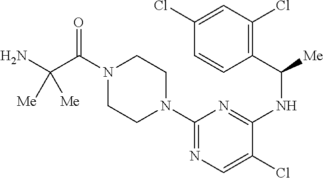 [SMILES: C[C@@H](Nc1nc(N2CCN(C(=O)C(C)(C)N)CC2)ncc1Cl)c1ccc(Cl)cc1Cl]

Step a: To a mixture of (R)-5-chloro-N-(1-(2,4-dichlorophenyl)ethyl)-2-(piperazin-1-yl)pyrimidin-4-amine hydrogen chloride (40 mg, 0.090 mmol), 2-((tert-butoxycarbonyl)amino)-2-methylpropanoic acid (39 mg, 0.19 mmol) and HATU (54 mg, 0.14 mmol) in DMF (2 mL) was added triethylamine (0.040 mL, 0.29 mmol). The mixture was stirred at room temperature for 1 h, quenched with sat. NaHCO₃ and extracted with EtOAc. The organic layer was separated and filtered over MgSO₄. The filtrate was collected, concentrated under reduced pressure and purified by silica gel column chromatography to give tert-butyl (R)-(1-(4-(5-chloro-4-((1-(2,4-dichlorophenyl)ethyl)amino)pyrimidin-2-yl)piperazin-1-yl)-2-methyl-1-oxopropan-2-yl)carbamate. MS: (ES) m/z calculated for C₂₅H₃₄Cl₃N₆O₃ [M+H]⁺ 571.2, found 571.2.

Step b: tert-butyl (R)-(1-(4-(5-chloro-4-((1-(2,4-dichlorophenyl)ethyl)amino)pyrimidin-2-yl)piperazin-1-yl)-2-methyl-1-oxopropan-2-yl)carbamate (37 mg, 0.064 mmol) was added to a 4.0M solution of HCl in dioxane (2 mL, 8 mmol). The mixture was stirred at room temperature for 0.5 h, evaporated to dryness under high vacuum and purified by preparative HPLC to give (R)-2-amino-1-(4-(5-chloro-4-((1-(2,4-dichlorophenyl)ethyl)amino)pyrimidin-2-yl)piperazin-1-yl)-2-methylpropan-1-one. ¹H NMR (400 MHz, CD₃OD) δ 7.99 (s, 1H), 7.50 (s, 1H), 7.42 (d, J=8.4 Hz, 1H), 7.30-7.35 (m, 1H), 5.63 (q, J=7.2 Hz, 1H), 3.62-3.78 (m, 8H), 1.68 (s, 6H), 1.60 (d, J=7.2 Hz, 3H). MS: (ES) m/z calculated for C₂₀H₂₆Cl₃N₆O [M+H]⁺ 471.1, found 471.1.

### Example 11: Synthesis of (R)-1-(4-(5-chloro-4-((1-(2,4-dichlorophenyl)ethyl)amino)pyrimidin-2-yl)piperazin-1-yl)-2-(dimethylamino)ethan-1-one

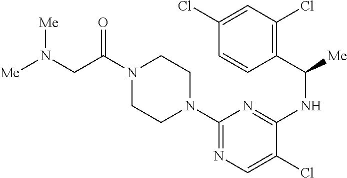 [SMILES: C[C@@H](Nc1nc(N2CCN(C(=O)CN(C)C)CC2)ncc1Cl)c1ccc(Cl)cc1Cl]

To a mixture of (R)-5-chloro-N-(1-(2,4-dichlorophenyl)ethyl)-2-(piperazin-1-yl)pyrimidin-4-amine hydrogen chloride (35 mg, 0.080 mmol), dimethylglycine (15 mg, 0.15 mmol) and HATU (50 mg, 0.13 mmol) in DMF (2 mL) was added triethylamine (0.050 mL, 0.36 mmol). The mixture was stirred at room temperature for 1 h, quenched with sat. NaHCO₃ and extracted with EtOAc. The organic layer was separated and filtered over MgSO₄. The filtrate was collected, concentrated under reduced pressure and purified by silica gel column chromatography to give (R)-1-(4-(5-chloro-4-((1-(2,4-dichlorophenyl)ethyl)amino)pyrimidin-2-yl)piperazin-1-yl)-2-(dimethylamino)ethan-1-one. ¹H NMR (400 MHz, CDCl₃) δ 7.84 (s, 1H), 7.38 (s, 1H), 7.17-7.27 (m, 2H), 5.38-5.51 (m, 2H), 3.37-3.68 (m, 8H), 3.12 (s, 2H), 2.29 (s, 6H), 1.53 (d, J=6.4 Hz, 3H). MS: (ES) m/z calculated for C₂₀H₂₆Cl₃N₆O [M+H]⁺ 471.1, found 471.1.

### Example 12: Synthesis of (4-(5-chloro-4-(((R)-1-(2,4-dichlorophenyl)ethyl)amino)pyrimidin-2-yl)piperazin-1-yl)((R)-piperidin-2-yl)methanone

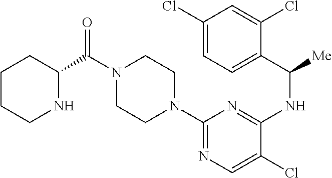 [SMILES: C[C@@H](Nc1nc(N2CCN(C(=O)[C@H]3CCCCN3)CC2)ncc1Cl)c1ccc(Cl)cc1Cl]

To a solution of (R)-5-chloro-N-(1-(2,4-dichlorophenyl)ethyl)-2-(piperazin-1-yl)pyrimidin-4-amine (200 mg, 0.47 mmol) and (R)-1-(tert-butoxycarbonyl)piperidine-2-carboxylic acid (112 mg, 0.49 mmol) in DCM (2 mL) was added 1-ethyl-3-(3-dimethylaminopropyl)carbodiimide hydrochloride (127 mg, 0.82 mmol) and dimethylaminopyridine (58 mg, 0.48 mmol). The reaction mixture was stirred at room temperature for 3 h, then concentrated to dryness. The material was diluted with ethyl acetate and water and the aqueous layer was extracted with ethyl acetate. The combined organic layers were dried with sodium sulfate, filtered and concentrated. The crude material was purified by preparative HPLC to give (4-(5-chloro-4-(((R)-1-(2,4-dichlorophenyl)ethyl)amino)pyrimidin-2-yl)piperazin-1-yl)((R)-piperidin-2-yl)methanone. ¹H NMR (400 MHz, CD₃OD) δ 7.93 (s, 1H), 7.48 (s, 1H), 7.40 (d, J=8.4 Hz, 1H), 7.31 (d, J=8.4, 1H), 5.60 (q, J=7.2 Hz, 1H), 4.34 (d, J=11.2 Hz, 1H), 3.92-3.81 (m, 1H), 3.81-3.70 (m, 2H), 3.64-3.48 (m, 5H), 3.46-3.34 (m, 2H), 3.05 (dd, J=13.6, 10.0 Hz, 1H), 2.12 (d, J=14.4 Hz, 1H), 2.00-1.84 (m, 2H), 1.80-1.68 (m, 2H), 1.67-1.61 (m, 1H), 1.58 (d, J=7.2 Hz, 3H). MS: (ES) m/z calculated for C₂₂H₂₈Cl₃N₆O [M+H]⁺ 497.1, found 497.2.

### Example 13: Synthesis of 2-(4-(D-prolyl)piperazin-1-yl)-5-chloro-4-(((R)-1-(2,4-dichlorophenyl)ethyl)amino)-6-methylpyrimidine

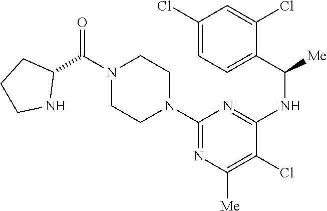 [SMILES: Cc1nc(N2CCN(C(=O)[C@H]3CCCN3)CC2)nc(N[C@H](C)c2ccc(Cl)cc2Cl)c1Cl]

Step a: To the solution of 2,4,5-trichloro-6-methyl-pyrimidine (3.00 g, 15.2 mmol) and triethylamine (3.2 mL, 22.8 mmol) in MeCN (60 mL) was added (1R)-1-(2,4-dichlorophenyl)ethanamine (2.89 g, 15.2 mmol). The reaction mixture was stirred at room temperature overnight. The reaction mixture was evaporated and purified by silica gel column chromatography to yield 2,5-dichloro-N-[(1R)-1-(2,4-dichlorophenyl)ethyl]-6-methyl-pyrimidin-4-amine). MS: (ES) m/z calculated for C₁₃H₁₂Cl₄N₃ [M+H]⁺ 350.0, found 350.0.

Step b: A mixture of 2,5-dichloro-N-[(1R)-1-(2,4-dichlorophenyl)ethyl]-6-methyl-pyrimidin-4-amine (400 mg, 1.14 mmol), tert-butyl piperazine-1-carboxylate (212 mg, 1.14 mmol), CsF (173 mg, 1.14 mmol) and N,N-diisopropylethylamine (0.61 mL, 3.42 mmol) in DMSO (3 mL) was heated at 100° C., overnight. The contents were diluted with ethyl acetate and water. The aqueous layer was extracted with ethyl acetate and the combined organic layers were dried with sodium sulfate, filtered and concentrated. The crude material was purified by silica gel column chromatography to yield tert-butyl 4-[5-chloro-4-[[(1R)-1-(2,4-dichlorophenyl)ethyl]amino]-6-methyl-pyrimidin-2-yl]piperazine-1-carboxylate. MS: (ES) m/z calculated for C₂₂H₂₉Cl₃N₅O₂ [M+H]⁺ 500.1, found 500.1.

Step c: To a solution of tert-butyl 4-[5-chloro-4-[[(1R)-1-(2,4-dichlorophenyl)ethyl]amino]-6-methyl-pyrimidin-2-yl]piperazine-1-carboxylate (560 mg, 1.12 mmol) in DCM (0.5 mL) was added 4.0 M HCl in dioxane (3.0 mL, 12 mmol). The mixture was stirred at room temperature for 2 h and then concentrated down to dryness to provide 5-chloro-N-[(1R)-1-(2,4-dichlorophenyl)ethyl]-6-methyl-2-piperazin-1-yl-pyrimidin-4-amine hydrochloride (480 mg, 1.10 mmol). MS: (ES) m/z calculated for C₁₇H₂₁Cl₃N₅ [M+H]⁺ 400.1, found 400.0.

Step d: To a solution of 5-chloro-N-[(1R)-1-(2,4-dichlorophenyl)ethyl]-6-methyl-2-piperazin-1-yl-pyrimidin-4-amine (50 mg, 0.125 mmol) and (2R)-1-tert-butoxycarbonylpyrrolidine-2-carboxylic acid (27 mg, 0.125 mmol) in DMF (1 mL) was added DIPEA (0.065 mL, 0.374 mmol) and HATU (47 mg, 0.125 mmol). The reaction was stirred at room temperature for 1 h. The mixture was diluted with ethyl acetate and then washed with H₂O and brine. The organic layer dried over Na₂SO₄, filtered, and concentrated. The crude residue was purified by silica gel column chromatography to provide tert-butyl (2R)-2-[4-[5-chloro-4-[[(1R)-1-(2,4-dichlorophenyl)ethyl]amino]-6-methyl-pyrimidin-2-yl]piperazine-1-carbonyl]pyrrolidine-1-carboxylate. MS: (ES) m/z calculated for C₂₇H₃₆Cl₃N₆O₃ [M+H]⁺ 597.2, found 597.1.

Step e: A solution of tert-butyl (2R)-2-[4-[5-chloro-4-[[(1R)-1-(2,4-dichlorophenyl)ethyl]amino]-6-methyl-pyrimidin-2-yl]piperazine-1-carbonyl]pyrrolidine-1-carboxylate (70 mg, 0.117 mmol) and 4.0 M solution of HCl in dioxane (1.0 mL, 4.00 mmol) was stirred at room temperature for 2 h. The mixture was concentrated to dryness and the residue was purified by preparative HPLC to give 2-(4-(D-prolyl)piperazin-1-yl)-5-chloro-4-(((R)-1-(2,4-dichlorophenyl)ethyl)amino)-6-methylpyrimidine). ¹H NMR (400 MHz, CD₃OD) δ 7.48 (s, 1H), 7.40 (d, J=8.4 Hz, 1H), 7.31 (d, J=8.4 Hz, 1H), 5.59 (q, J=8.4 Hz, 1H), 4.71 (t, J=8.4 Hz, 1H), 3.92-3.30 (m, 12H), 2.57-2.45 (m, 1H), 2.43 (s, 3H), 2.16-1.88 (m, 3H), 1.58 (d, J=8.4 Hz, 3H). MS: (ES) m/z calculated for C₂₂H₂₈Cl₃N₆O [M+H]⁺ 497.1, found 497.1.

### Example 14: Synthesis of 2-((S)-4-(D-prolyl)-3-(hydroxymethyl)piperazin-1-yl)-5-chloro-4-(((R)-1-(2,4-dichlorophenyl)ethyl)amino)-6-methylpyrimidine

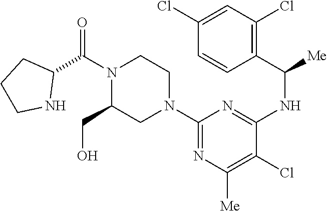 [SMILES: Cc1nc(N2CCN(C(=O)[C@H]3CCCN3)[C@H](CO)C2)nc(N[C@H](C)c2ccc(Cl)cc2Cl)c1Cl]

The title compound was prepared from the similar procedure by using tert-butyl (S)-2-(hydroxymethyl)piperazine-1-carboxylate as one of the starting material. ¹H NMR (400 MHz, CD₃OD) δ 7.47 (s, 1H), 7.39 (d, J=8.4 Hz, 1H), 7.30 (d, J=8.4 Hz, 1H), 5.56-5.52 (m, 1H), 4.80-4.36 (m, 4H), 4.25-4.00 (m, 1H), 3.81-3.55 (m, 3H), 3.52-3.30 (m, 4H), 3.22-2.80 (m, 2H), 2.57-2.45 (m, 5H), 2.43-2.38 (m, 3H), 2.16-1.92 (m, 3H), 1.57 (d, J=8.4 Hz, 3H). MS: (ES) m/z calculated for C₂₃H₂₉Cl₃N₆O₂ [M+H]⁺ 527.1, found 527.2.

### Example 15: Synthesis of (R)-N-(1-(5-chloro-4-(((R)-1-(2,4-dichlorophenyl)ethyl)amino)pyrimidin-2-yl)piperidin-4-yl)pyrrolidine-2-carboxamide

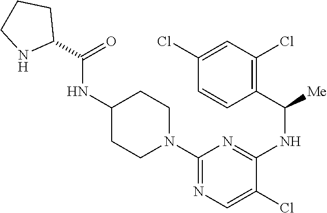

The title compound was prepared from the similar procedure by using tert-butyl piperidin-4-ylcarbamate and (R)-2,5-dichloro-N-(1-(2,4-dichlorophenyl)ethyl)pyrimidin-4-amine as starting materials. ¹H NMR (400 MHz, CD₃OD) δ 7.93 (s, 1H), 7.47 (s, 1H), 7.39 (d, J=8.4 Hz, 1H), 7.32 (d, J=8.4 Hz, 1H), 5.59 (q, J=8.4 Hz, 1H), 4.30-4.10 (m, 3H), 4.03-3.92 (m, 1H), 3.45-3.30 (m, 5H), 3.19 (t, J=13.0 Hz, 2H), 2.47-2.36 (m, 1H), 2.11-1.86 (m, 5H), 1.57 (d, J=8.4 Hz, 3H), 1.55-1.41 (m, 1H), 1.31-1.15 (m, 1H). MS: (ES) m/z calculated for C₂₂H₂₈Cl₃N₆O [M+H]⁺ 497.1, found 497.1.

### Example 16: Synthesis of 2-((R)-4-(D-prolyl)-2-methylpiperazin-1-yl)-5-chloro-4-(((R)-1-(2,4-dichlorophenyl)ethyl)amino)pyrimidine

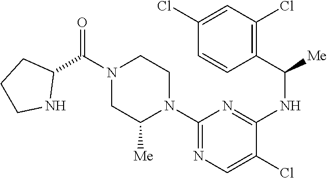 [SMILES: C[C@@H]1CN(C(=O)[C@H]2CCCN2)CCN1c1ncc(Cl)c(N[C@H](C)c2ccc(Cl)cc2Cl)n1]

The title compound was prepared from the similar procedure by using tert-butyl (R)-3-methylpiperazine-1-carboxylate and (R)-2,5-dichloro-N-(1-(2,4-dichlorophenyl)ethyl)pyrimidin-4-amine as starting materials. ¹H NMR (400 MHz, CD₃OD) δ 7.93 (s, 1H), 7.48 (d, J=4.0 Hz, 1H), 7.38 (d, J=8.4 Hz, 1H), 7.30 (d, J=8.4 Hz, 1H), 5.60-5.51 (m, 1H), 4.71-4.61 (m, 2H), 4.35-4.05 (m, 2H), 3.88-3.72 (m, 1H), 3.58-3.22 (m, 6H), 3.20-2.88 (m, 1H), 2.68-2.42 (m, 1H), 2.17-1.83 (m, 3H), 1.58 (d, J=7.1 Hz, 3H), 1.02-0.79 (m, 3H). MS: (ES) m/z calculated for C₂₂H₂₈Cl₃N₆O [M+H]⁺ 497.1, found 497.1.

### Example 17: Synthesis of 2-(D-prolyl)-5-(5-chloro-4-(((R)-1-(2,4-dichlorophenyl)ethyl)amino)pyrimidin-2-yl)-2,5-diazabicyclo[2.2.1]heptane

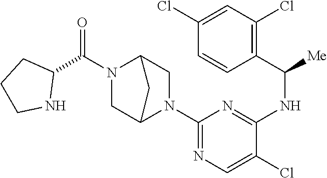 [SMILES: C[C@@H](Nc1nc(N2CC3CC2CN3C(=O)[C@H]2CCCN2)ncc1Cl)c1ccc(Cl)cc1Cl]

The title compound was prepared from the similar procedure by using tert-butyl 2,5-diazabicyclo[2.2.1]heptane-2-carboxylate and (R)-2,5-dichloro-N-(1-(2,4-dichlorophenyl)ethyl)pyrimidin-4-amine as starting materials. ¹H NMR (400 MHz, CD₃OD) δ 7.94 (s, 1H), 7.62-7.46 (m, 1H), 7.41-7.28 (m, 2H), 5.70-5.50 (m, 1H), 5.12-4.60 (m, 4H), 4.05-3.25 (m, 6H), 2.90-2.65 (m, 1H), 2.57-2.38 (m, 1H), 2.16-1.86 (m, 5H), 1.60 (s, 3H). MS: (ES) m/z calculated for C₂₂H₂₅Cl₃N₆O [M+H]⁺ 495.1, found 495.1.

### Example 18: Synthesis of [(2R)-4-[5-chloro-4-[[(1R)-1-(2,4-dichlorophenyl)ethyl]amino]pyrimidin-2-yl]-2-methyl-piperazin-1-yl]-[(2R)-pyrrolidin-2-yl]methanone

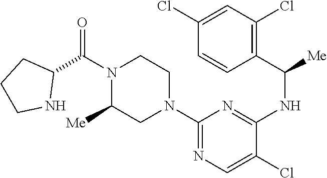 [SMILES: C[C@@H]1CN(c2ncc(Cl)c(N[C@H](C)c3ccc(Cl)cc3Cl)n2)CCN1C(=O)[C@H]1CCCN1]

Step a: To a mixture of 2,5-dichloro-N-[(1R)-1-(2,4-dichlorophenyl)ethyl]pyrimidin-4-amine (150 mg, 0.45 mmol) and N,N-diisopropylethylamine (0.093 mL, 0.53 mmol) in DMF (1 mL) was added tert-butyl (2R)-2-methylpiperazine-1-carboxylate (89 mg, 0.45 mmol). The mixture was stirred at 80° C. for 15 h, diluted with ethyl acetate (100 mL) and washed with H₂O and brine. The organic layer was dried over MgSO₄, filtered, and the filtrate was concentrated. The crude residue was purified by silica gel column chromatography to yield tert-butyl (2R)-4-[5-chloro-4-[[(1R)-1-(2,4-dichlorophenyl)ethyl]amino]pyrimidin-2-yl]-2-methyl-piperazine-1-carboxylate as a colorless oil. MS: (ES) m/z calculated for C₂₂H₂₉Cl₃N₅O₂ [M+H]⁺ 500.1, found 500.1.

Step b: A mixture of tert-butyl (2R)-4-[5-chloro-4-[[(1R)-1-(2,4-dichlorophenyl)ethyl]amino]pyrimidin-2-yl]-2-methyl-piperazine-1-carboxylate (170 mg, 0.34 mmol) in a solution of 4 N HCl in dioxane (2 mL, 8 mmol) was stirred at room temperature for 30 min. The mixture was diluted with diethyl ether and the solid product was filtered, washed with diethyl ether, and the residual solvent was removed in vacuo to yield 5-chloro-N-[(1R)-1-(2,4-dichlorophenyl)ethyl]-2-[(3R)-3-methylpiperazin-1-yl]pyrimidin-4-amine. MS: (ES) m/z calculated for C₁₇H₂₁Cl₃N₅ [M+H]⁺ 400.1, found 400.1.

Step c: To a mixture of 5-chloro-N-[(1R)-1-(2,4-dichlorophenyl)ethyl]-2-[(3R)-3-methylpiperazin-1-yl]pyrimidin-4-amine (62 mg, 0.14 mmol), (2R)-1-tert-butoxycarbonylpyrrolidine-2-carboxylic acid (31 mg, 0.14 mmol), and 1-ethyl-3-(3-dimethylaminopropyl)carbodiimide hydrochloride (38 mg, 0.20 mmol) in DCM (1 mL) was added 4-(dimethylamino)pyridine (18 mg, 0.15 mmol). The mixture was stirred at room temperature for 48 h. The reaction was quenched with saturated NH₄Cl(aq) and the aqueous mixture was extracted with DCM. The combined organic layers were dried over MgSO₄, filtered, and concentrated. The organic layer was dried over MgSO₄, filtered, and concentrated. The crude residue was purified by silica gel column chromatography to yield tert-butyl (2R)-2-[(2R)-4-[5-chloro-4-[[(1R)-1-(2,4-dichlorophenyl)ethyl]amino]pyrimidin-2-yl]-2-methyl-piperazine-1-carbonyl]pyrrolidine-1-carboxylate. MS: (ES) m/z calculated for C₂₇H₃₆Cl₃N₆O₃ [M+H]⁺ 597.2, found 597.2.

Step d: A mixture of tert-butyl (2R)-2-[(2R)-4-[5-chloro-4-[[(1R)-1-(2,4-dichlorophenyl)ethyl]amino]pyrimidin-2-yl]-2-methyl-piperazine-1-carbonyl]pyrrolidine-1-carboxylate (47 mg, 0.079 mmol) in a solution of 4 N HCl in dioxane (1 mL, 4 mmol) was stirred at room temperature for 30 min. The mixture was diluted with diethyl ether and the solid was filtered, washed with diethyl ether, and the residual solvent was removed in vacuo to yield [(2R)-4-[5-chloro-4-[[(1R)-1-(2,4-dichlorophenyl)ethyl]amino]pyrimidin-2-yl]-2-methyl-piperazin-1-yl]-[(2R)-pyrrolidin-2-yl]methanone. ¹H NMR (400 MHz, CD₃OD) δ 8.92 (s, 1H), 8.03 (s, 1H), 7.99 (s, 1H), 7.51 (s, 1H), 7.41 (d, J=7.9 Hz, 1H), 7.34 (d, J=9.4 Hz, 1H), 5.70-5.56 (m, 1H), 4.69-4.48 (m, 1H), 4.36-4.11 (m, 1H), 4.09-3.73 (m, 2H), 3.64-3.32 (m, 5H), 2.61-2.46 (m, 1H), 2.17-1.84 (m, 3H), 1.67-1.55 (m, 3H), 1.40-1.10 (m, 4H). MS: (ES) m/z calculated for C₂₂H₂₈Cl₃N₆O [M+H]⁺ 497.1, found 497.1.

### Example 19: Synthesis of [(2R)-4-[5-chloro-4-[[(1R)-1-(2,4-dichlorophenyl)ethyl]amino]pyrimidin-2-yl]-2-methyl-piperazin-1-yl]-piperazin-1-yl-methanone

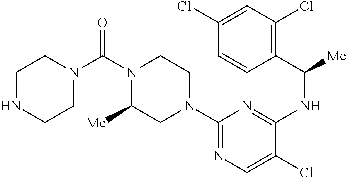 [SMILES: C[C@@H]1CN(c2ncc(Cl)c(N[C@H](C)c3ccc(Cl)cc3Cl)n2)CCN1C(=O)N1CCNCC1]

Step a: To a mixture of 5-chloro-N-[(1R)-1-(2,4-dichlorophenyl)ethyl]-2-[(3R)-3-methylpiperazin-1-yl]pyrimidin-4-amine dihydrochloride (80 mg, 0.17 mmol) and N,N-diisopropylethylamine (0.088 mL, 0.51 mmol) in DCM (1 mL) was added tert-butyl 4-chlorocarbonylpiperazine-1-carboxylate (75 mg, 0.30 mmol). The contents were stirred at room temperature for 9 h. The mixture was diluted with DCM and washed with H₂O and brine. The organic layer was concentrated and purified by silica gel column chromatography to yield tert-butyl 4-((R)-4-(5-chloro-4-(((R)-1-(2,4-dichlorophenyl)ethyl)amino)pyrimidin-2-yl)-2-methylpiperazine-1-carbonyl)piperazine-1-carboxylate. MS: (ES) m/z calculated for C₂₇H₃₇Cl₃N₇O₃ [M+H]⁺ 612.2, found 612.2.

Step b: A mixture of tert-butyl 4-((R)-4-(5-chloro-4-(((R)-1-(2,4-dichlorophenyl)ethyl)amino)pyrimidin-2-yl)-2-methylpiperazine-1-carbonyl)piperazine-1-carboxylate (30 mg, 0.049 mmol) in a solution of 4 N HCl in dioxane (1 mL, 4 mmol) was stirred at room temperature for 30 min. The mixture was diluted with diethyl ether and the solid product was filtered, washed with diethyl ether, and the residual solvent was removed in vacuo to yield [(2R)-4-[5-chloro-4-[[(1R)-1-(2,4-dichlorophenyl)ethyl]amino]pyrimidin-2-yl]-2-methyl-piperazin-1-yl]-piperazin-1-yl-methanone. ¹H NMR (400 MHz, CD₃OD) δ 8.06 (s, 1H), 7.95 (s, 1H), 7.51 (s, 1H), 7.42-7.38 (m, 1H), 7.36-7.31 (m, 1H), 5.60 (q, J=7.0 Hz, 1H), 4.08-3.92 (m, 3H), 3.56-3.34 (m, 7H), 3.28-3.18 (m, 5H), 3.13 (s, 1H), 1.60 (d, J=6.9 Hz, 3H), 1.18 (d, J=6.6 Hz, 3H). MS: (ES) m/z calculated for C₂₂H₂₉Cl₃N₇O [M+H]⁺ 512.1, found 512.1.

### Example 20: Synthesis of [(2R,5S)-4-[5-chloro-4-[[(1R)-1-(2,4-dichlorophenyl)ethyl]amino]pyrimidin-2-yl]-2,5-dimethyl-piperazin-1-yl]-[(2R)-pyrrolidin-2-yl]methanone

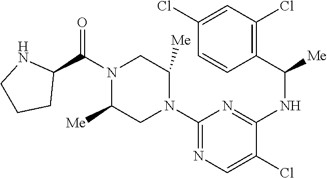 [SMILES: C[C@@H]1CN(c2ncc(Cl)c(N[C@H](C)c3ccc(Cl)cc3Cl)n2)[C@@H](C)CN1C(=O)[C@H]1CCCN1]

Step a: To a mixture of 2,5-dichloro-N-[(1R)-1-(2,4-dichlorophenyl)ethyl]pyrimidin-4-amine (300 mg, 0.89 mmol) and N,N-diisopropylethylamine (140 mg, 1.1 mmol) in DMF (5 mL) was added tert-butyl (2R,5S)-2,5-dimethylpiperazine-1-carboxylate (190 mg, 0.89 mmol). The contents were stirred at 100° C. for 18 h. The mixture was diluted with ethyl acetate and washed with H₂O and brine. The organic layer was dried over MgSO₄, filtered, and concentrated. The crude residue was purified by silica gel column chromatography to yield tert-butyl (2R,5S)-4-[5-chloro-4-[[(1R)-1-(2,4-dichlorophenyl)ethyl]amino]pyrimidin-2-yl]-2,5-dimethyl-piperazine-1-carboxylate. MS: (ES) m/z calculated for C₂₃H₃₁Cl₃N₅O₂ [M+H]⁺ 514.1, found 514.1.

Step b: A mixture of tert-butyl (2R,5S)-4-[5-chloro-4-[[(1R)-1-(2,4-dichlorophenyl)ethyl]amino]pyrimidin-2-yl]-2,5-dimethyl-piperazine-1-carboxylate (216 mg, 0.42 mmol) in a solution of 4 N HCl 1,4-dioxane (2 mL, 8 mmol) was stirred at room temperature for 1 h. The mixture was diluted with diethyl ether and the solid product was filtered, washed with diethyl ether, and the residual solvent was removed in vacuo to yield 5-chloro-N-((R)-1-(2,4-dichlorophenyl)ethyl)-2-((2S,5R)-2,5-dimethylpiperazin-1-yl)pyrimidin-4-amine. MS: (ES) m/z calculated for C₁₈H₂₃Cl₃N₅ [M+H]⁺ 414.1, found 414.1.

Step c: To a mixture of 5-chloro-N-((R)-1-(2,4-dichlorophenyl)ethyl)-2-((2S,5R)-2,5-dimethylpiperazin-1-yl)pyrimidin-4-amine (100 mg, 0.21 mmol), (2R)-1-tert-butoxycarbonylpyrrolidine-2-carboxylic acid (68 mg, 0.32 mmol), and 1-ethyl-3-(3-dimethylaminopropyl)carbodiimide hydrochloride (61 mg, 0.32 mmol) in DCM (1 mL) was added 4-(dimethylamino)pyridine (26 mg, 0.21 mmol). The mixture was stirred at room temperature for 48 h. The reaction was quenched with saturated NH₄Cl (aq) and the aqueous mixture was extracted with DCM. The combined organic layers were dried over MgSO₄, filtered, and concentrated. The crude residue was purified by silica gel column chromatography to yield tert-butyl (2R)-2-[(2R,5S)-4-[5-chloro-4-[[(1R)-1-(2,4-dichlorophenyl)ethyl]amino]pyrimidin-2-yl]-2,5-dimethyl-piperazine-1-carbonyl]pyrrolidine-1-carboxylate. MS: (ES) m/z calculated for C₂₈H₃₈Cl₃N₆O₃ [M+H]⁺ 611.2, found 611.2.

Step d: A mixture of tert-butyl (2R)-2-[(2R,5S)-4-[5-chloro-4-[[(1R)-1-(2,4-dichlorophenyl)ethyl]amino]pyrimidin-2-yl]-2,5-dimethyl-piperazine-1-carbonyl]pyrrolidine-1-carboxylate (64 mg, 0.11 mmol) in a solution of 4 N HCl in dioxane (1 mL, 4 mmol) was stirred at room temperature for 30 min. The mixture was diluted with diethyl ether and the solid product was filtered, washed with diethyl ether, and the residual solvent was removed in vacuo to yield [(2R,5S)-4-[5-chloro-4-[[(1R)-1-(2,4-dichlorophenyl)ethyl]amino]pyrimidin-2-yl]-2,5-dimethyl-piperazin-1-yl]-[(2R)-pyrrolidin-2-yl]methanone. ¹H NMR (400 MHz, DMSO-d₆) δ 8.78-8.41 (m, 1H), 7.95 (s, 1H), 7.59 (s, 1H), 7.53 (d, J=8.5, 1H), 7.43 (d, J=8.5 Hz, 1H), 5.55-5.36 (m, 1H), 4.86-3.95 (m, 7H), 3.30-3.11 (m, 3H), 2.47-2.19 (m, 2H), 2.04-1.83 (m, 3H), 1.85-1.64 (m, 1H), 1.48 (d, J=6.9 Hz, 3H), 1.28-0.92 (m, 4H). MS: (ES) m/z calculated for C₂₃H₃₀Cl₃N₆O [M+H]⁺ 511.2, found 511.1.

### Example 21: Synthesis of 2-(3-(4-(5-chloro-4-(((R)-1-(2,4-dichlorophenyl)ethyl)amino)pyrimidin-2-yl)-3,6-dihydropyridin-1(2H)-yl)piperidin-1-yl)ethan-1-ol

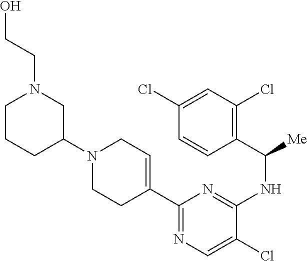 [SMILES: C[C@@H](Nc1nc(C2=CCN(C3CCCN(CCO)C3)CC2)ncc1Cl)c1ccc(Cl)cc1Cl]

Step a: To a mixture of pyridinium chlorochromate (30 g, 140 mmol) in DCM (300 mL) was added tert-butyl (3S)-3-(4-hydroxy-1-piperidyl)piperidine-1-carboxylate (10 g, 35 mmol). The contents were stirred at room temperature for 72 h and an aqueous solution of 2 M K₂CO₃ was added. The biphasic mixture was stirred for 2 h, the mixture was filtered through celite and the layers were separated. The aqueous layer was extracted with DCM and the organic layers were combined, dried over MgSO₄, filtered, and the filtrate was concentrated. The crude residue was purified by silica gel column chromatography to yield tert-butyl (3S)-3-(4-oxo-1-piperidyl)piperidine-1-carboxylate. MS: (ES) m/z calculated for C₁₅H₃₀N₂O₄ [M+H₃O]⁺301.2, found 301.2.

Step b: To a mixture of tert-butyl (3S)-3-(4-oxo-1-piperidyl)piperidine-1-carboxylate (6.3 g, 22 mmol) in THF (100 mL) at −78° C. under N₂ atmosphere was added solution of 1M lithium bis(trimethylsilyl)amide in THF (27 mL, 27 mmol), dropwise over 10 min. The mixture was stirred at −20° C. for 2 h, and then cooled to −78° C. A solution of N,N-bis(trifluoromethanesulfonyl)aniline (10 g, 29 mmol) in 40 mL THF was added dropwise and the mixture was stirred at room temperature for 6 h. The reaction was quenched with saturated NH₄Cl (aq) and the layers were separated. The organic layer was dried over Na₂SO₄, filtered, and the filtrate was concentrated. The crude residue was purified by silica gel column chromatography to yield tert-butyl (3S)-3-[4-(trifluoromethylsulfonyloxy)-3,6-dihydro-2H-pyridin-1-yl]piperidine-1-carboxylate. MS: (ES) m/z calculated for C₁₆H₂₆F₃N₂O₅S [M+H]⁺ 415.1, found 415.2.

Step c: A mixture of tert-butyl (3S)-3-[4-(trifluoromethylsulfonyloxy)-3,6-dihydro-2H-pyridin-1-yl]piperidine-1-carboxylate (1.0 g, 2.4 mmol), potassium phenoxide (480 mg, 3.6 mmol), triphenylphosphine (38 mg, 0.14 mmol), and bis(pinacolato)diboron (670 mg, 2.7 mmol) in toluene (6 mL) was sparged with N₂ gas 5 min. Pd(PPh₃)₂Cl₂ (51 mg, 0.072 mmol) was added, and the mixture was stirred at 50° C. for 3 h. The contents were diluted with H₂O and extracted with ethyl acetate. The organic layers were dried over MgSO₄, filtered, and the filtrate was concentrated. The crude residue was purified by silica gel column chromatography to yield tert-butyl (3S)-3-[4-(4,4,5,5-tetramethyl-1,3,2-dioxaborolan-2-yl)-3,6-dihydro-2H-pyridin-1-yl]piperidine-1-carboxylate. MS: (ES) m/z calculated for C₂₁H₃₈BN₂O₄ [M+H]⁺ 393.3, found 393.3.

Step d: A mixture of 2,5-dichloro-N-[(1R)-1-(2,4-dichlorophenyl)ethyl]pyrimidin-4-amine (260 mg, 0.76 mmol), tert-butyl (3S)-3-[4-(4,4,5,5-tetramethyl-1,3,2-dioxaborolan-2-yl)-3,6-dihydro-2H-pyridin-1-yl]piperidine-1-carboxylate (300 mg, 0.76 mmol), and potassium carbonate (320 mg, 2.3 mmol) in a 1:1 solution of 1,4-dioxane/H₂O (4 mL) was sparged with N₂ gas for 5 min. Pd(dppf)Cl₂-DCM complex (31 mg, 0.038 mmol) was added, and the mixture was stirred at 80° C. for 15 h. The contents were diluted with H₂O and extracted with ethyl acetate.

The organic layers were dried over MgSO₄, filtered, and the filtrate was concentrated. The crude residue was purified by silica gel column chromatography to yield tert-butyl 3-(4-(5-chloro-4-(((R)-1-(2,4-dichlorophenyl)ethyl)amino)pyrimidin-2-yl)-3,6-dihydropyridin-1(2H)-yl)piperidine-1-carboxylate. MS: (ES) m/z calculated for C₂₇H₃₅Cl₃N₅O₂ [M+H]⁺ 566.2, found 566.2.

Step e: A mixture of tert-butyl 3-(4-(5-chloro-4-(((R)-1-(2,4-dichlorophenyl)ethyl)amino)pyrimidin-2-yl)-3,6-dihydropyridin-1(2H)-yl)piperidine-1-carboxylate in a solution of 4 N HCl in dioxane (1 mL, 4 mmol) was stirred at room temperature for 30 min. The mixture was diluted with diethyl ether and the solid product was filtered, washed with diethyl ether, and the residual solvent was removed in vacuo to yield 5-chloro-N-((R)-1-(2,4-dichlorophenyl)ethyl)-2-(1-(piperidin-3-yl)-1,2,3,6-tetrahydropyridin-4-yl)pyrimidin-4-amine dihydrochloride. MS: (ES) m/z calculated for C₂₂H₂₇Cl₃N₅ [M+H]⁺ 466.1, found 466.1.

Step f: To a mixture of 5-chloro-N-((R)-1-(2,4-dichlorophenyl)ethyl)-2-(1-(piperidin-3-yl)-1,2,3,6-tetrahydropyridin-4-yl)pyrimidin-4-amine dihydrochloride (45 mg, 0.097 mmol), and potassium carbonate (41 mg, 0.3 mmol) in acetonitrile (0.5 mL) was added 2-iodoethanol (17 mg, 0.1 mmol). The mixture was stirred at 80° C. for 5 h. The crude mixture was purified by preparative HPLC to yield 2-(3-(4-(5-chloro-4-(((R)-1-(2,4-dichlorophenyl)ethyl)amino)pyrimidin-2-yl)-3,6-dihydropyridin-1(2H)-yl)piperidin-1-yl)ethan-1-ol. ¹H NMR (400 MHz, CD₃OD) δ 8.21 (s, 1H), 7.69-7.63 (m, 1H), 7.43 (s, 1H), 7.42-7.35 (m, 1H), 7.26 (d, J=8.6 Hz, 1H), 6.95 (s, 1H), 5.63 (q, J=7.0 Hz, 1H), 4.01-3.81 (m, 5H), 3.81-3.70 (m, 1H), 3.68-3.51 (m, 2H), 3.51-3.33 (m, 4H), 3.13 (s, 1H), 3.09-2.96 (m, 1H), 2.95-2.82 (m, 1H), 2.79-2.64 (m, 1H), 2.35-2.08 (m, 2H), 1.97-1.78 (m, 2H), 1.56 (d, J=7.2 Hz, 3H). MS: (ES) m/z calculated for C₂₄H₃₁Cl₃N₅O [M+H]⁺ 510.2, found 510.2.

### Example 22: Synthesis of 3-(3-(4-(5-chloro-4-(((R)-1-(2,4-dichlorophenyl)ethyl)amino)pyrimidin-2-yl)-3,6-dihydropyridin-1(2H)-yl)piperidin-1-yl)propanoic acid

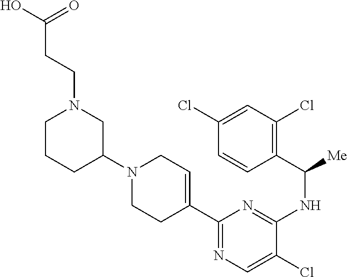 [SMILES: C[C@@H](Nc1nc(C2=CCN(C3CCCN(CCC(=O)O)C3)CC2)ncc1Cl)c1ccc(Cl)cc1Cl]

To a mixture of 5-chloro-N-((R)-1-(2,4-dichlorophenyl)ethyl)-2-(1-(piperidin-3-yl)-1,2,3,6-tetrahydropyridin-4-yl)pyrimidin-4-amine dihydrochloride (45 mg, 0.097 mmol) and N,N-diisopropylethylamine (52 mg, 0.40 mmol) in toluene (1 mL) was added acrylic acid (50 mg, 0.70 mmol). The mixture was stirred at 85° C. for 18 h. The mixture was concentrated in vacuo and purified by preparative HPLC to yield 3-(3-(4-(5-chloro-4-(((R)-1-(2,4-dichlorophenyl)ethyl)amino)pyrimidin-2-yl)-3,6-dihydropyridin-1(2H)-yl)piperidin-1-yl)propanoic acid. ¹H NMR (400 MHz, CD₃OD) δ 8.20 (s, 1H), 7.65-7.60 (m, 1H), 7.44 (s, 1H), 7.39 (d, J=8.6 Hz, 1H), 7.26 (d, J=8.6 Hz, 1H), 6.99-6.89 (m, 1H), 5.62 (q, J=7.9, 7.0 Hz, 1H), 3.89 (s, 2H), 3.65-3.53 (m, 1H), 3.53-3.33 (m, 6H), 3.19-3.08 (m, 1H), 2.93-2.75 (m, 4H), 2.74-2.60 (m, 1H), 2.22-2.03 (m, 2H), 1.90-1.74 (m, 2H), 1.56 (d, J=7.5 Hz, 3H). MS: (ES) m/z calculated for C₂₅H₃₁Cl₃N₅O₂ [M+H]⁺ 538.2, found 538.2.

### Example 23: Synthesis of 3-(3-(4-(5-chloro-4-(((R)-1-(2,4-dichlorophenyl)ethyl)amino)pyrimidin-2-yl)-3,6-dihydropyridin-1(2H)-yl)piperidin-1-yl)propanamide

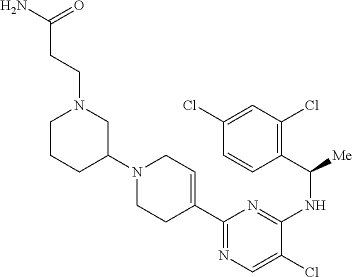 [SMILES: C[C@@H](Nc1nc(C2=CCN(C3CCCN(CCC(N)=O)C3)CC2)ncc1Cl)c1ccc(Cl)cc1Cl]

To a mixture of 5-chloro-N-((R)-1-(2,4-dichlorophenyl)ethyl)-2-(1-(piperidin-3-yl)-1,2,3,6-tetrahydropyridin-4-yl)pyrimidin-4-amine dihydrochloride (45 mg, 0.097 mmol), and potassium carbonate (54 mg, 0.4 mmol) in acetonitrile (0.5 mL) was added 3-bromopropanamide (18 mg, 0.12 mmol). The mixture was stirred at 80° C. for 5 h. The crude mixture was purified by preparative HPLC to yield 3-(3-(4-(5-chloro-4-(((R)-1-(2,4-dichlorophenyl)ethyl)amino)-pyrimidin-2-yl)-3,6-dihydropyridin-1(2H)-yl)piperidin-1-yl)propanamide. ¹H NMR (400 MHz, CD₃OD) δ 8.21 (s, 1H), 7.68-7.59 (m, 3H), 7.44 (s, 1H), 7.39 (d, J=8.7 Hz, 1H), 7.26 (d, J=8.3 Hz, 1H), 6.95 (d, J=2.0 Hz, 1H), 5.69-5.57 (m, 1H), 4.02-3.87 (m, 2H), 3.81-3.67 (m, 1H), 3.65-3.53 (m, 1H), 3.52-3.32 (m, 6H), 3.28-3.17 (m, 1H), 3.06-2.81 (m, 2H), 2.79-2.62 (m, 2H), 2.35-2.05 (m, 2H), 1.94-1.79 (m, 2H), 1.57 (d, J=7.3 Hz, 3H). MS: (ES) m/z calculated for C₂₅H₃₂Cl₃N₆O [M+H]⁺ 537.2, found 537.2.

### Example 24: Synthesis of 5-chloro-N-[(1R)-1-(2,4-dichlorophenyl)ethyl]-2-[4-(3-piperidyl)piperazin-1-yl]pyrimidin-4-amine

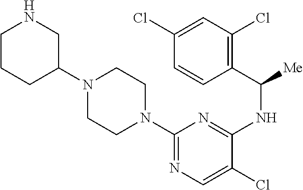

Step a: To a mixture of 2,5-dichloro-N-[(1R)-1-(2,4-dichlorophenyl)ethyl]pyrimidin-4-amine (500 mg, 1.5 mmol) and N,N-diisopropylethylamine (210 mg, 1.6 mmol) in DMF (5 mL) was added tert-butyl piperazine-1-carboxylate. The mixture was stirred at 80° C. for 3 h. The contents were cooled to room temperature and diluted with ethyl acetate, then washed with H₂O and brine. The organic layer was dried over MgSO₄, filtered, and concentrated. The crude residue was purified by silica gel column chromatography to yield tert-butyl 4-[5-chloro-4-[[(1R)-1-(2,4-dichlorophenyl)ethyl]amino]pyrimidin-2-yl]piperazine-1-carboxylate. MS: (ES) m/z calculated for C₂₁H₂₇Cl₃N₅O₂ [M+H]⁺ 486.1, found 486.1.

Step b: To a mixture of tert-butyl 4-[5-chloro-4-[[(1R)-1-(2,4-dichlorophenyl)ethyl]amino]pyrimidin-2-yl]piperazine-1-carboxylate (540 mg, 1.1 mmol) in DCM (4 mL) was added trifluoroacetic acid (1.5 g, 13 mmol). The contents were stirred at room temperature for 18 h. The mixture was quenched with saturated K₂CO₃ (aq) and extracted with DCM. The combined organic layers were dried over MgSO₄, filtered, and concentrated to yield 5-chloro-N-[(1R)-1-(2,4-dichlorophenyl)ethyl]-2-piperazin-1-yl-pyrimidin-4-amine. MS: (ES) m/z calculated for C₁₆H₁₉Cl₃N₅ [M+H]⁺ 386.1, found 386.1.

Step c: To a solution of 5-chloro-N-[(1R)-1-(2,4-dichlorophenyl)ethyl]-2-piperazin-1-yl-pyrimidin-4-amine (340 mg, 0.88 mmol), tert-butyl 3-oxopiperidine-1-carboxylate (180 mg, 0.89 mmol), triethylamine (180 mg, 1.8 mmol), and acetic acid (110 mg, 1.8 mmol) was added sodium cyanoborohydride (220 mg, 3.6 mmol). The mixture was stirred at room temperature for 24 h and then heated at 50° C. for 18 h. The crude reaction mixture was concentrated and purified by silica gel column chromatography to yield tert-butyl 3-[4-[5-chloro-4-[[(1R)-1-(2,4-dichlorophenyl)ethyl]amino]pyrimidin-2-yl]piperazin-1-yl]piperidine-1-carboxylate. MS: (ES) m/z calculated for C₂₆H₃₆Cl₃N₆O₂ [M+H]⁺ 569.2, found 569.2.

Step d: A mixture of tert-butyl 3-[4-[5-chloro-4-[[(1R)-1-(2,4-dichlorophenyl)ethyl]amino]pyrimidin-2-yl]piperazin-1-yl]piperidine-1-carboxylate (140 mg, 0.24 mmol) in a solution of 4 N HCl in dioxane (5 mL, 20 mmol) was stirred at room temperature for 18 h. The mixture was concentrated to dryness to yield 5-chloro-N-[(1R)-1-(2,4-dichlorophenyl)ethyl]-2-[4-(3-piperidyl)piperazin-1-yl]pyrimidin-4-amine. ¹H NMR (400 MHz, CD₃OD) δ 8.04 (s, 1H), 7.67-7.61 (m, 1H), 7.52 (s, 1H), 7.43 (dd, J=49.0, 8.5 Hz, 1H), 7.35 (d, J=9.2 Hz, 1H), 5.65 (q, J=7.1 Hz, 1H), 4.19-3.33 (m, 11H), 3.24-2.90 (m, 3H), 2.37-2.24 (m, 1H), 2.19-2.07 (m, 1H), 1.94-1.81 (m, 2H), 1.61 (d, J=7.1 Hz, 3H). MS: (ES) m/z calculated for C₂₁H₂₈Cl₃N₆ [M+H]⁺ 469.1, found 469.1.

### Example 25: Synthesis of 3-[3-[4-[5-chloro-4-[[(1R)-1-(2,4-dichlorophenyl)ethyl]amino]pyrimidin-2-yl]piperazin-1-yl]-1-piperidyl]propanoic acid

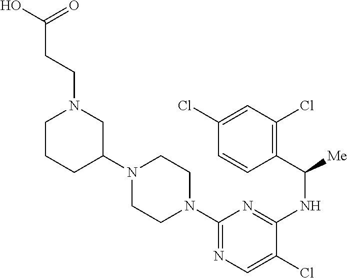 [SMILES: C[C@@H](Nc1nc(N2CCN(C3CCCN(CCC(=O)O)C3)CC2)ncc1Cl)c1ccc(Cl)cc1Cl]

To a mixture of 5-chloro-N-[(1R)-1-(2,4-dichlorophenyl)ethyl]-2-[4-(3-piperidyl)piperazin-1-yl]pyrimidin-4-amine dihydrochloride (48 mg, 0.088 mmol) and N,N-diisopropylethylamine (46 mg, 0.35 mmol) in toluene (1 mL) was added acrylic acid (45 mg, 0.62 mmol). The mixture was stirred at 85° C. for 18 h. The reaction was quenched with saturated NaHCO₃ (aq) and extracted with DCM. The combined organic layers were dried over MgSO₄, filtered, and concentrated in vacuo. The residue was purified by preparative HPLC to yield 3-[3 [4-[5-chloro-4-[[(1R)-1-(2,4-dichlorophenyl)ethyl]amino]pyrimidin-2-yl]piperazin-1-yl]-1-piperidyl]propanoic acid. ¹H NMR (400 MHz, CD₃OD) δ 8.35-8.28 (m, 1H), 7.90 (s, 1H), 7.51-7.44 (m, 1H), 7.42-7.36 (m, 1H), 7.35-7.28 (m, 1H), 5.57 (q, J=7.0 Hz, 1H), 3.76-3.60 (m, 4H), 3.49-3.34 (m, 4H), 3.25-3.00 (m, 2H), 2.93-2.81 (m, 3H), 2.79-2.59 (m, 4H), 2.12-1.99 (m, 1H), 1.99-1.87 (m, 1H), 1.84-1.66 (m, 2H), 1.58 (d, J=7.1 Hz, 3H). MS: (ES) m/z calculated for C₂₄H₃₂Cl₃N₆O₂ [M+H]⁺ 541.2, found 541.2.

### Example 26: Synthesis of 2-([3,4′-bipiperidin]-1′-yl)-5-chloro-N-((R)-1-(2,4-dichlorophenyl)ethyl)pyrimidin-4-amine

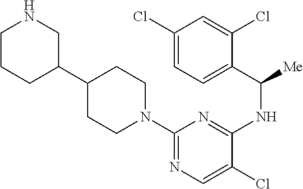 [SMILES: C[C@@H](Nc1nc(N2CCC(C3CCCNC3)CC2)ncc1Cl)c1ccc(Cl)cc1Cl]

Step a: To a solution of tert-butyl 5-(4,4,5,5-tetramethyl-1,3,2-dioxaborolan-2-yl)-3,6-dihydropyridine-1(2H)-carboxylate (400 mg, 1.29 mmol) and 4-iodopyridine (265 mg, 1.29 mmol) in ethanol (2 mL) and toluene (4 mL) was added an aqueous solution of 2M K₂CO₃ (2 mL, 3.88 mmol). The mixture was degassed with N₂ for 10 min and tetrakis(triphenylphosine)palladium(0) (74 mg, 0.065 mmol) was added. The reaction was heated at 100° C. for 3 h and the contents were then filtered through Celite. The filtrate was extracted with ethyl acetate and the combined organic layers were dried with sodium sulfate, filtered and concentrated. The crude material was purified by silica gel column chromatography to give tert-butyl 5,6-dihydro-[3,4′-bipyridine]-1(2H)-carboxylate. MS: (ES) m/z calculated for C₁₅H₂₁N₂O₂ [M+H]⁺ 261.2, found 261.2.

Step b: To a solution of tert-butyl 5,6-dihydro-[3,4′-bipyridine]-1(2H)-carboxylate (136 mg, 0.52 mmol) in MeOH (2 mL) was added PtO₂ (54 mg, 0.24 mmol) and acetic acid (0.15 mL, 2.61 mmol). The reaction mixture was placed onto a Parr Shaker under H₂ at 50 psi for 3 d. The contents were filtered and the filtrate was concentrated to provide tert-butyl [3,4′-bipiperidine]-1-carboxylate. MS: (ES) m/z calculated for C₁₅H₂₉N₂O₂ [M+H]⁺ 269.2, found 269.2.

Step c: To a solution of tert-butyl [3,4′-bipiperidine]-1-carboxylate (140 mg, 0.52 mmol) and (R)-2,5-dichloro-N-(1-(2,4-dichlorophenyl)ethyl)pyrimidin-4-amine (176 mg, 0.52 mmol) in DMSO (1 mL) was added N,N-diisopropylamine (0.45 mL, 2.61 mmol) followed by cesium fluoride (79.2 mg, 0.52 mmol). The mixture was heated at 100° C. for 16 h. The reaction was quenched with water and the contents were extracted with ethyl acetate. The combined organic layers were dried with sodium sulfate, filtered and concentrated. The crude material was purified by silica gel column chromatography to produce tert-butyl 1′-(5-chloro-4-(((R)-1-(2,4-dichlorophenyl)ethyl)amino)pyrimidin-2-yl)-[3,4′-bipiperidine]-1-carboxylate. MS: (ES) m/z calculated for C₂₇H₃₇Cl₃N₅O₂ [M+H]⁺ 568.2, found 568.2.

Step d: To a solution of tert-butyl 1′-(5-chloro-4-(((R)-1-(2,4-dichlorophenyl)ethyl)amino)pyrimidin-2-yl)-[3,4′-bipiperidine]-1-carboxylate (140 mg, 0.52 mmol) in dioxane (2 mL) was added a solution of 4.0 M HCl in dioxane (2 mL, 8.0 mmol). The reaction mixture was stirred at room temperature for 2 h and was then concentrated to dryness. The crude material was purified on preparative HPLC to yield 2-([3,4′-bipiperidin]-1′-yl)-5-chloro-N-((R)-1-(2,4-dichlorophenyl)ethyl)pyrimidin-4-amine. ¹H NMR (400 MHz, CD₃OD) δ 7.90 (s, 1H), 7.50 (s, 1H), 7.38 (d, J=8.4, 1H), 7.32 (d, J=8.4 Hz, 1H), 5.56 (q, J=6.8 Hz, 1H), 4.35 (d, J=10.8 Hz, 1H), 4.25 (d, J=11.6 Hz, 1H), 3.37-3.32 (m, 2H), 3.05-2.78 (m, 3H), 2.75-2.64 (m, 1H), 2.00-1.84 (m, 2H), 1.84-1.64 (m, 3H), 1.59 (d, J=7.2 Hz, 3H), 1.57-1.49 (m, 2H), 1.32-1.17 (m, 2H), 0.91 (s, 1H). MS: (ES) m/z calculated for C₂₂H₂₉Cl₃N₅ [M+H]⁺ 468.1, found 486.2.

### Example 27: Synthesis of 2-(1′-(5-chloro-4-(((R)-1-(2,4-dichlorophenyl)ethyl)amino)pyrimidin-2-yl)-[3,4′-bipiperidin]-1-yl)ethan-1-ol

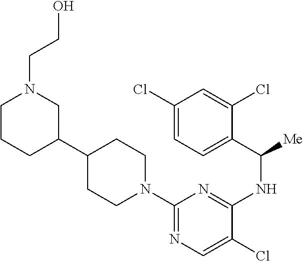 [SMILES: C[C@@H](Nc1nc(N2CCC(C3CCCN(CCO)C3)CC2)ncc1Cl)c1ccc(Cl)cc1Cl]

To a solution of 2-([3,4′-bipiperidin]-1′-yl)-5-chloro-N-((R)-1-(2,4-dichlorophenyl)ethyl)pyrimidin-4-amine (75 mg, 0.15 mmol) in MeCN (1 mL) was added K₂CO₃ (62 mg, 0.45 mmol) and 2-iodoethanol (44 mg, 0.26 mmol). The reaction was stirred at 80° C. for 4 h and then quenched with water. The contents were extracted with ethyl acetate and the combined organic layers were dried with sodium sulfate, filtered and concentrated. The crude material was purified by silica gel column chromatography followed by preparative HPLC to give 2-(1′-(5-chloro-4-(((R)-1-(2,4-dichlorophenyl)ethyl)amino)pyrimidin-2-yl)-[3,4′-bipiperidin]-1-yl)ethan-1-ol. ¹H NMR (400 MHz, CD₃OD) δ 7.86 (s, 1H), 7.48 (s, 1H), 7.37 (d, J=8.8 Hz, 1H), 7.31 (d, J=8.4 Hz, 1H), 5.67-5.46 (m, 1H), 4.46-4.26 (m, 2H), 3.89 (dd, J=5.3, 5.3 Hz, 2H), 3.59 (dd, J=14.0, 14.0 Hz, 2H), 3.28-3.16 (m, 2H), 2.96-2.82 (m, 3H), 2.74 (dd, J=12.3 Hz, 1H), 2.01 (d, J=15.2 Hz, 1H), 1.92-1.81 (m, 1H), 1.80-1.63 (m, 5H), 1.57 (d, J=6.8 Hz, 3H), 1.30-1.17 (m, 2H), 1.04-0.80 (m, 1H). MS: (ES) m/z calculated for C₂₄H₃₃Cl₃N₅O [M+H]⁺ 512.2, found 512.3.

### Example 28: Synthesis of 3-(1′-(5-chloro-4-(((R)-1-(2,4-dichlorophenyl)ethyl)amino)pyrimidin-2-yl)-[3,4′-bipiperidin]-1-yl)propanoic acid

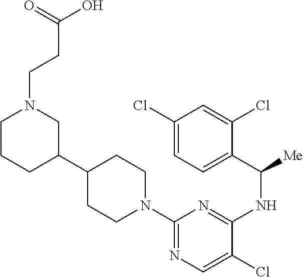 [SMILES: C[C@@H](Nc1nc(N2CCC(C3CCCN(CCC(=O)O)C3)CC2)ncc1Cl)c1ccc(Cl)cc1Cl]

To a solution of 2-([3,4′-bipiperidin]-1′-yl)-5-chloro-N-((R)-1-(2,4-dichlorophenyl)ethyl)pyrimidin-4-amine (75 mg, 0.15 mmol) in toluene (1 mL) was added N,N-diisopropylethylamine (0.05 mL, 0.74 mmol) and acrylic acid (0.02 mL, 0.26 mmol). The reaction was stirred at 85° C. for 16 h. The contents were concentrated and the crude material was purified by preparative HPLC to give 3-(1′-(5-chloro-4-(((R)-1-(2,4-dichlorophenyl)ethyl)amino)pyrimidin-2-yl)-[3,4′-bipiperidin]-1-yl)propanoic acid. ¹H NMR (400 MHz, CD₃OD) δ 7.89 (s, 1H), 7.50 (s, 1H), 7.37 (d, J=8.8 Hz, 1H), 7.32 (d, J=8.4 Hz, 1H), 5.61-5.53 (m, 1H), 4.36 (d, J=12.0 Hz, 1H), 4.31-4.23 (m, 1H), 3.60-3.46 (m, 3H), 3.45-3.37 (m, 2H), 2.99-2.89 (m, 3H), 2.89-2.81 (m, 2H), 2.80-2.70 (m, 1H), 2.07-1.98 (m, 1H), 1.91-1.66 (m, 5H), 1.59 (d, J=7.2, Hz, 3H), 1.32-1.15 (m, 2H), 1.06-0.85 (m, 1H). MS: (ES) m/z calculated for C₂₅H₃₃Cl₃N₅O₂ [M+H]⁺ 540.2, found 540.2.

### Example 29: Synthesis of (R)-N-((3R,4S)-1-(4-(((R)-1-(2,4-dichlorophenyl)ethyl)amino)-5-fluoropyrimidin-2-yl)-3-hydroxypiperidin-4-yl)pyrrolidine-2-carboxamide

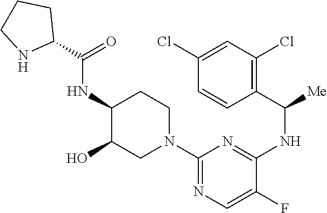 [SMILES: C[C@@H](Nc1nc(N2CC[C@H](NC(=O)[C@H]3CCCN3)[C@H](O)C2)ncc1F)c1ccc(Cl)cc1Cl]

Step a: To a solution of 2,4-dichloro-5-fluoropyrimidine (350 mg, 2.1 mmol) and (R)-1-(2,4-dichlorophenyl)ethan-1-amine (400 mg, 2.1 mmol) in DMSO (2 mL) was added DIPEA (1.8 mL, 10.5 mmol). The reaction mixture was stirred at 90° C. for 3 h. Water was added and the mixture was extracted with EtOAc. The combined organic layers were dried with sodium sulfate, filtered and concentrated. The crude material was purified by silica gel column chromatography to give (R)-2-chloro-N-(1-(2,4-dichlorophenyl)ethyl)-5-fluoropyrimidin-4-amine. MS: (ES) m/z calculated for C₁₂H₁₀Cl₃FN₃ [M+H]⁺ 320.0, found 320.0.

Step b: To a solution of (R)-2-chloro-N-(1-(2,4-dichlorophenyl)ethyl)-5-fluoropyrimidin-4-amine (100 mg, 0.31 mmol) and tert-butyl ((3R,4S)-3-hydroxypiperidin-4-yl)carbamate (77 mg, 0.35 mmol) in DMSO (1 mL) was added DIPEA (0.1 mL, 0.58 mmol). The reaction mixture was stirred at 100° C. for 16 h. Water was added and the mixture was extracted with EtOAc. The combined organic layers were dried with sodium sulfate, filtered and concentrated. The crude material was purified by silica gel column chromatography to give tert-butyl ((3R,4S)-1-(4-(((R)-1-(2,4-dichlorophenyl)ethyl)amino)-5-fluoropyrimidin-2-yl)-3-hydroxypiperidin-4-yl)carbamate. MS: (ES) m/z calculated for C₂₂H₂₉Cl₂FN₅O₃ [M+H]⁺ 500.2, found 500.2.

Step c: To a solution containing tert-butyl ((3R,4S)-1-(4-(((R)-1-(2,4-dichlorophenyl)ethyl)amino)-5-fluoropyrimidin-2-yl)-3-hydroxypiperidin-4-yl)carbamate (110 mg, 0.22 mmol) in dioxane (1 mL) was added 4.0 M HCl in dioxane (1 mL, 4.0 mmol). The contents were stirred at room temperature for 3 h and then concentrated to yield (3R,4S)-4-amino-1-(4-(((R)-1-(2,4-dichlorophenyl)ethyl)amino)-5-fluoropyrimidin-2-yl)piperidin-3-ol. MS: (ES) m/z calculated for C₁₇H₂₁Cl₂FN₅O [M+H]⁺ 400.1, found 400.1.

Step d: To a solution containing (3R,4S)-4-amino-1-(4-(((R)-1-(2,4-dichlorophenyl)ethyl)amino)-5-fluoropyrimidin-2-yl)piperidin-3-ol (85 mg, 0.19 mmol) and (tert-butoxycarbonyl)-D-proline (50 mg, 0.23 mmol) in DMF (1 mL) was added HATU (148 mg, 0.39 mmol) followed by Et₃N (0.11 mL, 0.79 mmol). The reaction was stirred at room temperature for 16 h and then quenched with water. The mixture was extracted with EtOAc and the combined organic layers were dried with sodium sulfate, filtered and concentrated. The crude material was purified by silica gel column chromatography to yield tert-butyl (R)-2-(((3R,4S)-1-(4-(((R)-1-(2,4-dichlorophenyl)ethyl)amino)-5-fluoropyrimidin-2-yl)-3-hydroxypiperidin-4-yl)carbamoyl)pyrrolidine-1-carboxylate. MS: (ES) m/z calculated for C₂₇H₃₆Cl₂FN₆O₄ [M+H]⁺ 597.2, found 597.3.

Step e: To a solution containing tert-butyl (R)-2-(((3R,4S)-1-(4-(((R)-1-(2,4-dichlorophenyl)ethyl)amino)-5-fluoropyrimidin-2-yl)-3-hydroxypiperidin-4-yl)carbamoyl)pyrrolidine-1-carboxylate (102 mg, 0.17 mmol) in dioxane (1 mL) was added 4.0 M HCl in dioxane (1 mL, 4.0 mmol). The contents were stirred at room temperature for 5 h, then concentrated and purified by preparative HPLC to yield (R)-N-((3R,4S)-1-(4-(((R)-1-(2,4-dichlorophenyl)ethyl)amino)-5-methyl-5H-pyrrolo[3,2-d]pyrimidin-2-yl)-3-hydroxypiperidin-4-yl)pyrrolidine-2-carboxamide. ¹H NMR (400 MHz, CD₃OD) δ 7.82 (dd, J=5.3, 0.7 Hz, 1H), 7.49 (d, J=2.1 Hz, 1H), 7.41 (d, J=8.4 Hz, 1H), 7.34 (dd, J=8.4, 2.1 Hz, 1H), 5.75-5.52 (m, 1H), 4.87-4.83 (m, 1H), 4.41 (s, 1H), 4.24 (dd, J=8.5, 6.6 Hz, 1H), 4.06-3.98 (m, 2H), 3.53-3.36 (m, 1H), 3.36-3.32 (m, 3H), 3.22-2.95 (m, 1H), 2.43 (ddd, J=12.7, 8.4, 6.2 Hz, 1H), 2.12-1.88 (m, 4H), 1.58 (d, J=7.1 Hz, 3H). MS: (ES) m/z calculated for C₂₂H₂₈Cl₂FN₆O₂ [M+H]⁺ 497.2, found 497.2.

### Example 30: Synthesis of (R)-5-(4-(4-(((R)-1-(2,4-dichlorophenyl)ethyl)amino)-5-fluoropyrimidin-2-yl)piperazine-1-carbonyl)pyrrolidin-2-one

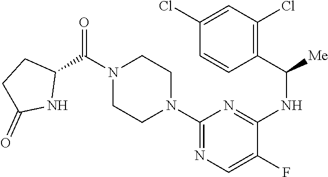 [SMILES: C[C@@H](Nc1nc(N2CCN(C(=O)[C@H]3CCC(=O)N3)CC2)ncc1F)c1ccc(Cl)cc1Cl]

Step a: To a solution of 2,4-dichloro-5-fluoropyrimidine (350 mg, 2.1 mmol) and (R)-1-(2,4-dichlorophenyl)ethan-1-amine (400 mg, 2.1 mmol) in DMSO (2 mL) was added DIPEA (1.8 mL, 10.5 mmol). The reaction mixture was stirred at 90° C. for 3 h. Water was added and the mixture was extracted with EtOAc. The combined organic layers were dried with sodium sulfate, filtered and concentrated. The crude material was purified by silica gel column chromatography to give (R)-2-chloro-N-(1-(2,4-dichlorophenyl)ethyl)-5-fluoropyrimidin-4-amine. MS: (ES) m/z calculated for C₁₂H₁₀Cl₃FN₃ [M+H]⁺ 320.0, found 320.0.

Step b: To a mixture of (R)-2-chloro-N-(1-(2,4-dichlorophenyl)ethyl)-5-fluoropyrimidin-4-amine (624 mg, 0.91 mmol) and tert-butyl piperazine-1-carboxylate (400 mg, 2.15 mmol) was added DIPEA (1.7 mL, 9.8 mmol). The reaction mixture was stirred at 115° C. for 3 h. An additional amount of tert-butyl piperazine-1-carboxylate (400 mg, 2.15 mmol) was added and the contents were stirred for an additional 16 h at 115° C. Water was added and the mixture was extracted with EtOAc. The combined organic layers were dried with sodium sulfate, filtered and concentrated. The crude material was purified by silica gel column chromatography to give tert-butyl (R)-4-(4-((1-(2,4-dichlorophenyl)ethyl)amino)-5-fluoropyrimidin-2-yl)piperazine-1-carboxylate. MS: (ES) m/z calculated for C₂₁H₂₇Cl₂FN₅O₂ [M+H]⁺ 470.1, found 470.1.

Step c: To a solution containing tert-butyl (R)-4-(4-((1-(2,4-dichlorophenyl)ethyl)amino)-5-fluoropyrimidin-2-yl)piperazine-1-carboxylate (150 mg, 0.32 mmol) in dioxane (1 mL) was added 4.0 M HCl in dioxane (1 mL, 4.0 mmol). The contents were stirred at room temperature for 3 h and then concentrated to yield (R)-N-(1-(2,4-dichlorophenyl)ethyl)-5-fluoro-2-(piperazin-1-yl)pyrimidin-4-amine hydrochloride. MS: (ES) m/z calculated for C₁₆H₁₉Cl₂FN₅ [M+H]⁺ 370.1, found 370.1.

Step d: To a solution containing (R)-N-(1-(2,4-dichlorophenyl)ethyl)-5-fluoro-2-(piperazin-1-yl)pyrimidin-4-amine hydrochloride (130 mg, 0.32 mmol) and (R)-5-oxopyrrolidine-2-carboxylic acid (51 mg, 0.40 mmol) in DMF (0.5 mL) was added HATU (242 mg, 0.64 mmol) followed by Et₃N (0.15 mL, 1.1 mmol). The reaction was stirred at room temperature for 16 h and then quenched with water. The mixture was extracted with EtOAc and the combined organic layers were dried with sodium sulfate, filtered and concentrated. The crude material was purified by silica gel column chromatography to yield (R)-5-(4-(4-(((R)-1-(2,4-dichlorophenyl)ethyl)amino)-5-fluoropyrimidin-2-yl)piperazine-1-carbonyl)pyrrolidin-2-one. ¹H NMR (400 MHz, CD₃OD) δ 7.99-7.87 (m, 1H), 7.52 (s, 1H), 7.42 (d J=8.6 Hz, 1H), 7.34 (d, J=8.4 Hz, 1H), 5.73-5.55 (m, 1H), 4.74 (dt, J=8.0, 3.4 Hz, 1H), 3.94-3.48 (m, 8H), 2.57-2.46 (m, 1H), 2.40-2.34 (m, 2H), 2.09-1.99 (m, 1H), 1.59 (d, J=7.0 Hz, 3H). MS: (ES) m/z calculated for C₂₁H₂₄Cl₂FN₆O₂ [M+H]⁺ 481.1, found 481.1.

### Example 31: Synthesis of (5R)-5-(4-(5-chloro-4-((1-(2,4-dichloro-5-fluorophenyl)ethyl)amino)pyrimidin-2-yl)piperazine-1-carbonyl)pyrrolidin-2-one

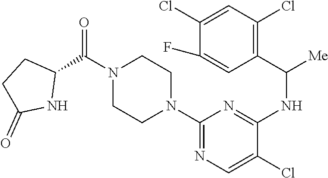 [SMILES: CC(Nc1nc(N2CCN(C(=O)[C@H]3CCC(=O)N3)CC2)ncc1Cl)c1cc(F)c(Cl)cc1Cl]

Step a: To a vial was added 2,4,5-trichloropyrimidine (91 mg, 0.50 mmol), DMF (4 mL), 1-(2,4-dichloro-5-fluoro-phenyl)ethanamine (208 mg, 1.00 mmol) and DIPEA (0.35 mL, 2.00 mmol). The mixture was stirred at 100° C. for 1.5 h, cooled to room temperature, diluted with water and sat. NaHCO₃ and extracted with EtOAc. The organic layer was separated, filtered over Na₂SO₄, concentrated and purified by silica gel column chromatography to yield 2,5-dichloro-N-[1-(2,4-dichloro-5-fluoro-phenyl)ethyl]pyrimidin-4-amine. MS: (ES) m/z calculated for C₁₂H₉Cl₄FN₃ [M+H]⁺ 354.0, found 354.0.

Step b: To a vial of 2,5-dichloro-N-[1-(2,4-dichloro-5-fluoro-phenyl)ethyl]pyrimidin-4-amine (100 mg, 0.28 mmol) was added tert-butyl piperazine-1-carboxylate (1.00 g, 5.37 mmol). The mixture was stirred at 125° C. for 1 h, cooled to room temperature and purified by silica gel column chromatography to yield tert-butyl 4-[5-chloro-4-[1-(2,4-dichloro-5-fluoro-phenyl)ethylamino]pyrimidin-2-yl]piperazine-1-carboxylate. MS: (ES) m/z calculated for C₂₁H₂₆Cl₃FN₅O₂ [M+H]⁺ 504.1, found 504.1.

Step c: To a flask containing tert-butyl 4-[5-chloro-4-[1-(2,4-dichloro-5-fluoro-phenyl)ethylamino]pyrimidin-2-yl]piperazine-1-carboxylate (120 mg, 0.24 mmol) was added 4.0 M HCl in dioxane (3.0 mL, 12.0 mmol). The mixture was stirred for 0.5 h and then concentrated to dryness to yield 5-chloro-N-[1-(2,4-dichloro-5-fluoro-phenyl)ethyl]-2-piperazin-1-yl-pyrimidin-4-amine hydrochloride. MS: (ES) m/z calculated for C₁₆H₁₈Cl₃FN₅ [M+H]⁺ 404.1, found 404.0.

Step d: To a vial of 5-chloro-N-[1-(2,4-dichloro-5-fluoro-phenyl)ethyl]-2-piperazin-1-yl-pyrimidin-4-amine hydrochloride (35 mg, 0.079 mmol) in DMF (1 mL) was added (2R)-5-oxopyrrolidine-2-carboxylic acid (25 mg, 0.19 mmol), HATU (70 mg, 0.18 mmol), and Et₃N (0.10 mL, 0.72 mmol). The mixture was stirred for 0.5 h, quenched with water and purified by preparative HPLC to yield (5R)-5-[4-[5-chloro-4-[1-(2,4-dichloro-5-fluoro-phenyl)ethylamino]pyrimidin-2-yl]piperazine-1-carbonyl]pyrrolidin-2-one. ¹H NMR (400 MHz, CD₃OD) δ 7.98 (s, 1H), 7.67-7.62 (m, 1H), 7.32 (dd, J=10, 1.2 Hz, 1H), 5.63-5.55 (m, 1H), 4.73-4.67 (m, 1H), 3.80-3.59 (m, 7H), 3.57-3.47 (m, 1H), 2.57-2.45 (m, 1H), 2.42-2.31 (m, 2H), 2.07-1.94 (m, 1H), 1.60 (d, J=7.2 Hz, 3H). MS: (ES) m/z calculated for C₂₁H₂₃Cl₃FN₆O₂ [M+H]⁺ 515.1, found 515.1.

### Example 32: Synthesis of 3-(1′-(5-chloro-4-(((R)-1-(2,4-dichlorophenyl)ethyl)amino)pyrimidin-2-yl)-[3,4′-bipiperidin]-1-yl)propanamide

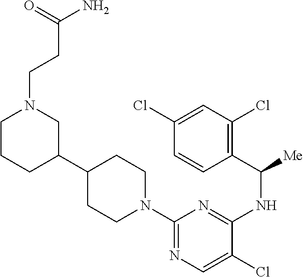 [SMILES: C[C@@H](Nc1nc(N2CCC(C3CCCN(CCC(N)=O)C3)CC2)ncc1Cl)c1ccc(Cl)cc1Cl]

To a vial of 3-(1′-(5-chloro-4-(((R)-1-(2,4-dichlorophenyl)ethyl)amino)pyrimidin-2-yl)-[3,4′-bipiperidin]-1-yl)propanoic acid (32 mg, 0.058 mmol) in DCM (4 mL) was added oxalyl dichloride (50 mg, 0.39 mmol) and DMF (0.05 mL). The mixture was stirred for 15 m and then concentrated to dryness. The obtained residue was redissolved in DCM and a solution of sat. NH₃ in DCM (0.3 mL) was added. The mixture was stirred for 0.5 h and purified by silica gel column chromatography to obtain 3-(1′-(5-chloro-4-(((R)-1-(2,4-dichlorophenyl)ethyl)amino)pyrimidin-2-yl)-[3,4′-bipiperidin]-1-yl)propanamide. ¹H NMR (400 MHz, CD₃OD) δ 7.86 (s, 1H), 7.75 (s, 1H), 7.74 (s, 1H), 7.36 (m, 1H), 7.27 (m, 1H), 5.47-5.40 (m, 1H), 4.45 (d, J=12.8 Hz, 2H), 3.76-3.69 (m, 1H), 3.55-3.20 (m, 5H), 2.97-2.60 (m, 6H), 2.02-1.92 (m, 1H), 1.85-1.74 (m, 2H), 1.65-1.57 (m, 1H), 1.57-1.45 (m, 4H), 1.39 (d, J=6.4 Hz, 3H), 1.30-1.05 (m, 2H); MS: (ES) m/z calculated for C₂₅H₃₄Cl₃N₆O [M+H]⁺ 539.2, found 539.2.

### Example 33: Synthesis of 2-(4-(D-prolyl)piperazin-1-yl)-4-(((R)-1-(2,4-dichlorophenyl)ethyl)amino)-5-hydroxypyrimidine

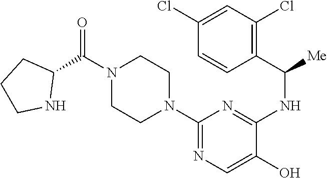 [SMILES: C[C@@H](Nc1nc(N2CCN(C(=O)[C@H]3CCCN3)CC2)ncc1O)c1ccc(Cl)cc1Cl]

To a vial of [4-[4-[[(1R)-1-(2,4-dichlorophenyl)ethyl]amino]-5-methoxy-pyrimidin-2-yl]piperazin-1-yl]-[(2R)-pyrrolidin-2-yl]methanone hydrochloride (50 mg, 0.10 mmol) was added BBr₃ (0.13 mL, 1.37 mmol). The mixture was stirred for 3 h and then cooled to 0° C. and water and MeOH were added. The contents were concentrated to dryness and purified by preparative HPLC to obtain 2-(4-(D-prolyl)piperazin-1-yl)-4-(((R)-1-(2,4-dichlorophenyl)ethyl)amino)-5-hydroxypyrimidine. ¹H NMR (400 MHz, CD₃OD) δ 7.49 (s, 1H), 7.42 (d, J=8.4 Hz, 1H), 7.31 (d, J=8.0 Hz, 1H), 7.12 (s, 1H), 5.63 (q, J=7.2 Hz, 1H), 5.49 (s, 1H), 4.71 (t, J=7.6 Hz, 1H), 3.83-3.32 (m, 10H), 2.56-2.46 (m, 1H), 2.14-1.90 (m, 3H), 1.57 (d, J=6.8 Hz, 3H). MS: (ES) m/z calculated for C₂₁H₂₇C₁₂N₆O₂ [M+H]⁺ 465.1, found 465.1.

### Example 34: Synthesis of (R)-N-((3S,4S)-1-(4-(((R)-1-(2,4-dichlorophenyl)ethyl)amino)-5-methoxypyrimidin-2-yl)-3-hydroxypiperidin-4-yl)pyrrolidine-2-carboxamide

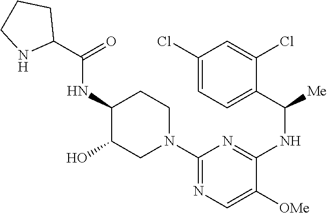 [SMILES: COc1cnc(N2CC[C@H](NC(=O)C3CCCN3)[C@@H](O)C2)nc1N[C@H](C)c1ccc(Cl)cc1Cl]

Step a: To a vial of 2-chloro-N-[(1R)-1-(2,4-dichlorophenyl)ethyl]-5-methoxy-pyrimidin-4-amine (250 mg, 0.75 mmol) was added tert-butyl N-[(3S,4S)-3-hydroxy-4-piperidyl]carbamate (325 mg, 1.50 mmol). The mixture was stirred at 125° C. for 3 h, cooled to room temperature and purified by silica gel column chromatography to yield tert-butyl N-[(3S,4S)-1-[4-[[(1R)-1-(2,4-dichlorophenyl)ethyl]amino]-5-methoxy-pyrimidin-2-yl]-3-hydroxy-4-piperidyl]carbamate. MS: (ES) m/z calculated for C₂₃H₃₂C₁₂N₅O₄ [M+H]⁺ 512.2, found 512.3.

Step b: To a vial was added tert-butyl N-[(3S,4S)-1-[4-[[(1R)-1-(2,4-dichlorophenyl)ethyl]amino]-5-methoxy-pyrimidin-2-yl]-3-hydroxy-4-piperidyl]carbamate (380 mg, 0.74 mmol) and 4.0 M HCl in dioxane (3.0 mL, 12.0 mmol). The mixture was stirred for 0.5 h and concentrated to dryness to yield (3S,4S)-4-amino-1-[4-[[(1R)-1-(2,4-dichlorophenyl)ethyl]amino]-5-methoxy-pyrimidin-2-yl]piperidin-3-ol hydrochloride. MS: (ES) m/z calculated for C₁₈H₂₄Cl₂N₅O₂ [M+H]⁺ 412.1, found 412.1.

Step c: To a vial of (3S,4S)-4-amino-1-[4-[[(1R)-1-(2,4-dichlorophenyl)ethyl]amino]-5-methoxy-pyrimidin-2-yl]piperidin-3-ol hydrochloride (40 mg, 0.089 mmol) in DMF (2 mL) was added (2R)-1-tert-butoxycarbonylpyrrolidine-2-carboxylic acid (28 mg, 0.130 mmol), HATU (70 mg, 0.18 mmol), and Et₃N (0.10 mL, 0.72 mmol). The mixture was stirred for 0.5 h, quenched with water and sat. NaHCO₃ and extracted with EtOAc. The organic layer was collected, filtered through Na₂SO₄ and purified by silica gel column chromatography to yield tert-butyl (2R)-2-[[(3S,4S)-1-[4-[[(1R)-1-(2,4-dichlorophenyl)ethyl]amino]-5-methoxy-pyrimidin-2-yl]-3-hydroxy-4-piperidyl]carbamoyl]pyrrolidine-1-carboxylate. MS: (ES) m/z calculated for C₂₈H₃₉C₁₂N₆O₅ [M+H]⁺ 609.2, found 609.3.

Step d: To a vial containing tert-butyl (2R)-2-[[(3S,4S)-1-[4-[[(1R)-1-(2,4-dichlorophenyl)ethyl]amino]-5-methoxy-pyrimidin-2-yl]-3-hydroxy-4-piperidyl]carbamoyl]pyrrolidine-1-carboxylate (50 mg, 0.084 mmol) was added 4 N HCl in dioxane (2.0 mL, 8.19 mmol). The mixture was stirred for 0.5 h, concentrated to dryness and purified by preparative HPLC to yield the desired product (R)-N-((3S,4S)-1-(4-(((R)-1-(2,4-dichlorophenyl)ethyl)amino)-5-methoxypyrimidin-2-yl)-3-hydroxypiperidin-4-yl)pyrrolidine-2-carboxamide. ¹H NMR (400 MHz, CD₃OD) δ 8.92 (d, J=6.0 Hz, 1H), 8.46 (d, J=7.6 Hz, 1H), 7.46 (s, 1H), 7.38 (d, J=8.8 Hz, 1H), 7.30 (d, J=8.0 Hz, 1H), 7.24 (s, 1H), 5.64-5.54 (m, 1H), 4.26-4.16 (m, 3H), 3.89 (s, 3H), 3.87-3.80 (m, 1H), 3.50-3.32 (m, 3H), 3.17 (dd, J=11.4, 11.4 Hz, 1H), 3.00 (dd, J=11.8, 11.8 Hz, 1H), 2.47-2.37 (m, 1H), 2.10-1.90 (m, 4H), 1.56 (d, J=7.2 Hz, 3H), 1.30-1.18 (m, 1H). MS: (ES) m/z calculated for C₂₃H₃₁C₁₂N₆O₃ [M+H]⁺ 509.2, found 509.2.

### Example 35: Synthesis of (R)-5-(4-(4-(((R)-1-(2,4-dichlorophenyl)ethyl)amino)-5-methoxypyrimidin-2-yl)piperazine-1-carbonyl)pyrrolidin-2-one

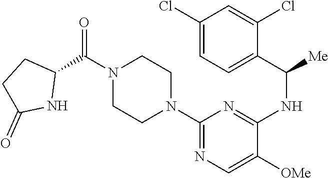 [SMILES: COc1cnc(N2CCN(C(=O)[C@H]3CCC(=O)N3)CC2)nc1N[C@H](C)c1ccc(Cl)cc1Cl]

To a vial of N-[(1R)-1-(2,4-dichlorophenyl)ethyl]-5-methoxy-2-piperazin-1-yl-pyrimidin-4-amine hydrochloride (35 mg, 0.084 mmol) in DMF (1.5 mL) was added (2R)-5-oxopyrrolidine-2-carboxylic acid (25 mg, 0.19 mmol), HATU (85 mg, 0.22 mmol), and Et₃N (0.10 mL, 0.72 mmol). The mixture was stirred for 0.5 h, quenched with water and purified by preparative HPLC to yield (R)-5-(4-(4-(((R)-1-(2,4-dichlorophenyl)ethyl)amino)-5-methoxypyrimidin-2-yl)piperazine-1-carbonyl)pyrrolidin-2-one. ¹H NMR (400 MHz, CD₃OD) δ 8.98 (d, J=7.2 Hz, 1H), 7.50 (s, 1H), 7.39 (d, J=8.4 Hz, 1H), 7.31 (d, J=8.4 Hz, 1H), 7.28 (s, 1H), 5.67-5.59 (m, 1H), 4.74-4.68 (m, 1H), 3.91 (s, 3H), 3.80-3.51 (m, 8H), 2.55-2.45 (m, 1H), 2.45-2.25 (m, 2H), 2.07-1.97 (m, 1H), 1.57 (d, J=6.4 Hz, 3H). MS: (ES) m/z calculated for C₂₂H₂₇C₁₂N₆O₃ [M+H]⁺ 493.1, found 493.1.

### Example 36: Synthesis of 2-(4-(D-prolyl)piperazin-1-yl)-4-(((R)-1-(2,4-dichlorophenyl)ethyl)amino)-5-methoxypyrimidine

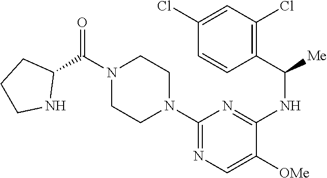 [SMILES: COc1cnc(N2CCN(C(=O)[C@H]3CCCN3)CC2)nc1N[C@H](C)c1ccc(Cl)cc1Cl]

Step a: To a vial of 2,4-dichloro-5-methoxy-pyrimidine (754 mg, 4.21 mmol) in DMF (12 mL) was added (1R)-1-(2,4-dichlorophenyl)ethanamine (800 mg, 4.21 mmol) and DIPEA (0.77 mL, 4.42 mmol). The mixture was stirred at 90° C. for 5 h, cooled to room temperature, diluted with water and sat. NaHCO₃ and extracted with EtOAc. The organic layer was separated, filtered over Na₂SO₄, concentrated to dryness and purified by silica gel column chromatography to yield 2-chloro-N-[(1R)-1-(2,4-dichlorophenyl)ethyl]-5-methoxy-pyrimidin-4-amine. MS: (ES) m/z calculated for C₁₃H₁₃Cl₃N₃O [M+H]⁺ 332.0, found 332.0.

Step b: To a vial was added 2-chloro-N-[(1R)-1-(2,4-dichlorophenyl)ethyl]-5-methoxy-pyrimidin-4-amine (200 mg, 0.60 mmol) and tert-butyl piperazine-1-carboxylate (1.00 g, 5.37 mmol). The mixture was stirred at 125° C. for 5 h, cooled to room temperature and purified by silica gel column chromatography to yield tert-butyl 4-[4-[[(1R)-1-(2,4-dichlorophenyl)ethyl]amino]-5-methoxy-pyrimidin-2-yl]piperazine-1-carboxylate. MS: (ES) m/z calculated for C₂₂H₃₀C₁₂N₅₀₃ [M+H]⁺ 482.2, found 482.1.

Step c: To a flask containing tert-butyl 4-[4-[[(1R)-1-(2,4-dichlorophenyl)ethyl]amino]-5-methoxy-pyrimidin-2-yl]piperazine-1-carboxylate (280 mg, 0.58 mmol) was added 4.0 M HCl in dioxane (4.0 mL, 16.0 mmol). The mixture was stirred for 0.5 h and then concentrated to dryness to yield N-[(1R)-1-(2,4-dichlorophenyl)ethyl]-5-methoxy-2-piperazin-1-yl-pyrimidin-4-amine hydrochloride. MS: (ES) m/z calculated for C₁₇H₂₂C₁₂N₅O [M+H]⁺ 382.1, found 382.1.

Step d: To a vial of N-[(1R)-1-(2,4-dichlorophenyl)ethyl]-5-methoxy-2-piperazin-1-yl-pyrimidin-4-amine (170 mg, 0.44 mmol) in DMF (2 mL) was added (2R)-1-tert-butoxycarbonylpyrrolidine-2-carboxylic acid (110 mg, 0.51 mmol), HATU (200 mg, 0.53 mmol), and Et₃N (0.30 mL, 2.15 mmol). The mixture was stirred for 0.5 h, quenched with water and sat. NaHCO₃ and extracted with EtOAc. The organic layer was collected, filtered through Na₂SO₄ and purified by silica gel column chromatography to yield tert-butyl (2R)-2-[4-[4-[[(1R)-1-(2,4-dichlorophenyl)ethyl]amino]-5-methoxy-pyrimidin-2-yl]piperazine-1-carbonyl]pyrrolidine-1-carboxylate. MS: (ES) m/z calculated for C₂₇H₃₇C₁₂N₆O₄ [M+H]⁺ 579.2, found 579.3.

Step e: To a vial of tert-butyl (2R)-2-[4-[4-[[(1R)-1-(2,4-dichlorophenyl)ethyl]amino]-5-methoxy-pyrimidin-2-yl]piperazine-1-carbonyl]pyrrolidine-1-carboxylate (160 mg, 0.28 mmol) was added 4.0 M HCl in dioxane (4.0 mL, 16.0 mmol). The mixture was stirred for 0.5 h, evaporated to dryness, basified with sat. NaHCO₃ and extracted with EtOAc. The organic layer was separated, filtered over Na₂SO₄, concentrated and purified by silica gel column chromatography The to obtain [4-[4-[[(1R)-1-(2,4-dichlorophenyl)ethyl]amino]-5-methoxy-pyrimidin-2-yl]piperazin-1-yl]-[(2R)-pyrrolidin-2-yl]methanone. ¹H NMR (400 MHz, CD₃OD) δ 9.01 (d, J=6.8 Hz, 1H), 7.50 (s, 1H), 7.40 (d, J=8.4 Hz, 1H), 7.32 (d, J=8.4 Hz, 1H), 7.29 (s, 1H), 5.67-5.59 (m, 1H), 4.73 (dd, J=8.6 Hz, 1H), 3.92 (s, 3H), 3.86-3.33 (m, 10H), 2.55-2.43 (m, 1H), 2.05-1.90 (m, 3H), 1.57 (d, J=6.8 Hz, 3H). MS: (ES) m/z calculated for C₂₂H₂₉C₁₂N₆O₂ [M+H]⁺ 479.2, found 479.3.

### Example 37: Synthesis of 2-(4-(D-prolyl)piperazin-1-yl)-4-(((R)-1-(2,4-dichlorophenyl)ethyl)amino)-5-(2,2,2-trifluoroethoxy)pyrimidine

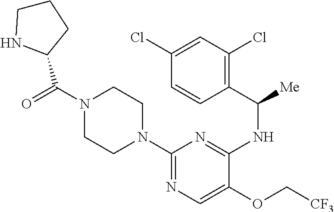 [SMILES: C[C@@H](Nc1nc(N2CCN(C(=O)[C@H]3CCCN3)CC2)ncc1OCC(F)(F)F)c1ccc(Cl)cc1Cl]

Step a: To a solution of 2,4-dichloropyrimidin-5-ol (1.00 g, 6.06 mmol) and cesium carbonate (2.57 g, 7.88 mmol) in DMF (6 mL) was added 2,2,2-trifluoroethyl trifluoromethanesulfonate (1.69 g, 7.27 mmol). The reaction mixture was stirred at room temperature for 4 h and was then diluted with water and ethyl acetate. The organic and aqueous layers were separated and the aqueous layer was extracted with EtOAc. The combined organic layers were dried with sodium sulfate, filtered and concentrated. The crude material was purified by silica gel column chromatography to provide 2,4-dichloro-5-(2,2,2-trifluoroethoxy)pyrimidine. MS: (ES) m/z calculated for C₆H₄C₁₂F₃N₂O [M+H]⁺ 247.0, found 246.9.

Step b: To a solution of 2,4-dichloro-5-(2,2,2-trifluoroethoxy)pyrimidine (590 mg, 2.39 mmol) and (R)-1-(2,4-dichlorophenyl)ethan-1-amine (450 mg, 2.39 mmol) in DMF (8 mL) was added DIPEA (1.2 mL, 7.2 mmol). The reaction mixture was heated at 100° C. for 4 h and was then diluted with water and ethyl acetate. The organic and aqueous layers were separated and the aqueous layer was extracted with ethyl acetate. The combined organic layers were dried with sodium sulfate, filtered and concentrated. The crude material was purified by silica gel column chromatography to provide (R)-2-chloro-N-(1-(2,4-dichlorophenyl)ethyl)-5-(2,2,2-trifluoroethoxy)pyrimidin-4-amine. MS: (ES) m/z calculated for C₁₄H₁₂Cl₃F₃N₃O [M+H]⁺ 400.0, found 400.0.

Step c: To a solution of (R)-2-chloro-N-(1-(2,4-dichlorophenyl)ethyl)-5-(2,2,2-trifluoroethoxy)pyrimidin-4-amine (500 mg, 1.25 mmol) in DMSO (3 mL) was added tert-butyl piperazine-1-carboxylate (2.10 g, 11.2 mmol). The reaction mixture was heated at 120° C. for 16 h and was then diluted with water and ethyl acetate. The organic and aqueous layers were separated and the aqueous layer was extracted with ethyl acetate. The combined organic layers were dried with sodium sulfate, filtered and concentrated. The crude material was purified by silica gel column chromatography to provide tert-butyl (R)-4-(4-((1-(2,4-dichlorophenyl)ethyl)amino)-5-(2,2,2-trifluoroethoxy)pyrimidin-2-yl)piperazine-1-carboxylate. MS: (ES) m/z calculated for C₂₃H₂₉C₁₂F₃N₅O₃ [M+H]⁺ 550.2, found 550.2.

Step d: To a solution of tert-butyl (R)-4-(4-((1-(2,4-dichlorophenyl)ethyl)amino)-5-(2,2,2-trifluoroethoxy)pyrimidin-2-yl)piperazine-1-carboxylate (0.53 g, 0.96 mmol) in DCM (1.6 mL) was added 4.0 M HCl in dioxane (2.4 mL, 9.6 mmol). The mixture was stirred at room temperature for 2 h and then concentrated to dryness to provide N-[(1R)-1-(2,4-dichlorophenyl)ethyl]-2-piperazin-1-yl-5-(2,2,2-trifluoroethoxy)pyrimidin-4-amine. MS: (ES) m/z calculated for C₁₈H₂₁C₁₂F₃N₅O [M+H]⁺ 450.1, found 450.1.

Step e: To a solution of N-[(1R)-1-(2,4-dichlorophenyl)ethyl]-2-piperazin-1-yl-5-(2,2,2-trifluoroethoxy)pyrimidin-4-amine (100 mg, 0.191 mmol) and (2R)-1-tert-butoxycarbonylpyrrolidine-2-carboxylic acid (49 mg, 0.23 mmol) in DMF (0.8 mL) was added HATU (94 mg, 0.25 mmol) and DIPEA (0.2 mL, 1.1 mmol). The contents were stirred at room temperature for 4 h. The reaction mixture was concentrated and then diluted with ethyl acetate and water. The aqueous layer was extracted with ethyl acetate. The combined organic layers were dried with sodium sulfate, filtered and concentrated. The crude material was purified on silica gel column chromatography to produce tert-butyl (R)-2-(4-(4-(((R)-1-(2,4-dichlorophenyl)ethyl)amino)-5-(2,2,2-trifluoroethoxy)pyrimidin-2-yl)piperazine-1-carbonyl)pyrrolidine-1-carboxylate. MS: (ES) m/z calculated for C₂₈H₃₆C₁₂F₃N₆O₄ [M+H]⁺ 647.2, found 647.3.

Step f: To a solution of tert-butyl (R)-2-(4-(4-(((R)-1-(2,4-dichlorophenyl)ethyl)amino)-5-(2,2,2-trifluoroethoxy)pyrimidin-2-yl)piperazine-1-carbonyl)pyrrolidine-1-carboxylate (100 mg, 0.154 mmol) in DCM (0.2 mL) was added 4.0 M HCl in dioxane (0.4 mL, 1.6 mmol). The mixture was stirred at room temperature for 2 h and then concentrated to dryness to provide 2-(4-(D-prolyl)piperazin-1-yl)-4-(((R)-1-(2,4-dichlorophenyl)ethyl)amino)-5-(2,2,2-trifluoroethoxy)pyrimidine. ¹H NMR (400 MHz, CD₃OD) δ 7.57 (s, 1H), 7.50 (s, 1H), 7.42 (d, J=8.8 Hz, 1H), 7.33 (d, J=8.0 Hz, 1H), 5.70-5.50 (m, 1H), 4.76-4.68 (m, 3H), 3.90-3.72 (m, 3H), 3.70-3.57 (m, 4H), 3.56-3.32 (m, 3H), 2.55-2.50 (m, 1H), 2.13-2.00 (m, 2H), 1.99-1.93 (m, 1H), 1.60 (d, J=6.8 Hz, 3H). MS: (ES) m/z calculated for C₂₃H₂₈C₁₂F₃N₆O₂ [M+H]⁺ 547.2, found 547.2.

### Example 38: Synthesis of (R)-5-(4-(5-chloro-4-(((R)-1-(2,4-dichlorophenyl)ethyl)amino)pyrimidin-2-yl)-1,2,3,6-tetrahydropyridine-1-carbonyl)pyrrolidin-2-one

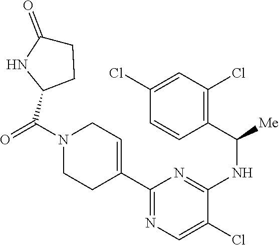 [SMILES: C[C@@H](Nc1nc(C2=CCN(C(=O)[C@H]3CCC(=O)N3)CC2)ncc1Cl)c1ccc(Cl)cc1Cl]

Step a: A mixture of (R)-2,5-dichloro-N-(1-(2,4-dichlorophenyl)ethyl)pyrimidin-4-amine (702 mg, 2.08 mmol), tert-butyl 4-(4,4,5,5-tetramethyl-1,3,2-dioxaborolan-2-yl)-3,6-dihydro-2H-pyridine-1-carboxylate (810 mg, 2.62 mmol), and potassium carbonate (1.40 g, 4.87 mmol) in a 10:3 solution of toluene/H₂O (13 mL) was sparged with N₂ gas for 5 min. Pd(dppf)Cl₂-DCM complex (120 mg, 0.0705 mmol) was added and the mixture was stirred at 120° C. for 2 h. The contents were diluted with H₂O and extracted with ethyl acetate. The organic layers were dried over MgSO₄, filtered, and the filtrate was concentrated. The crude residue was purified by silica gel column chromatography to yield tert-butyl (R)-4-(5-chloro-4-((1-(2,4-dichlorophenyl)ethyl)amino)pyrimidin-2-yl)-3,6-dihydropyridine-1(2H)-carboxylate. MS: (ES) m/z calculated for C₂₂H₂₆Cl₃N₄₀₂ [M+H]⁺ 483.1, found 483.1.

Step b: To a solution of tert-butyl (R)-4-(5-chloro-4-((1-(2,4-dichlorophenyl)ethyl)amino)pyrimidin-2-yl)-3,6-dihydropyridine-1(2H)-carboxylate (900 mg, 1.86 mmol) in DCM (4 mL) was added 4.0 M HCl in dioxane (3.0 mL, 12 mmol). The mixture was stirred at room temperature for 2 h and then concentrated to dryness to provide (R)-5-chloro-N-(1-(2,4-dichlorophenyl)ethyl)-2-(1,2,3,6-tetrahydropyridin-4-yl)pyrimidin-4-amine. MS: (ES) m/z calculated for C₁₇H₁₈Cl₃N₄ [M+H]⁺ 383.1, found 383.1.

Step c: To a solution of (R)-5-chloro-N-(1-(2,4-dichlorophenyl)ethyl)-2-(1,2,3,6-tetrahydropyridin-4-yl)pyrimidin-4-amine (200 mg, 0.438 mmol) and (2R)-5-oxopyrrolidine-2-carboxylic acid (68 mg, 0.53 mmol) in DMF (1.8 mL) was added HATU (250 mg, 0.657 mmol) and DIPEA (0.5 mL, 2.6 mmol). The contents were stirred at room temperature for 2 h. The reaction mixture was concentrated and then diluted with EtOAc and water. The aqueous layer was extracted with EtOAc. The combined organic layers were dried with sodium sulfate, filtered and concentrated. The crude material was purified on silica gel column chromatography to produce (R)-5-(4-(5-chloro-4-(((R)-1-(2,4-dichlorophenyl)ethyl)amino)pyrimidin-2-yl)-1,2,3,6-tetrahydropyridine-1-carbonyl)pyrrolidin-2-one. ¹H NMR (400 MHz, CD₃OD) δ 8.39 (s, 1H), 7.51 (s, 1H), 7.44 (d, J=8.8 Hz, 1H), 7.33 (d, J=8.0 Hz, 1H), 7.02 (s, 1H), 5.90-5.70 (m, 1H), 4.80-4.60 (m, 1H), 4.50-4.20 (m, 2H), 3.80-3.65 (m, 2H), 2.80-2.30 (m, 5H), 2.10-1.90 (m, 1H), 1.64 (d, J=6.8 Hz, 3H). MS: (ES) m/z calculated for C₂₂H₂₃Cl₃N₅O₂ [M+H]⁺ 494.1, found 494.1.

### Example 39: Synthesis of (R)-5-(4-(5-chloro-4-(((R)-1-(2,4-dichlorophenyl)ethyl)amino)pyrimidin-2-yl)piperazine-1-carbonyl)pyrrolidin-2-one

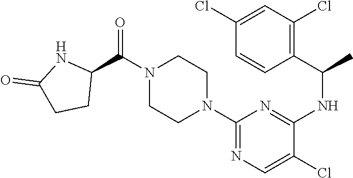 [SMILES: C[C@@H](Nc1nc(N2CCN(C(=O)[C@H]3CCC(=O)N3)CC2)ncc1Cl)c1ccc(Cl)cc1Cl]

To a solution of 5-chloro-N-[(1R)-1-(2,4-dichlorophenyl)ethyl]-2-piperazin-1-yl-pyrimidin-4-amine (50 mg, 0.11 mmol) in DMF (1 mL) was added (2R)-5-oxopyrrolidine-2-carboxylic acid (21 mg, 0.16 mmol), EDCI (31 mg, 0.16 mmol), and 4-(N,N-dimethylamino)pyridine (13 mg, 0.11 mmol). The reaction was stirred at 25° C. for 24 h and then quenched with water. The contents were extracted with ethyl acetate and the combined organic layers were dried with sodium sulfate, filtered and concentrated. The crude material was purified by silica gel column chromatography to give (R)-5-(4-(5-chloro-4-(((R)-1-(2,4-dichlorophenyl)ethyl)amino)pyrimidin-2-yl)piperazine-1-carbonyl)pyrrolidin-2-one. ¹H NMR (400 MHz, CD₃OD) δ 7.81 (s, 1H), 7.46 (s, 1H), 7.37 (dd, J=8.5, 1.9 Hz, 1H), 7.29-7.24 (m, 1H), 7.07 (d, J=6.2 Hz, 1H), 5.57-5.40 (m, 1H), 4.69 (dd, J=9.7, 4.5 Hz, 1H), 3.76-3.35 (m, 8H), 2.56-2.42 (m, 1H), 2.39-2.29 (m, 2H), 2.07-1.95 (m, 1H), 1.52 (dd, J=6.9, 2.0 Hz, 3H). MS: (ES) m/z calculated for C₂₁H₂₄Cl₃N₆O₂ [M+H]⁺ 497.1, found 497.1.

### Example 40: Synthesis of (R)-4-(4-(5-chloro-4-(((R)-1-(2,4-dichlorophenyl)ethyl)amino)pyrimidin-2-yl)piperazine-1-carbonyl)-1-methylimidazolidin-2-one

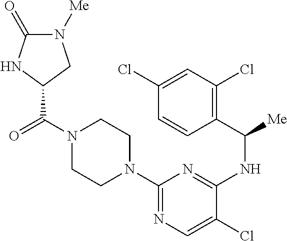 [SMILES: C[C@@H](Nc1nc(N2CCN(C(=O)[C@H]3CN(C)C(=O)N3)CC2)ncc1Cl)c1ccc(Cl)cc1Cl]

Step a: To a solution of benzyl (4R)-1-methyl-2-oxo-imidazolidine-4-carboxylate (250 mg, 1.1 mmol) in 4:1 THE/H₂O (5 mL) was added lithium hydroxide monohydrate (49 mg, 1.2 mmol). The reaction was stirred at 55° C. for 3 h and then concentrated. The residue was diluted with MeCN (5 mL) and the solid was washed with Et₂O and dried in vacuo to give lithium (4R)-1-methyl-2-oxo-imidazolidine-4-carboxylate. C₅H₇N₂₀₃ [M−H]⁻ 143.0, found 143.0.

Step b: To a solution of 5-chloro-N-[(1R)-1-(2,4-dichlorophenyl)ethyl]-2-piperazin-1-yl-pyrimidin-4-amine (205 mg, 0.11 mmol) in DMF (1 mL) was added lithium (4R)-1-methyl-2-oxo-imidazolidine-4-carboxylate (18 mg, 0.12 mmol), EDCI (26 mg, 0.16 mmol), and 4-(N,N-dimethylamino)pyridine (13 mg, 0.11 mmol). The reaction was stirred at 25° C. for 24 h and then quenched with water. The contents were extracted with EtOAc and the combined organic layers were dried with sodium sulfate, filtered and concentrated. The crude material was purified by silica gel column chromatography to give (R)-4-(4-(5-chloro-4-(((R)-1-(2,4-dichlorophenyl)ethyl)amino)pyrimidin-2-yl)piperazine-1-carbonyl)-1-methylimidazolidin-2-one.

¹H NMR (400 MHz, CD₃OD) δ 7.80 (s, 1H), 7.48-7.44 (m, 1H), 7.40-7.35 (m, 1H), 7.29-7.24 (m, 1H), 7.06 (d, J=6.0 Hz, 1H), 5.55-5.44 (m, 1H), 4.68-4.59 (m, 1H), 3.79-3.67 (m, 2H), 3.58 (q, J=8.0, 6.7 Hz, 4H), 3.52-3.38 (m, 4H), 2.80-2.70 (m, 3H), 1.52 (d, J=7.9 Hz, 3H). MS: (ES) m/z calculated for C₂₁H₂₅Cl₃N₇₀₂ [M+H]⁺ 512.1, found 512.1.

### Example 41: Synthesis of (R)-5-((S)-4-(5-chloro-4-(((R)-1-(2,4-dichlorophenyl)ethyl)amino)pyrimidin-2-yl)-3-methylpiperazine-1-carbonyl)pyrrolidin-2-one

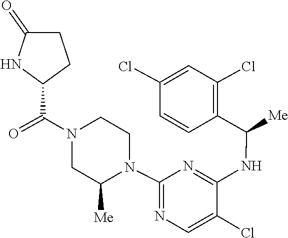 [SMILES: C[C@@H](Nc1nc(N2CCN(C(=O)[C@H]3CCC(=O)N3)C[C@@H]2C)ncc1Cl)c1ccc(Cl)cc1Cl]

Step a: To a solution of 2,5-dichloro-N-[(1R)-1-(2,4-dichlorophenyl)ethyl]pyrimidin-4-amine (100 mg, 0.28 mmol) in DMSO (1 mL) was added tert-butyl (3S)-3-methylpiperazine-1-carboxylate (57 mg, 0.28 mmol), and DIPEA (0.055 mL, 0.31 mmol). The reaction was stirred at 90° C. for 24 h and then quenched with water. The contents were extracted with ethyl acetate and the combined organic layers were dried with sodium sulfate, filtered and concentrated. The crude material was purified by silica gel column chromatography to give tert-butyl (S)-4-(5-chloro-4-(((R)-1-(2,4-dichlorophenyl)ethyl)amino)pyrimidin-2-yl)-3-methylpiperazine-1-carboxylate. MS: (ES) m/z calculated for C₂₂H₂₉Cl₃N₅O₂ [M+H]⁺ 500.1, found 500.1.

Step b: A solution of tert-butyl (S)-4-(5-chloro-4-(((R)-1-(2,4-dichlorophenyl)ethyl)amino)pyrimidin-2-yl)-3-methylpiperazine-1-carboxylate (50 mg, 0.090 mmol) in 4 N HCl in dioxane (2 mL) was stirred 30 min at 25° C. The mixture was then diluted with Et₂O to precipitate a solid. The suspension was filtered and the solid was dried in vacuo to give 5-chloro-N-((R)-1-(2,4-dichlorophenyl)ethyl)-2-((S)-2-methylpiperazin-1-yl)pyrimidin-4-amine. MS: (ES) m/z calculated for C₁₇H₂₁Cl₃N₅ [M+H]⁺ 400.1, found 400.1.

Step c: To a solution of 5-chloro-N-((R)-1-(2,4-dichlorophenyl)ethyl)-2-((S)-2-methylpiperazin-1-yl)pyrimidin-4-amine (44 mg, 0.10 mmol) in DCM (1 mL) was added (2R)-5-oxopyrrolidine-2-carboxylic acid (14 mg, 0.11 mmol), HATU (42 mg, 0.16 mmol), and DIPEA (0.052 mL, 0.30 mmol). The reaction was stirred at 25° C. for 30 min and then quenched with water. The contents were extracted with EtOAc and the combined organic layers were dried with sodium sulfate, filtered and concentrated. The crude material was purified by silica gel column chromatography to give (R)-5-((S)-4-(5-chloro-4-(((R)-1-(2,4-dichlorophenyl)ethyl)amino)pyrimidin-2-yl)-3-methylpiperazine-1-carbonyl)pyrrolidin-2-one.

¹H NMR (400 MHz, CD₃OD) δ 7.80 (s, 1H), 7.48-7.43 (m, 1H), 7.40-7.35 (m, 1H), 7.31-7.21 (m, 1H), 7.04-6.95 (m, 1H), 5.47 (q, J=7.0 Hz, 1H), 4.81-4.62 (m, 1H), 4.61-4.51 (m, 1H), 4.34-4.13 (m, 2H), 3.94-3.64 (m, 1H), 3.20-2.69 (m, 3H), 2.57-2.23 (m, 3H), 2.14-1.88 (m, 1H), 1.52 (d, J=7.0 Hz, 3H), 1.19-1.00 (m, 3H). MS: (ES) m/z calculated for C₂₂H₂₆Cl₃N₆O₂ [M+H]⁺ 511.1, found 511.1.

### Example 42: Synthesis of (2R,5S)-N-((3S,4S)-1-(5-chloro-4-(((R)-1-(2,4-dichlorophenyl)ethyl)amino)pyrimidin-2-yl)-3-fluoropiperidin-4-yl)-5-methylpyrrolidine-2-carboxamide

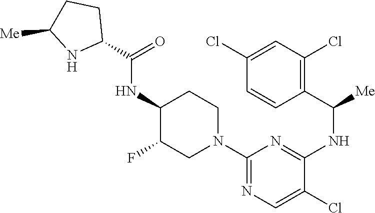 [SMILES: C[C@H]1CC[C@H](C(=O)N[C@H]2CCN(c3ncc(Cl)c(N[C@H](C)c4ccc(Cl)cc4Cl)n3)C[C@@H]2F)N1]

Step a: To a mixture of tert-butyl (3S,4S)-4-amino-3-fluoro-piperidine-1-carboxylate (250 mg, 1.1 mmol), isobenzofuran-1,3-dione (170 mg, 1.1 mmol), and zinc(II) bromide (260 mg, 1.1 mmol) in toluene (6 mL) was added trimethyl(trimethylsilyloxy)silane (280 mg, 1.7 mmol). The reaction was stirred at 120° C. for 18 h and then quenched with water. The contents were extracted with 9:1 methanol/ethyl acetate and the combined organic layers were dried with sodium sulfate, filtered and concentrated. The crude material was purified by silica gel column chromatography to give 2-[(3S,4S)-3-fluoro-4-piperidyl]isoindoline-1,3-dione. MS: (ES) m/z calculated for C₁₃H₁₄FN₂O₂ [M+H]⁺ 249.1, found 249.1.

Step b: To a solution of 2,5-dichloro-N-[(1R)-1-(2,4-dichlorophenyl)ethyl]pyrimidin-4-amine (102 mg, 0.30 mmol) in DMF (2 mL) was added 2-[(3S,4S)-3-fluoro-4-piperidyl]isoindoline-1,3-dione (75 mg, 0.30 mmol). The reaction was stirred at 90° C. for 24 h and then concentrated. The crude material was dissolved in methanol (1 mL) and hydrazine (0.095 mL, 3.0 mmol) was added. The resulting mixture was stirred for 2 h and then filtered.

The filtrate was concentrated and the crude material was purified by silica gel column chromatography to give 2-[(3S,4S)-4-amino-3-fluoro-1-piperidyl]-5-chloro-N-[(1R)-1-(2,4-dichlorophenyl)ethyl]pyrimidin-4-amine. MS: (ES) m/z calculated for C₁₇H₂₀Cl₃FN₅ [M+H]⁺ 418.1, found 418.1.

Step c: To a solution of 2-[(3S,4S)-4-amino-3-fluoro-1-piperidyl]-5-chloro-N-[(1R)-1-(2,4-dichlorophenyl)ethyl]pyrimidin-4-amine (23 mg, 0.050 mmol) in DCM (1 mL) was added (2R,5S)-1-(tert-butoxycarbonyl)-5-methylpyrrolidine-2-carboxylic acid (13 mg, 0.055 mmol), HATU (21 mg, 0.55 mmol), and DIEA (0.026 mL, 0.15 mmol). The reaction was stirred at 23° C. for 16 h and then quenched with water. The contents were extracted with EtOAc and the combined organic layers were dried with sodium sulfate, filtered and concentrated. The crude material was purified by silica gel column chromatography to give tert-butyl (2R,5S)-2-(((3S,4S)-1-(5-chloro-4-(((R)-1-(2,4-dichlorophenyl)ethyl)amino)pyrimidin-2-yl)-3-fluoropiperidin-4-yl)carbamoyl)-5-methylpyrrolidine-1-carboxylate. MS: (ES) m/z calculated for C₂₈H₃₇Cl₃FN₆O₃ [M+H]⁺ 629.2, found 629.2.

Step d: A solution of tert-butyl (2R,5S)-2-(((3S,4S)-1-(5-chloro-4-(((R)-1-(2,4-dichlorophenyl)ethyl)amino)pyrimidin-2-yl)-3-fluoropiperidin-4-yl)carbamoyl)-5-methylpyrrolidine-1-carboxylate (30 mg, 0.011 mmol) in 4 N HCl in 1,4-dioxane (1 mL) was stirred 30 min at 25° C. The mixture was then diluted with 20 mL Et₂O to precipitate a solid. The suspension was filtered and the solid was dried in vacuo to give (2R,5S)-N-((3S,4S)-1-(5-chloro-4-(((R)-1-(2,4-dichlorophenyl)ethyl)amino)pyrimidin-2-yl)-3-fluoropiperidin-4-yl)-5-methylpyrrolidine-2-carboxamide. ¹H NMR (400 MHz, DMSO-d₆) δ 9.08-8.93 (m, 1H), 8.79-8.66 (m, 1H), 8.51 (d, J=8.1 Hz, 1H), 7.85 (s, 1H), 7.49 (d, J=2.1 Hz, 1H), 7.40 (d, J=8.4 Hz, 1H), 7.32 (dd, J=8.4, 2.1 Hz, 1H), 5.34 (p, J=7.0 Hz, 1H), 4.46-3.86 (m, 7H), 3.10-2.89 (m, 2H), 2.35-2.24 (m, 1H), 2.10-1.98 (m, 1H), 1.80-1.58 (m, 2H), 1.57-1.44 (m, 1H), 1.40 (d, J=7.1 Hz, 3H), 1.24 (d, J=6.5 Hz, 3H). MS: (ES) m/z calculated for C₂₃H₂₉Cl₃FN₆O [M+H]⁺ 529.1, found 529.1.

### Example 43: Synthesis of (2R,5S)-N-((3S,4S)-1-(4-(((R)-1-(2,4-dichlorophenyl)ethyl)amino)-5-(difluoromethoxy)pyrimidin-2-yl)-3-hydroxypiperidin-4-yl)-5-methylpyrrolidine-2-carboxamide

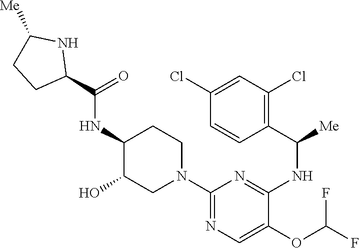 [SMILES: C[C@H]1CC[C@H](C(=O)N[C@H]2CCN(c3ncc(OC(F)F)c(N[C@H](C)c4ccc(Cl)cc4Cl)n3)C[C@@H]2O)N1]

Step a: A mixture of formic acid (2.3 mL, 60.0 mmol) and acetic anhydride (3.4 mL, 36.4 mmol) was stirred at 60° C. for 1 h. The reaction was cooled to 25° C. and then 2,4-dichloropyrimidin-5-ol (1.0 g, 6.1 mmol) and sodium acetate (250 mg, 3.0 mmol) were added. The reaction was stirred at 25° C. for 2 h and then quenched with saturated NaHCO₃ (aq). The biphasic mixture was shaken with toluene and the resulting layers separated. The organic layer was washed with H₂O and brine and then dried with sodium sulfate, filtered and concentrated. The crude material was purified by silica gel column chromatography to give (2,4-dichloropyrimidin-5-yl) formate. MS: (ES) m/z calculated for C₅H₃Cl₂N₂O₂ [M+H]⁺ 193.0, found 193.0.

Step b: To a solution of (2,4-dichloropyrimidin-5-yl) formate (460 mg, 2.4 mmol) in DCM (2.5 mL) was added N-ethyl-N-(trifluoro-κ4-sulfanyl)ethanamine (1.2 g, 7.2 mmol) dropwise at 25° C. The reaction was stirred 48 h at 25° C. and then quenched with saturated NaHCO₃ (aq). The biphasic mixture was extracted with DCM and the combined organic layers were dried with sodium sulfate, filtered and concentrated. The crude material was purified by silica gel column chromatography to give 2,4-dichloro-5-(difluoromethoxy)pyrimidine. MS: (ES) m/z calculated for C₅H₃C₁₂F₂N₂₀ [M+H]⁺ 215.0, found 215.0.

Step c: To a solution of 2,4-dichloro-5-(difluoromethoxy)pyrimidine (170 mg, 0.79 mmol) and DIPEA (0.55 mL, 3.1 mmol) in DMF (3 mL) was added (1R)-1-(2,4-dichlorophenyl)ethanamine (150 mg, 0.79 mmol). The reaction was stirred at 90° C. for 16 h and then quenched with water. The contents were extracted with EtOAc and the combined organic layers were dried with sodium sulfate, filtered and concentrated. The crude material was purified by silica gel column chromatography to give 2-chloro-N-[(1R)-1-(2,4-dichlorophenyl)ethyl]-5-(difluoromethoxy)pyrimidin-4-amine. MS: (ES) m/z calculated for C₁₃H₁₁Cl₃F₂N₃O [M+H]⁺ 368.0, found 368.0.

Step d: To a solution of 2-chloro-N-[(1R)-1-(2,4-dichlorophenyl)ethyl]-5-(difluoromethoxy)pyrimidin-4-amine (200 mmol, 0.54 mmol) and DIPEA (0.19 mL, 1.1 mmol) in DMF (1.1 mL) was added tert-butyl N-[(3S,4S)-3-hydroxy-4-piperidyl]carbamate (120 mg, 0.55 mmol). The reaction was stirred at 90° C. for 1 h and then quenched with water. The contents were extracted with EtOAc and the combined organic layers were dried with sodium sulfate, filtered and concentrated. The crude material was purified by silica gel column chromatography to give tert-butyl ((3S,4S)-1-(4-(((R)-1-(2,4-dichlorophenyl)ethyl)amino)-5-(difluoromethoxy)pyrimidin-2-yl)-3-hydroxypiperidin-4-yl)carbamate. MS: (ES) m/z calculated for C₂₃H₃₀Cl₂F₂N₅O₄ [M+H]⁺ 548.2, found 548.2.

Step e: A solution of tert-butyl ((3S,4S)-1-(4-(((R)-1-(2,4-dichlorophenyl)ethyl)amino)-5-(difluoromethoxy)pyrimidin-2-yl)-3-hydroxypiperidin-4-yl)carbamate (85 mg, 0.15 mmol) in 4 N HCl in dioxane was stirred for 30 min at 25° C. The mixture was then diluted with Et₂O to precipitate a solid. The suspension was filtered and the solid was dried in vacuo to give (3S,4S)-4-amino-1-[4-[[(1R)-1-(2,4-dichlorophenyl)ethyl]amino]-5-(difluoromethoxy)pyrimidin-2-yl]piperidin-3-ol. MS: (ES) m/z calculated for C₁₈H₂₂C₁₂F₂N₅O₂ [M+H]⁺ 448.1, found 448.1.

Step f: To a solution of (3S,4S)-4-amino-1-[4-[[(1R)-1-(2,4-dichlorophenyl)ethyl]amino]-5-(difluoromethoxy)pyrimidin-2-yl]piperidin-3-ol (40 mg, 0.077 mmol) in DCM (1 mL) was added (2R,5S)-5-methylpyrrolidine-2-carboxylic acid (19 mg, 0.084 mmol), HATU (32 mg, 0.084 mmol), and DIPEA (0.051 mL, 0.31 mmol). The reaction was stirred at 23° C. for 16 h and then quenched with water. The contents were extracted with EtOAc and the combined organic layers were dried with sodium sulfate, filtered and concentrated. The crude material was purified by silica gel column chromatography to give tert-butyl (2R,5S)-2-(((3S,4S)-1-(4-(((R)-1-(2,4-dichlorophenyl)ethyl)amino)-5-(difluoromethoxy)pyrimidin-2-yl)-3-hydroxypiperidin-4-yl)carbamoyl)-5-methylpyrrolidine-1-carboxylate. MS: (ES) m/z calculated for C₂₉H₃₉C₁₂F₂N₆O₅ [M+H]⁺ 659.2, found 659.2.

Step g: A solution of tert-butyl (2R,5S)-2-(((3S,4S)-1-(4-(((R)-1-(2,4-dichlorophenyl)ethyl)amino)-5-(difluoromethoxy)pyrimidin-2-yl)-3-hydroxypiperidin-4-yl)carbamoyl)-5-methylpyrrolidine-1-carboxylate (24 mg, 0.033 mmol) in 4 N HCl in 1,4-dioxane (1 mL) was stirred 30 min at 25° C. The mixture was then diluted with Et₂O to precipitate a solid. The suspension was filtered and the solid was dried in vacuo to give (2R,5S)-N-((3S,4S)-1-(4-(((R)-1-(2,4-dichlorophenyl)ethyl)amino)-5-(difluoromethoxy)pyrimidin-2-yl)-3-hydroxypiperidin-4-yl)-5-methylpyrrolidine-2-carboxamide. ¹H NMR (400 MHz, DMSO-d₆) δ 8.83-8.68 (m, 1H), 8.51-8.28 (m, 1H), 7.86-7.71 (m, 1H), 7.57 (s, 1H), 7.50-7.35 (m, 2H), 7.29-6.79 (m, 1H), 5.43 (d, J=10.8 Hz, 1H), 4.69-3.81 (m, 8H), 3.74-3.59 (m, 2H), 3.38-3.18 (m, 1H), 2.43-2.30 (m, 1H), 2.17-2.03 (m, 1H), 1.87-1.63 (m, 2H), 1.63-1.51 (m, 1H), 1.46 (d, J=6.7 Hz, 3H), 1.32 (d, J=4.5 Hz, 3H). MS: (ES) m/z calculated for C₂₄H₃₁C₁₂F₂N₆O₃. [M+H]⁺ 559.2, found 559.2.

### Example 44: Synthesis of 2-(4-(D-prolyl)piperazin-1-yl)-4-(((R)-1-(2,4-dichlorophenyl)ethyl)amino)-5-(difluoromethoxy)pyrimidine

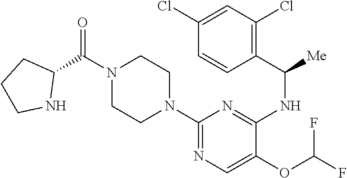 [SMILES: C[C@@H](Nc1nc(N2CCN(C(=O)[C@H]3CCCN3)CC2)ncc1OC(F)F)c1ccc(Cl)cc1Cl]

Step a: A mixture of 2-chloro-N-[(1R)-1-(2,4-dichlorophenyl)ethyl]-5-(difluoromethoxy)pyrimidin-4-amine (170 mmol, 0.45 mmol) and tert-butyl piperazine-1-carboxylate (1.3 g, 7.2 mmol). The reaction was stirred at 125° C. for 7 h and then cooled to 25° C. The crude material was purified by silica gel column chromatography to give tert-butyl (R)-4-(4-((1-(2,4-dichlorophenyl)ethyl)amino)-5-(difluoromethoxy)pyrimidin-2-yl)piperazine-1-carboxylate. MS: (ES) m/z calculated for C₂₂H₂₈C₁₂F₂N₅O₃ [M+H]⁺ 518.2, found 518.2.

Step b: A solution of tert-butyl (R)-4-(4-((1-(2,4-dichlorophenyl)ethyl)amino)-5-(difluoromethoxy)pyrimidin-2-yl)piperazine-1-carboxylate (130 mg, 0.25 mmol) in 4 N HCl in dioxane was stirred for 30 min at 25° C. The mixture was then diluted with Et₂O to precipitate a solid. The suspension was filtered and the solid was dried in vacuo to give N-[(1R)-1-(2,4-dichlorophenyl)ethyl]-5-(difluoromethoxy)-2-piperazin-1-yl-pyrimidin-4-amine. MS: (ES) m/z calculated for C₁₇H₂₀Cl₂F₂N₅O [M+H]⁺ 418.1, found 418.1.

Step c: To a solution of N-[(1R)-1-(2,4-dichlorophenyl)ethyl]-5-(difluoromethoxy)-2-piperazin-1-yl-pyrimidin-4-amine (40 mg, 0.081 mmol) in DCM (1 mL) was added (2R)-1-tert-butoxycarbonylpyrrolidine-2-carboxylic acid (19 mg, 0.090 mmol), HATU (34 mg, 0.090 mmol), and DIPEA (0.055 mL, 0.33 mmol). The reaction was stirred at 25° C. for 30 min and then quenched with water. The contents were extracted with EtOAc and the combined organic layers were dried with sodium sulfate, filtered and concentrated. The crude material was purified by silica gel column chromatography to give tert-butyl (R)-2-(4-(4-(((R)-1-(2,4-dichlorophenyl)ethyl)amino)-5-(difluoromethoxy)pyrimidin-2-yl)piperazine-1-carbonyl)pyrrolidine-1-carboxylate. MS: (ES) m/z calculated for C₂₇H₃₅C₁₂F₂N₆O₄ [M+H]⁺ 615.2, found 615.2.

Step g: A solution of tert-butyl (R)-2-(4-(4-(((R)-1-(2,4-dichlorophenyl)ethyl)amino)-5-(difluoromethoxy)pyrimidin-2-yl)piperazine-1-carbonyl)pyrrolidine-1-carboxylate (40 mg, 0.061 mmol) in 4 N HCl in 1,4-dioxane (1 mL) was stirred 30 min at 25° C. The mixture was then diluted with Et₂O to precipitate a solid. The suspension was filtered and the solid was dried in vacuo to give 2-(4-(D-prolyl)piperazin-1-yl)-4-(((R)-1-(2,4-dichlorophenyl)ethyl)amino)-5-(difluoromethoxy)pyrimidine. ¹H NMR (400 MHz, CD₃OD) δ 9.34 (d, J=6.5 Hz, 1H), 7.76 (s, 1H), 7.51 (s, 1H), 7.41-7.35 (m, 1H), 7.35-7.28 (m, 1H), 6.89 (t, J=72.2, 70.5 Hz, 1H), 5.71-5.58 (m, 1H), 4.73 (t, J=7.5 Hz, 1H), 3.96-3.35 (m, 11H), 2.59-2.45 (m, 1H), 2.19-1.88 (m, 3H), 1.59 (d, J=7.2 Hz, 3H). MS: (ES) m/z calculated for C₂₂H₂₇C₁₂F₂N₆O₂. [M+H]⁺ 515.2, found 515.2.

### Example 45: Synthesis of 5-chloro-N-((R)-1-(2,4-dichlorophenyl)ethyl)-2-(4-(((S)-pyrrolidin-3-yl)oxy)piperidin-1-yl)pyrimidin-4-amine

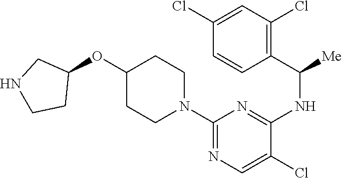 [SMILES: C[C@@H](Nc1nc(N2CCC(O[C@H]3CCNC3)CC2)ncc1Cl)c1ccc(Cl)cc1Cl]

Step a: To a mixture of pyridin-4-ol (500 mg, 5.3 mmol), tert-butyl (3R)-3-hydroxypyrrolidine-1-carboxylate (980 mg, 5.3 mmol), triphenylphosphine (1.4 g, 5.3 mmol) in THE (18 mL) was added DIAD (1.1 g, 5.3 mmol). The reaction was stirred at 55° C. for 24 h and then concentrated. The crude material was purified by silica gel column chromatography to give tert-butyl (3R)-3-(4-pyridyloxy)pyrrolidine-1-carboxylate. MS: (ES) m/z calculated for C₁₄H₂₁N₂O₃ [M+H]⁺ 265.2, found 265.2.

Step b: To a solution of tert-butyl (3R)-3-(4-pyridyloxy)pyrrolidine-1-carboxylate (580 mg, 2.2 mmol) in MeOH (2 mL) was added platinum(IV) oxide (50 mg, 0.22 mmol), and conc HCl (0.36 mL, 4.4 mmol). The reaction was shaken in a Parr reactor under H₂ (50 psi) at 20° C. for 24 h. The mixture was filtered through Celite and the filtrate was concentrated to give tert-butyl (3S)-3-(4-piperidyloxy)pyrrolidine-1-carboxylate. MS: (ES) m/z calculated for C₁₄H₂₇N₂O₃ [M+H]⁺ 271.2, found 271.2.

Step c: To a solution of 2,5-dichloro-N-[(1R)-1-(2,4-dichlorophenyl)ethyl]pyrimidin-4-amine (200 mg, 0.59 mmol) in DMF (2 mL) was added tert-butyl (3S)-3-(4-piperidyloxy)pyrrolidine-1-carboxylate hydrochloride (180 mg, 0.59 mmol), and DIPEA (0.21 mL, 1.2 mmol). The reaction was stirred at 80° C. for 24 h and then quenched with water. The contents were extracted with EtOAc and the combined organic layers were dried with sodium sulfate, filtered and concentrated. The crude material was purified by silica gel column chromatography to give tert-butyl (S)-3-((1-(5-chloro-4-(((R)-1-(2,4-dichlorophenyl)ethyl)amino)pyrimidin-2-yl)piperidin-4-yl)oxy)pyrrolidine-1-carboxylate. MS: (ES) m/z calculated for C₂₆H₃₅Cl₃N₅O₃ [M+H]⁺ 570.2, found 570.2.

Step d: A solution of tert-butyl (S)-3-((1-(5-chloro-4-(((R)-1-(2,4-dichlorophenyl)ethyl)amino)pyrimidin-2-yl)piperidin-4-yl)oxy)pyrrolidine-1-carboxylate (30 mg, 0.053 mmol) in 4 N HCl in 1,4-dioxane (2 mL) was stirred 30 min at 25° C. The mixture was then diluted with Et₂O to precipitate a solid. The suspension was filtered and the solid was dried in vacuo to give 5-chloro-N-((R)-1-(2,4-dichlorophenyl)ethyl)-2-(4-(((S)-pyrrolidin-3-yl)oxy)piperidin-1-yl)pyrimidin-4-amine. ¹H NMR (400 MHz, CDCl₃) δ 8.80 (d, J=4.0 Hz, 1H), 7.91 (s, 1H), 7.48 (s, 1H), 7.41-7.36 (m, 1H), 7.36-7.30 (m, 1H), 5.66-5.52 (m, 1H), 4.51-4.42 (m, 1H), 3.94-3.69 (m, 4H), 3.63-3.43 (m, 2H), 3.42-3.32 (m, 3H), 2.27-2.01 (m, 2H), 1.96-1.75 (m, 2H), 1.70-1.40 (m, 5H), 1.35-1.19 (m, 1H). MS: (ES) m/z calculated for C₂₁H₂₇Cl₃N₅O [M+H]⁺ 470.1, found 470.1.

### Example 46: Synthesis of 2-((S)-3-((1-(5-chloro-4-(((R)-1-(2,4-dichlorophenyl)ethyl)amino)pyrimidin-2-yl)piperidin-4-yl)oxy)pyrrolidin-1-yl)ethan-1-ol

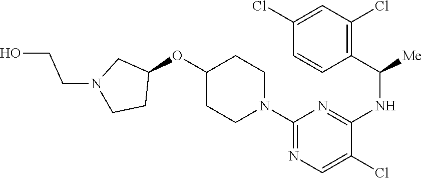 [SMILES: C[C@@H](Nc1nc(N2CCC(O[C@H]3CCN(CCO)C3)CC2)ncc1Cl)c1ccc(Cl)cc1Cl]

To a solution of 5-chloro-N-((R)-1-(2,4-dichlorophenyl)ethyl)-2-(4-(((S)-pyrrolidin-3-yl)oxy)piperidin-1-yl)pyrimidin-4-amine (16 mg, 0.030 mmol) in MeCN (1 mL) was added 2-iodoethanol (5.2 mg, 0.030 mmol) and potassium carbonate (24 mg, 1.1 mmol). The reaction was stirred at 80° C. for 18 h and then concentrated. The crude material was purified by preparative HPLC to give 2-((S)-3-((1-(5-chloro-4-(((R)-1-(2,4-dichlorophenyl)ethyl)amino)pyrimidin-2-yl)piperidin-4-yl)oxy)pyrrolidin-1-yl)ethan-1-ol. ¹H NMR (400 MHz, CD₃OD) δ 7.77-7.73 (m, 1H), 7.43-7.40 (m, 1H), 7.38-7.33 (m, 1H), 7.29-7.23 (m, 1H), 6.97 (d, J=5.2 Hz, 1H), 5.49-5.39 (m, 1H), 4.51-4.41 (m, 1H), 4.08-3.97 (m, 2H), 3.86-3.80 (m, 2H), 3.67-3.57 (m, 2H), 3.43-3.34 (m, 4H), 3.21-3.07 (m, 3H), 2.22-2.10 (m, 1H), 1.78-1.65 (m, 2H), 1.51 (d, J=8.1 Hz, 3H), 1.37-1.26 (m, 1H), 1.20-1.06 (m, 1H). MS: (ES) m/z calculated for C₂₃H₃₁Cl₃N₅O₂ [M+H]⁺ 514.2, found 514.2.

### Example 47: Synthesis of 5-chloro-N-((R)-1-(2,4-dichlorophenyl)ethyl)-2-(4-(((S)-1-methylpyrrolidin-3-yl)oxy)piperidin-1-yl)pyrimidin-4-amine

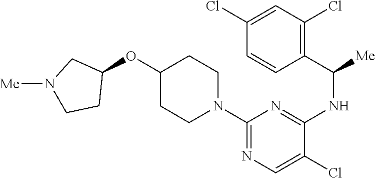 [SMILES: C[C@@H](Nc1nc(N2CCC(O[C@H]3CCN(C)C3)CC2)ncc1Cl)c1ccc(Cl)cc1Cl]

To a solution of 5-chloro-N-((R)-1-(2,4-dichlorophenyl)ethyl)-2-(4-(((S)-pyrrolidin-3-yl)oxy)piperidin-1-yl)pyrimidin-4-amine (50 mg, 0.092 mmol) in MeCN (1 mL) was added aqueous formaldehyde solution (2.9 mg, 0.97 mmol), Et₃N (0.013 mL, 0.092 mmol), and sodium triacetoxyborohydride (24 mg, 1.1 mmol). The reaction was stirred at 55° C. for 24 h and then concentrated. The crude material was purified by preparative HPLC to give 5-chloro-N-((R)-1-(2,4-dichlorophenyl)ethyl)-2-(4-(((S)-1-methylpyrrolidin-3-yl)oxy)piperidin-1-yl)pyrimidin-4-amine. ¹H NMR (400 MHz, CD₃OD) δ 7.87 (s, 1H), 7.46 (s, 1H), 7.40-7.35 (m, 1H), 7.33-7.28 (m, 1H), 5.61-5.51 (m, 1H), 4.54-4.42 (m, 1H), 3.98-3.82 (m, 2H), 3.81-3.62 (m, 3H), 3.50-3.34 (m, 3H), 3.14 (d, J=12.5 Hz, 2H), 2.97 (s, 3H), 2.50-2.04 (m, 2H), 1.90-1.70 (m, 2H), 1.63-1.46 (m, 4H), 1.39 (s, 1H). MS: (ES) m/z calculated for C₂₂H₂₉Cl₃N₅O [M+H]⁺ 484.1, found 484.1.

### Example 48: Synthesis of 2-(1-(D-prolyl)piperidin-4-yl)-5-chloro-4-(((R)-1-(2,4-dichlorophenyl)ethyl)amino)pyrimidine

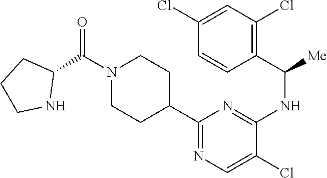 [SMILES: C[C@@H](Nc1nc(C2CCN(C(=O)[C@H]3CCCN3)CC2)ncc1Cl)c1ccc(Cl)cc1Cl]

Step a: To a solution of tert-butyl 4-carbamimidoylpiperidine-1-carboxylate (3.5 g, 15 mmol) in EtOH (70 mL) was added ethyl 2-chloro-3-oxo-propanoate (2.3 g, 15 mmol) and potassium carbonate (6.3 g, 45 mmol). The reaction was stirred at 80° C. for 16 h and then filtered through Celite. The filtrate was concentrated and the crude material was purified by silica gel column chromatography to give tert-butyl 4-(5-chloro-4-hydroxy-pyrimidin-2-yl)piperidine-1-carboxylate. MS: (ES) m/z calculated for C₁₄H₁₉C₁N₃O₃ [M−H]⁻ 312.1, found 312.1.

Step b: To a suspension of tert-butyl 4-(5-chloro-4-hydroxy-pyrimidin-2-yl)piperidine-1-carboxylate (1.0 g, 3.3 mmol) in toluene (16 mL) was added DIPEA (1.1 mL, 6.5 mmol) and then phosphorus(V) oxychloride (0.30 mL, 3.3 mmol) dropwise. The reaction was stirred at 120° C. for 1 h and then concentrated. The crude material was purified by silica gel column chromatography to give tert-butyl 4-(4,5-dichloropyrimidin-2-yl)piperidine-1-carboxylate. MS: (ES) m/z calculated for C₁₄H₂₀Cl₂N₃O₂ [M+H]⁺ 332.1, found 332.1.

Step c: To a solution of tert-butyl 4-(4,5-dichloropyrimidin-2-yl)piperidine-1-carboxylate (480 mg, 1.4 mmol) in DMF (5 mL) was added (1R)-1-(2,4-dichlorophenyl)ethanamine (270 mg, 1.4 mmol) and DIPEA (0.50 mL, 2.9 mmol). The reaction was stirred at 90° C. for 1 h and then quenched with water. The contents were extracted with EtOAc and the combined organic layers were dried with sodium sulfate, filtered and concentrated. The crude material was purified by silica gel column chromatography to give tert-butyl 4-[5-chloro-4-[[(1R)-1-(2,4-dichlorophenyl)ethyl]amino]pyrimidin-2-yl]piperidine-1-carboxylate. MS: (ES) m/z calculated for C₂₂H₂₈Cl₃N₄O₂ [M+H]⁺ 485.1, found 485.1.

Step d: A solution of tert-butyl 4-[5-chloro-4-[[(1R)-1-(2,4-dichlorophenyl)ethyl]amino]pyrimidin-2-yl]piperidine-1-carboxylate (400 mg, 0.82 mmol) in 4 N HCl in 1,4-dioxane (2 mL) was stirred 30 min at 25° C. The mixture was then diluted with Et₂O to precipitate a solid. The suspension was filtered and the solid was dried in vacuo to give 5-chloro-N-[(1R)-1-(2,4-dichlorophenyl)ethyl]-2-(4-piperidyl)pyrimidin-4-amine. MS: (ES) m/z calculated for C₁₇H₂₀Cl₃N₄ [M+H]⁺ 385.1, found 385.1.

Step e: To a solution of 5-chloro-N-[(1R)-1-(2,4-dichlorophenyl)ethyl]-2-(4-piperidyl)pyrimidin-4-amine (50 mg, 0.11 mmol) in DMF (1 mL) was added (2R)-1-tert-butoxycarbonylpyrrolidine-2-carboxylic acid (26 mg, 0.12 mmol), HATU (46 mg, 0.12 mmol), and DIPEA (0.50 mL, 2.9 mmol). The reaction was stirred at 25° C. for 18 h and then quenched with water. The contents were extracted with ethyl acetate and the combined organic layers were dried with sodium sulfate, filtered and concentrated. The crude material was purified by silica gel column chromatography to give tert-butyl (R)-2-(4-(5-chloro-4-(((R)-1-(2,4-dichlorophenyl)ethyl)amino)pyrimidin-2-yl)piperidine-1-carbonyl)pyrrolidine-1-carboxylate. MS: (ES) m/z calculated for C₂₇H₃₅Cl₃N₅₀₃ [M+H]⁺ 582.2, found 582.2.

Step f: A solution of tert-butyl (R)-2-(4-(5-chloro-4-(((R)-1-(2,4-dichlorophenyl)ethyl)amino)pyrimidin-2-yl)piperidine-1-carbonyl)pyrrolidine-1-carboxylate (30 mg, 0.053 mmol) in 4 N HCl in 1,4-dioxane (2 mL) was stirred 30 min at 25° C. The mixture was then diluted with Et₂O to precipitate a solid. The suspension was filtered and the solid was dried in vacuo to give 2-(1-(D-prolyl)piperidin-4-yl)-5-chloro-4-(((R)-1-(2,4-dichlorophenyl)ethyl)amino)pyrimidine. ¹H NMR (400 MHz, CD₃OD) δ 9.38 (s, 1H), 8.53-8.44 (m, 1H), 7.55-7.47 (m, 1H), 7.44-7.37 (m, 1H), 7.37-7.29 (m, 1H), 5.80-5.64 (m, 1H), 4.72 (dt, J=49.3, 8.3 Hz, 1H), 4.50 (dd, J=38.4, 13.3 Hz, 1H), 4.05-3.82 (m, 1H), 3.78-3.55 (m, 2H), 3.42 (dt, J=30.8, 9.0 Hz, 2H), 3.11-2.97 (m, 1H), 2.97-2.83 (m, 1H), 2.61-2.47 (m, 1H), 2.26-1.86 (m, 4H), 1.86-1.68 (m, 1H), 1.68-1.59 (m, 3H), 1.53-1.25 (m, 2H). MS: (ES) m/z calculated for C₂₂H₂₇Cl₃N₅O [M+H]⁺ 482.1, found 482.1.

### BIOLOGICAL EXAMPLES

### CEM Cells Endogenous Express CCR4

A chemotaxis assay can be used to determine the efficacy of potential receptor antagonists at blocking the migration mediated through chemokine receptors, such as CCR4. This assay is routinely performed using the ChemoTX® microchamber system with a 5 μm pore-sized polycarbonate membrane.

HBSS: CEM Cells are collected by centrifugation at 400×g at room temperature, then suspended at 5 million/ml in Hanks' Balanced Salt Solution (HBSS) buffer with 0.1% BSA. The compound being tested is serially diluted from a maximum final concentration of 10 μM (or an equivalent volume of its solvent (DMSO)) and is then added to the cell/buffer mixture. Separately, recombinant human CCL22 (MDC) at its EC₅₀ concentration (0.1 nM) is placed in the lower wells of the ChemoTX® plate. The 5-μm (pore size) polycarbonate membrane was placed onto the plate, and 20 μL of the cell/compound mixture is transferred onto each well of the membrane. The plates are incubated at 37° C. for 60 minutes, after which the polycarbonate membranes are removed and 5 μl of the DNA-intercalating dye CyQUANT (Invitrogen, Carlsbad, CA) is added to the lower wells. The amount of fluorescence, corresponding to the number of migrated cells, is measured using a Spectrafluor Plus plate reader (TECAN, San Jose, CA).

FBS: CEM Cells are collected by centrifugation at 400×g at room temperature, then suspended at 5 million/ml in 100% Fetal Bovine Serum (FBS) with 1% HEPEs. The compound being tested is serially diluted from a maximum final concentration of 10 μM (or an equivalent volume of its solvent (DMSO)) and is then added to the cell/buffer mixture. Separately, recombinant human CCL22 (MDC) at its EC₅₀ concentration (0.1 nM) is placed in the lower wells of the ChemoTX® plate. The 5-μm (pore size) polycarbonate membrane was placed onto the plate, and 20 μL of the cell/compound mixture is transferred onto each well of the membrane. The plates are incubated at 37° C. for 60 minutes, after which the polycarbonate membranes are removed and 5 μl of the DNA-intercalating dye CyQUANT (Invitrogen, Carlsbad, CA) is added to the lower wells. The amount of fluorescence, corresponding to the number of migrated cells, is measured using a Spectrafluor Plus plate reader (TECAN, San Jose, CA).

In the table below, activity is provided as follows:
IC50<100 nM (+++);
   >100 nM and <1000 nM (++); and
   >1000 nM.

TABLE 1

| | | Migration | Serum | |
|---|---|---|---|---|
| Compound | | IC₅₀ | IC₅₀ | MS |
| ID | Structure | (HBSS) | (FBS) | (M + H)+ |
| 1.001 | 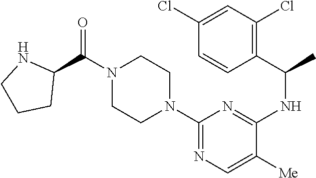 [SMILES: Cc1cnc(N2CCN(C(=O)[C@H]3CCCN3)CC2)nc1N[C@H](C)c1ccc(Cl)cc1Cl] | +++ | +++ | 463.2 |
| 1.002 | 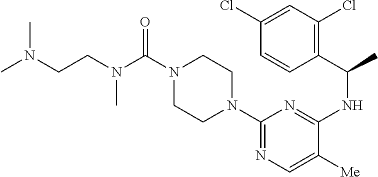 [SMILES: Cc1cnc(N2CCN(C(=O)N(C)CCN(C)C)CC2)nc1N[C@H](C)c1ccc(Cl)cc1Cl] | +++ | +++ | 514.2 |
| 1.003 | 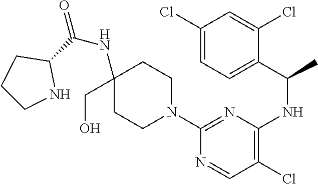 [SMILES: C[C@@H](Nc1nc(N2CCC(CO)(NC(=O)[C@H]3CCCN3)CC2)ncc1Cl)c1ccc(Cl)cc1Cl] | +++ | +++ | 527.2 |
| 1.004 | 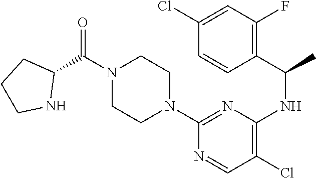 [SMILES: C[C@@H](Nc1nc(N2CCN(C(=O)[C@H]3CCCN3)CC2)ncc1Cl)c1ccc(Cl)cc1F] | +++ | +++ | 467.1 |
| 1.005 | 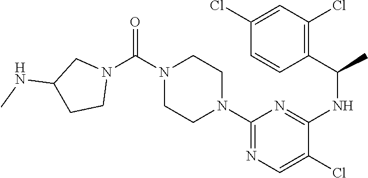 [SMILES: CNC1CCN(C(=O)N2CCN(c3ncc(Cl)c(N[C@H](C)c4ccc(Cl)cc4Cl)n3)CC2)C1] | +++ | +++ | 512.2 |
| 1.006 | 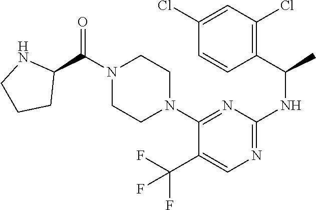 [SMILES: C[C@@H](Nc1ncc(C(F)(F)F)c(N2CCN(C(=O)[C@H]3CCCN3)CC2)n1)c1ccc(Cl)cc1Cl] | +++ | +++ | 517.2 |
| 1.007 | 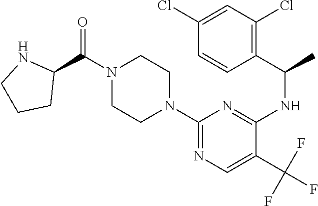 [SMILES: C[C@@H](Nc1nc(N2CCN(C(=O)[C@H]3CCCN3)CC2)ncc1C(F)(F)F)c1ccc(Cl)cc1Cl] | +++ | +++ | 517.2 |
| 1.008 | 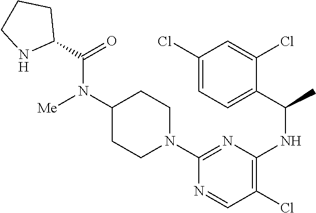 [SMILES: C[C@@H](Nc1nc(N2CCC(N(C)C(=O)[C@H]3CCCN3)CC2)ncc1Cl)c1ccc(Cl)cc1Cl] | +++ | +++ | 511.2 |
| 1.009 | 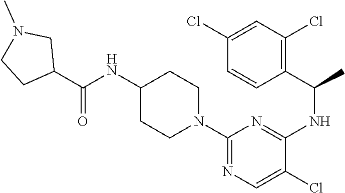 [SMILES: C[C@@H](Nc1nc(N2CCC(NC(=O)C3CCN(C)C3)CC2)ncc1Cl)c1ccc(Cl)cc1Cl] | +++ | +++ | 511.2 |
| 1.010 | 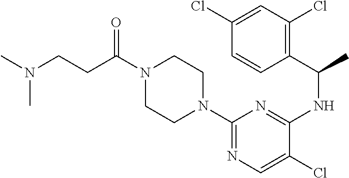 [SMILES: C[C@@H](Nc1nc(N2CCN(C(=O)CCN(C)C)CC2)ncc1Cl)c1ccc(Cl)cc1Cl] | +++ | +++ | 485.1 |
| 1.011 | 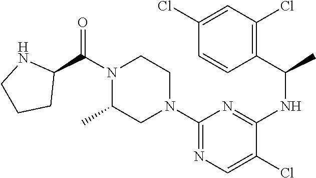 [SMILES: C[C@@H](Nc1nc(N2CCN(C(=O)[C@H]3CCCN3)[C@@H](C)C2)ncc1Cl)c1ccc(Cl)cc1Cl] | +++ | +++ | 497.1 |
| 1.012 | 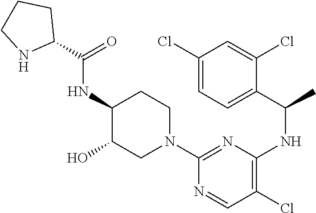 [SMILES: C[C@@H](Nc1nc(N2CC[C@H](NC(=O)[C@H]3CCCN3)[C@@H](O)C2)ncc1Cl)c1ccc(Cl)cc1Cl] | +++ | +++ | 513.2 |
| 1.013 | 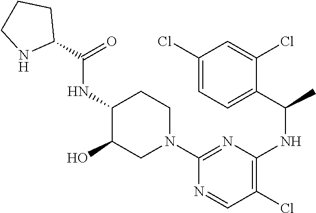 [SMILES: C[C@@H](Nc1nc(N2CC[C@@H](NC(=O)[C@H]3CCCN3)[C@H](O)C2)ncc1Cl)c1ccc(Cl)cc1Cl] | +++ | +++ | 513.2 |
| 1.014 | 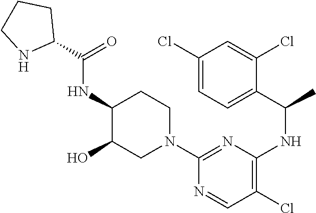 [SMILES: C[C@@H](Nc1nc(N2CC[C@H](NC(=O)[C@H]3CCCN3)[C@H](O)C2)ncc1Cl)c1ccc(Cl)cc1Cl] | +++ | +++ | 513.2 |
| 1.015 | 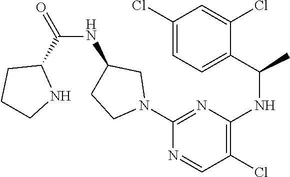 [SMILES: C[C@@H](Nc1nc(N2CC[C@@H](NC(=O)[C@H]3CCCN3)C2)ncc1Cl)c1ccc(Cl)cc1Cl] | +++ | +++ | 483.1 |
| 1.016 | 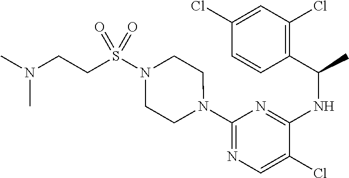 [SMILES: C[C@@H](Nc1nc(N2CCN(S(=O)(=O)CCN(C)C)CC2)ncc1Cl)c1ccc(Cl)cc1Cl] | +++ | ++ | 521.1 |
| 1.017 | 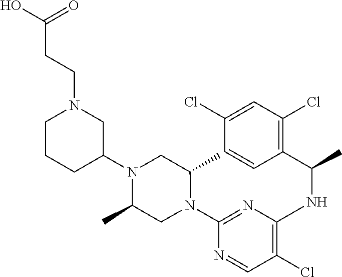 [SMILES: C[C@@H]1CN(c2ncc(Cl)c(N[C@H](C)c3ccc(Cl)cc3Cl)n2)[C@@H](C)CN1C1CCCN(CCC(=O)O)C1] | +++ | +++ | 569.2 |
| 1.018 | 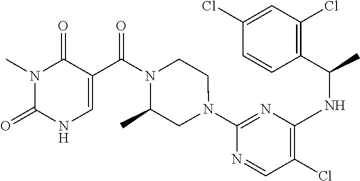 [SMILES: C[C@@H]1CN(c2ncc(Cl)c(N[C@H](C)c3ccc(Cl)cc3Cl)n2)CCN1C(=O)c1c[nH]c(=O)n(C)c1=O] | + | + | 552.1 |
| 1.019 | 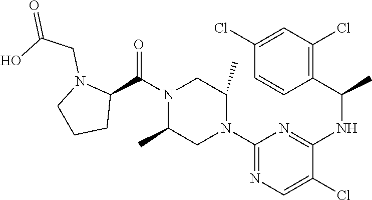 [SMILES: C[C@@H]1CN(c2ncc(Cl)c(N[C@H](C)c3ccc(Cl)cc3Cl)n2)[C@@H](C)CN1C(=O)[C@H]1CCCN1CC(=O)O] | ++ | ++ | 569.2 |
| 1.020 | 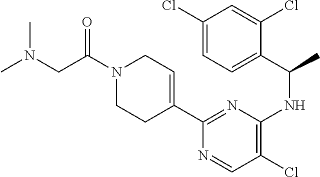 [SMILES: C[C@@H](Nc1nc(C2=CCN(C(=O)CN(C)C)CC2)ncc1Cl)c1ccc(Cl)cc1Cl] | +++ | +++ | 468.1 |
| 1.021 | 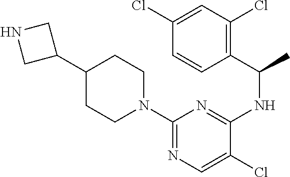 [SMILES: C[C@@H](Nc1nc(N2CCC(C3CNC3)CC2)ncc1Cl)c1ccc(Cl)cc1Cl] | +++ | +++ | 440.1 |
| 1.022 | 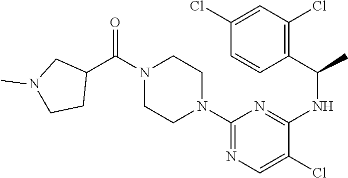 [SMILES: C[C@@H](Nc1nc(N2CCN(C(=O)C3CCN(C)C3)CC2)ncc1Cl)c1ccc(Cl)cc1Cl] | +++ | +++ | 497.2 |
| 1.023 | 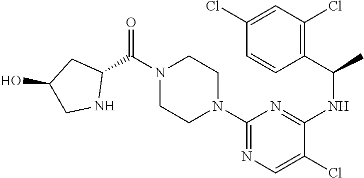 [SMILES: C[C@@H](Nc1nc(N2CCN(C(=O)[C@H]3C[C@H](O)CN3)CC2)ncc1Cl)c1ccc(Cl)cc1Cl] | +++ | +++ | 499.1 |
| 1.024 | 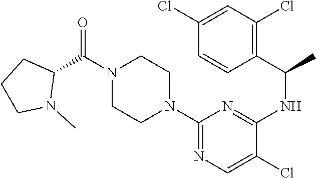 [SMILES: C[C@@H](Nc1nc(N2CCN(C(=O)[C@H]3CCCN3C)CC2)ncc1Cl)c1ccc(Cl)cc1Cl] | +++ | +++ | 497.1 |
| 1.025 | 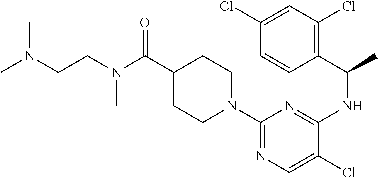 [SMILES: C[C@@H](Nc1nc(N2CCC(C(=O)N(C)CCN(C)C)CC2)ncc1Cl)c1ccc(Cl)cc1Cl] | +++ | +++ | 513.3 |
| 1.026 | 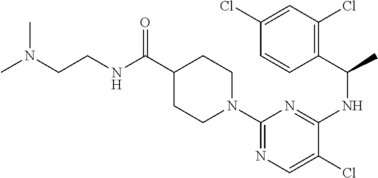 [SMILES: C[C@@H](Nc1nc(N2CCC(C(=O)NCCN(C)C)CC2)ncc1Cl)c1ccc(Cl)cc1Cl] | +++ | +++ | 499.1 |
| 1.027 | 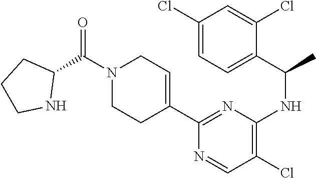 [SMILES: C[C@@H](Nc1nc(C2=CCN(C(=O)[C@H]3CCCN3)CC2)ncc1Cl)c1ccc(Cl)cc1Cl] | +++ | +++ | 480.1 |
| 1.028 | 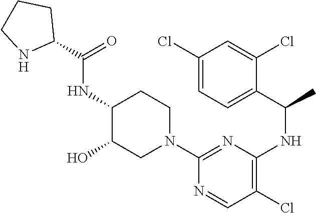 [SMILES: C[C@@H](Nc1nc(N2CC[C@@H](NC(=O)[C@H]3CCCN3)[C@@H](O)C2)ncc1Cl)c1ccc(Cl)cc1Cl] | +++ | +++ | 513.2 |
| 1.029 | 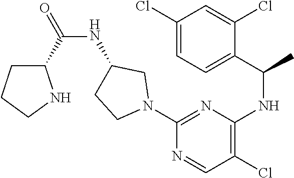 [SMILES: C[C@@H](Nc1nc(N2CC[C@H](NC(=O)[C@H]3CCCN3)C2)ncc1Cl)c1ccc(Cl)cc1Cl] | +++ | +++ | 483.1 |
| 1.030 | 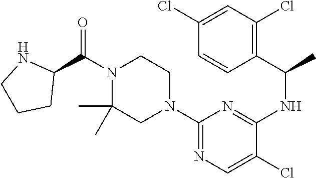 [SMILES: C[C@@H](Nc1nc(N2CCN(C(=O)[C@H]3CCCN3)C(C)(C)C2)ncc1Cl)c1ccc(Cl)cc1Cl] | + | ++ | 511.1 |
| 1.031 | 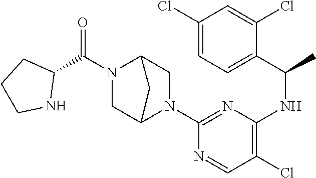 [SMILES: C[C@@H](Nc1nc(N2CC3CC2CN3C(=O)[C@H]2CCCN2)ncc1Cl)c1ccc(Cl)cc1Cl] | ++ | ++ | 495.1 |
| 1.032 | 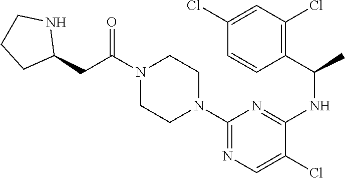 [SMILES: C[C@@H](Nc1nc(N2CCN(C(=O)C[C@H]3CCCN3)CC2)ncc1Cl)c1ccc(Cl)cc1Cl] | +++ | +++ | 497.2 |
| 1.033 | 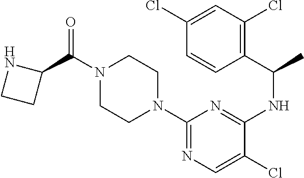 [SMILES: C[C@@H](Nc1nc(N2CCN(C(=O)[C@H]3CCN3)CC2)ncc1Cl)c1ccc(Cl)cc1Cl] | +++ | +++ | 469.1 |
| 1.034 | 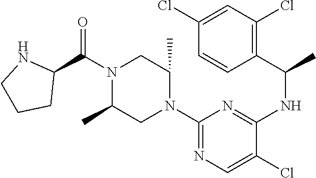 [SMILES: C[C@@H]1CN(c2ncc(Cl)c(N[C@H](C)c3ccc(Cl)cc3Cl)n2)[C@@H](C)CN1C(=O)[C@H]1CCCN1] | +++ | +++ | 511.1 |
| 1.035 | 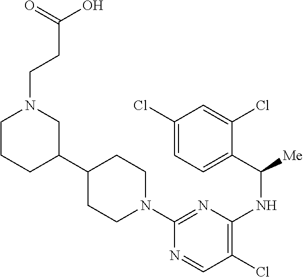 [SMILES: C[C@@H](Nc1nc(N2CCC(C3CCCN(CCC(=O)O)C3)CC2)ncc1Cl)c1ccc(Cl)cc1Cl] | +++ | +++ | 540.2 |
| 1.036 | 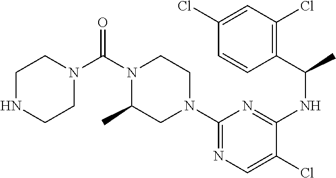 [SMILES: C[C@@H]1CN(c2ncc(Cl)c(N[C@H](C)c3ccc(Cl)cc3Cl)n2)CCN1C(=O)N1CCNCC1] | + | + | 512.1 |
| 1.037 | 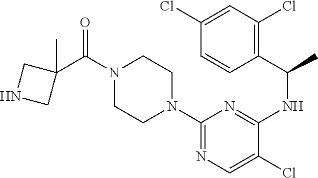 [SMILES: C[C@@H](Nc1nc(N2CCN(C(=O)C3(C)CNC3)CC2)ncc1Cl)c1ccc(Cl)cc1Cl] | ++ | ++ | 483.1 |
| 1.038 | 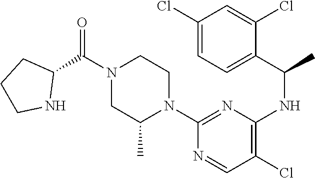 [SMILES: C[C@@H]1CN(C(=O)[C@H]2CCCN2)CCN1c1ncc(Cl)c(N[C@H](C)c2ccc(Cl)cc2Cl)n1] | +++ | +++ | 497.1 |
| 1.039 | 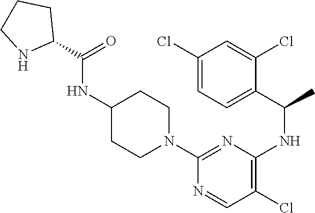 [SMILES: C[C@@H](Nc1nc(N2CCC(NC(=O)[C@H]3CCCN3)CC2)ncc1Cl)c1ccc(Cl)cc1Cl] | +++ | +++ | 497.1 |
| 1.040 | 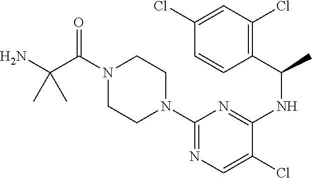 [SMILES: C[C@@H](Nc1nc(N2CCN(C(=O)C(C)(C)N)CC2)ncc1Cl)c1ccc(Cl)cc1Cl] | +++ | +++ | 471.1 |
| 1.041 | 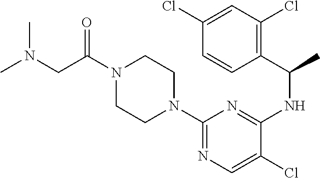 [SMILES: C[C@@H](Nc1nc(N2CCN(C(=O)CN(C)C)CC2)ncc1Cl)c1ccc(Cl)cc1Cl] | +++ | +++ | 471.1 |
| 1.042 | 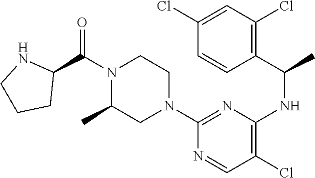 [SMILES: C[C@@H]1CN(c2ncc(Cl)c(N[C@H](C)c3ccc(Cl)cc3Cl)n2)CCN1C(=O)[C@H]1CCCN1] | +++ | +++ | 497.1 |
| 1.043 | 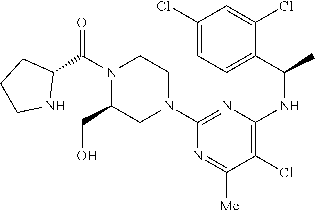 [SMILES: Cc1nc(N2CCN(C(=O)[C@H]3CCCN3)[C@H](CO)C2)nc(N[C@H](C)c2ccc(Cl)cc2Cl)c1Cl] | +++ | +++ | 527.2 |
| 1.044 | 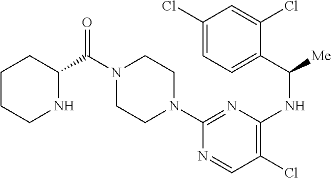 [SMILES: C[C@@H](Nc1nc(N2CCN(C(=O)[C@H]3CCCCN3)CC2)ncc1Cl)c1ccc(Cl)cc1Cl] | +++ | +++ | 497.2 |
| 1.045 | 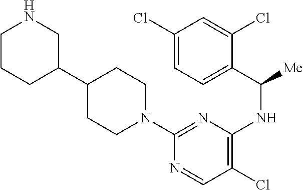 [SMILES: C[C@@H](Nc1nc(N2CCC(C3CCCNC3)CC2)ncc1Cl)c1ccc(Cl)cc1Cl] | +++ | +++ | 468.2 |
| 1.046 | 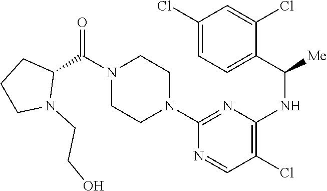 [SMILES: C[C@@H](Nc1nc(N2CCN(C(=O)[C@H]3CCCN3CCO)CC2)ncc1Cl)c1ccc(Cl)cc1Cl] | +++ | +++ | 527.2 |
| 1.047 | 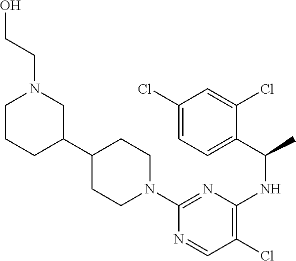 [SMILES: C[C@@H](Nc1nc(N2CCC(C3CCCN(CCO)C3)CC2)ncc1Cl)c1ccc(Cl)cc1Cl] | +++ | +++ | 512.3 |
| 1.048 | 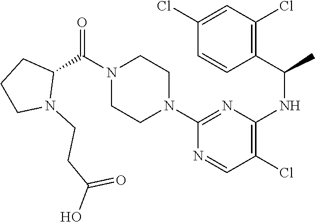 [SMILES: C[C@@H](Nc1nc(N2CCN(C(=O)[C@H]3CCCN3CCC(=O)O)CC2)ncc1Cl)c1ccc(Cl)cc1Cl] | +++ | +++ | 555.2 |
| 1.049 | 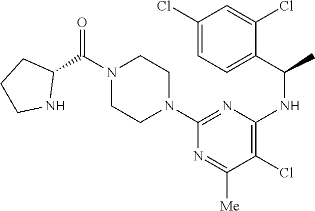 [SMILES: Cc1nc(N2CCN(C(=O)[C@H]3CCCN3)CC2)nc(N[C@H](C)c2ccc(Cl)cc2Cl)c1Cl] | +++ | +++ | 497.1 |
| 1.050 | 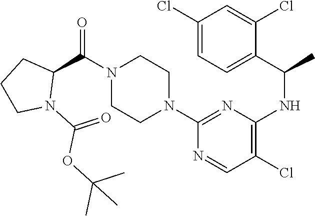 [SMILES: C[C@@H](Nc1nc(N2CCN(C(=O)[C@@H]3CCCN3C(=O)OC(C)(C)C)CC2)ncc1Cl)c1ccc(Cl)cc1Cl] | ++ | + | 583.2 |
| 1.051 | 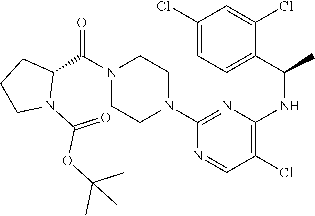 [SMILES: C[C@@H](Nc1nc(N2CCN(C(=O)[C@H]3CCCN3C(=O)OC(C)(C)C)CC2)ncc1Cl)c1ccc(Cl)cc1Cl] | ++ | ++ | 583.2 |
| 1.052 | 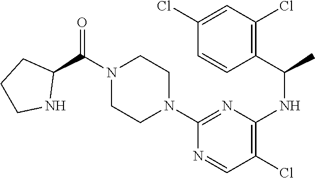 [SMILES: C[C@@H](Nc1nc(N2CCN(C(=O)[C@@H]3CCCN3)CC2)ncc1Cl)c1ccc(Cl)cc1Cl] | +++ | +++ | 483.1 |
| 1.053 | 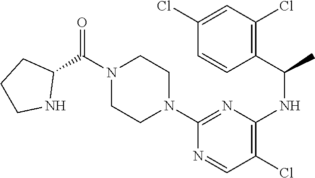 [SMILES: C[C@@H](Nc1nc(N2CCN(C(=O)[C@H]3CCCN3)CC2)ncc1Cl)c1ccc(Cl)cc1Cl] | +++ | +++ | 483.1 |
| 1.054 | 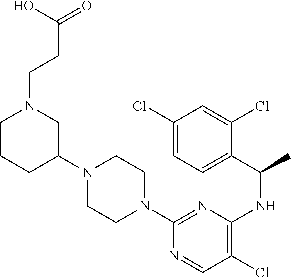 [SMILES: C[C@@H](Nc1nc(N2CCN(C3CCCN(CCC(=O)O)C3)CC2)ncc1Cl)c1ccc(Cl)cc1Cl] | +++ | +++ | 541.2 |
| 1.055 | 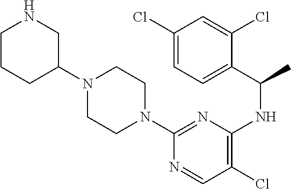 [SMILES: C[C@@H](Nc1nc(N2CCN(C3CCCNC3)CC2)ncc1Cl)c1ccc(Cl)cc1Cl] | +++ | +++ | 469.1 |
| 1.056 | 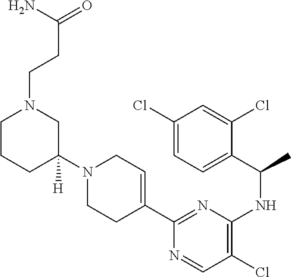 [SMILES: C[C@@H](Nc1nc(C2=CCN([C@H]3CCCN(CCC(N)=O)C3)CC2)ncc1Cl)c1ccc(Cl)cc1Cl] | +++ | +++ | 537.2 |
| 1.057 | 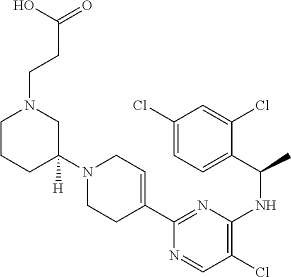 [SMILES: C[C@@H](Nc1nc(C2=CCN([C@H]3CCCN(CCC(=O)O)C3)CC2)ncc1Cl)c1ccc(Cl)cc1Cl] | +++ | +++ | 538.2 |
| 1.058 | 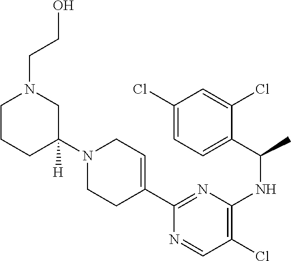 [SMILES: C[C@@H](Nc1nc(C2=CCN([C@H]3CCCN(CCO)C3)CC2)ncc1Cl)c1ccc(Cl)cc1Cl] | +++ | +++ | 510.2 |
| 1.059 | 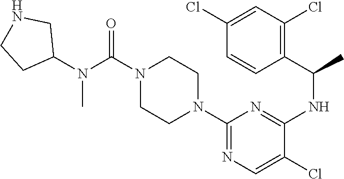 [SMILES: C[C@@H](Nc1nc(N2CCN(C(=O)N(C)C3CCNC3)CC2)ncc1Cl)c1ccc(Cl)cc1Cl] | +++ | +++ | 512.2 |
| 1.060 | 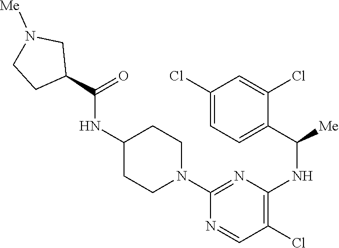 [SMILES: C[C@@H](Nc1nc(N2CCC(NC(=O)[C@H]3CCN(C)C3)CC2)ncc1Cl)c1ccc(Cl)cc1Cl] | +++ | +++ | 511.2 |
| 1.061 | 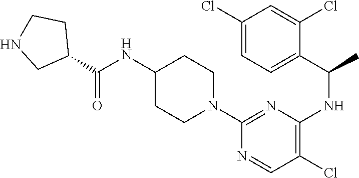 [SMILES: C[C@@H](Nc1nc(N2CCC(NC(=O)[C@H]3CCNC3)CC2)ncc1Cl)c1ccc(Cl)cc1Cl] | +++ | +++ | 497.2 |
| 1.062 | 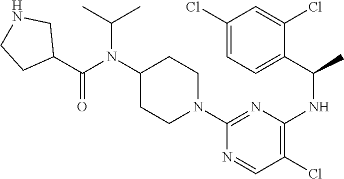 [SMILES: CC(C)N(C(=O)C1CCNC1)C1CCN(c2ncc(Cl)c(N[C@H](C)c3ccc(Cl)cc3Cl)n2)CC1] | + | + | 538.2 |
| 1.063 | 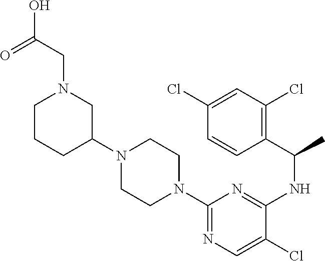 [SMILES: C[C@@H](Nc1nc(N2CCN(C3CCCN(CC(=O)O)C3)CC2)ncc1Cl)c1ccc(Cl)cc1Cl] | +++ | +++ | 527.2 |
| 1.064 | 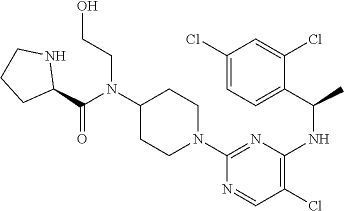 [SMILES: C[C@@H](Nc1nc(N2CCC(N(CCO)C(=O)[C@H]3CCCN3)CC2)ncc1Cl)c1ccc(Cl)cc1Cl] | +++ | +++ | 541.2 |
| 1.065 | 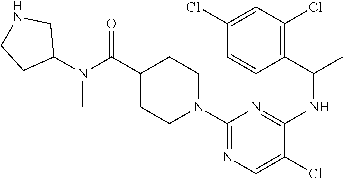 [SMILES: CC(Nc1nc(N2CCC(C(=O)N(C)C3CCNC3)CC2)ncc1Cl)c1ccc(Cl)cc1Cl] | +++ | +++ | 511.2 |
| 1.066 | 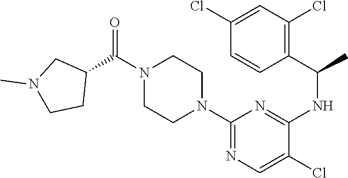 [SMILES: C[C@@H](Nc1nc(N2CCN(C(=O)[C@@H]3CCN(C)C3)CC2)ncc1Cl)c1ccc(Cl)cc1Cl] | +++ | +++ | 497.1 |
| 1.067 | 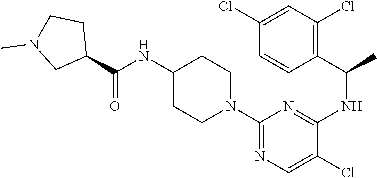 [SMILES: C[C@@H](Nc1nc(N2CCC(NC(=O)[C@@H]3CCN(C)C3)CC2)ncc1Cl)c1ccc(Cl)cc1Cl] | +++ | ++ | 511.1 |
| 1.068 | 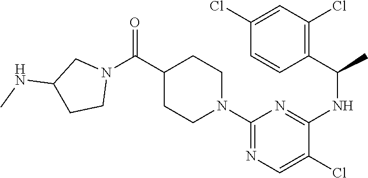 [SMILES: CNC1CCN(C(=O)C2CCN(c3ncc(Cl)c(N[C@H](C)c4ccc(Cl)cc4Cl)n3)CC2)C1] | +++ | +++ | 511.2 |
| 1.069 | 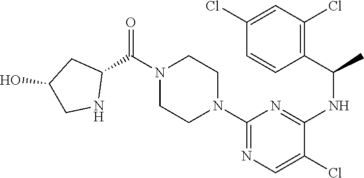 [SMILES: C[C@@H](Nc1nc(N2CCN(C(=O)[C@H]3C[C@@H](O)CN3)CC2)ncc1Cl)c1ccc(Cl)cc1Cl] | +++ | +++ | 499.1 |
| 1.070 | 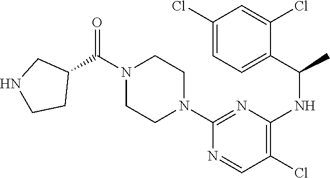 [SMILES: C[C@@H](Nc1nc(N2CCN(C(=O)[C@@H]3CCNC3)CC2)ncc1Cl)c1ccc(Cl)cc1Cl] | +++ | +++ | 483.1 |
| 1.071 | 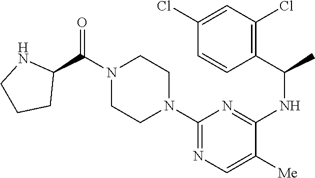 [SMILES: Cc1cnc(N2CCN(C(=O)[C@H]3CCCN3)CC2)nc1N[C@H](C)c1ccc(Cl)cc1Cl] | +++ | +++ | 463.2 |
| 1.072 | 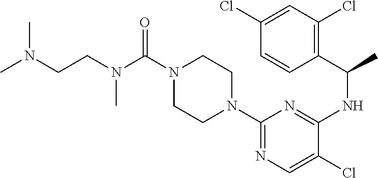 [SMILES: C[C@@H](Nc1nc(N2CCN(C(=O)N(C)CCN(C)C)CC2)ncc1Cl)c1ccc(Cl)cc1Cl] | +++ | +++ | 516.2 |
| 1.073 | 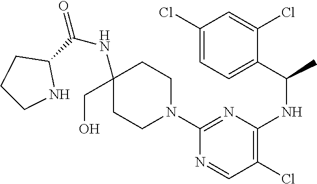 [SMILES: C[C@@H](Nc1nc(N2CCC(CO)(NC(=O)[C@H]3CCCN3)CC2)ncc1Cl)c1ccc(Cl)cc1Cl] | +++ | +++ | 527.2 |
| 1.074 | 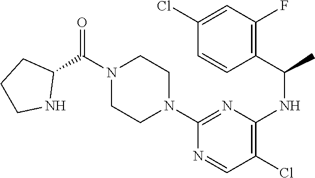 [SMILES: C[C@@H](Nc1nc(N2CCN(C(=O)[C@H]3CCCN3)CC2)ncc1Cl)c1ccc(Cl)cc1F] | +++ | +++ | 467.1 |
| 1.075 | 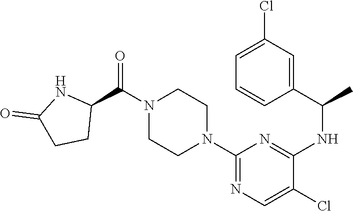 [SMILES: C[C@@H](Nc1nc(N2CCN(C(=O)[C@H]3CCC(=O)N3)CC2)ncc1Cl)c1cccc(Cl)c1] | + | ++ | 463.1 |
| 1.076 | 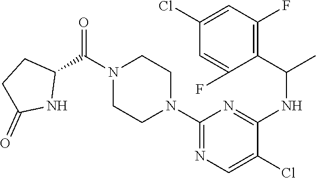 [SMILES: CC(Nc1nc(N2CCN(C(=O)[C@H]3CCC(=O)N3)CC2)ncc1Cl)c1c(F)cc(Cl)cc1F] | +++ | +++ | 499.1 |
| 1.077 | 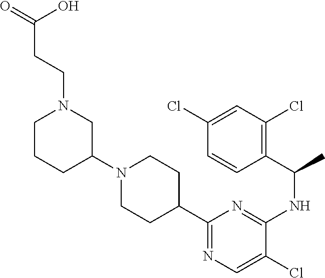 [SMILES: C[C@@H](Nc1nc(C2CCN(C3CCCN(CCC(=O)O)C3)CC2)ncc1Cl)c1ccc(Cl)cc1Cl] | +++ | +++ | 540.2 |
| 1.078 | 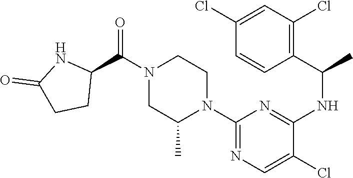 [SMILES: C[C@@H]1CN(C(=O)[C@H]2CCC(=O)N2)CCN1c1ncc(Cl)c(N[C@H](C)c2ccc(Cl)cc2Cl)n1] | +++ | +++ | 511.1 |
| 1.079 | 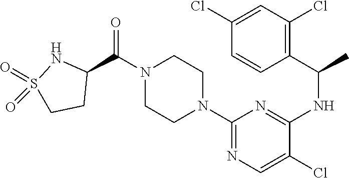 [SMILES: C[C@@H](Nc1nc(N2CCN(C(=O)[C@H]3CCS(=O)(=O)N3)CC2)ncc1Cl)c1ccc(Cl)cc1Cl] | +++ | +++ | 533.1 |
| 1.080 | 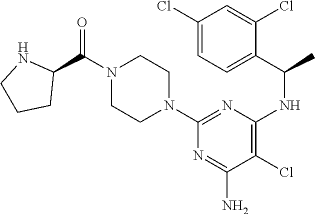 [SMILES: C[C@@H](Nc1nc(N2CCN(C(=O)[C@H]3CCCN3)CC2)nc(N)c1Cl)c1ccc(Cl)cc1Cl] | +++ | +++ | 498.1 |
| 1.081 | 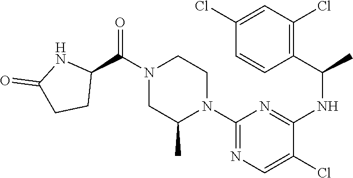 [SMILES: C[C@@H](Nc1nc(N2CCN(C(=O)[C@H]3CCC(=O)N3)C[C@@H]2C)ncc1Cl)c1ccc(Cl)cc1Cl] | +++ | +++ | 511.1 |
| 1.082 | 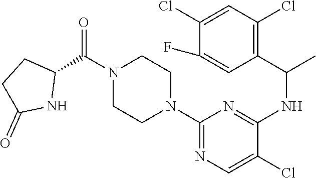 [SMILES: CC(Nc1nc(N2CCN(C(=O)[C@H]3CCC(=O)N3)CC2)ncc1Cl)c1cc(F)c(Cl)cc1Cl] | +++ | +++ | 515.1 |
| 1.083 | 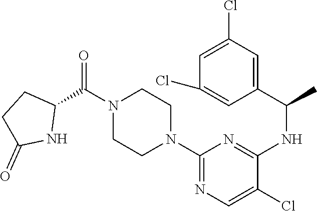 [SMILES: C[C@@H](Nc1nc(N2CCN(C(=O)[C@H]3CCC(=O)N3)CC2)ncc1Cl)c1cc(Cl)cc(Cl)c1] | + | + | 497.1 |
| 1.084 | 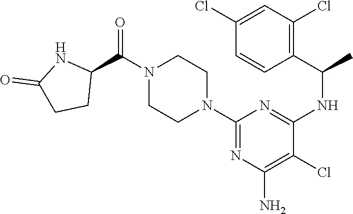 [SMILES: C[C@@H](Nc1nc(N2CCN(C(=O)[C@H]3CCC(=O)N3)CC2)nc(N)c1Cl)c1ccc(Cl)cc1Cl] | | +++ | 512.2 |
| 1.085 | 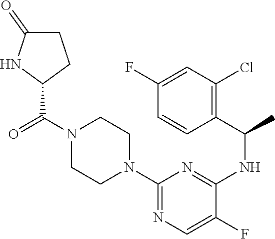 [SMILES: C[C@@H](Nc1nc(N2CCN(C(=O)[C@H]3CCC(=O)N3)CC2)ncc1F)c1ccc(F)cc1Cl] | | +++ | 465.1 |
| 1.086 | 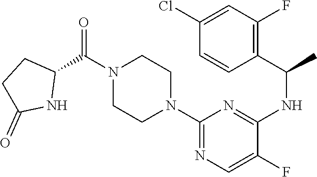 [SMILES: C[C@@H](Nc1nc(N2CCN(C(=O)[C@H]3CCC(=O)N3)CC2)ncc1F)c1ccc(Cl)cc1F] | | +++ | 465.2 |
| 1.087 | 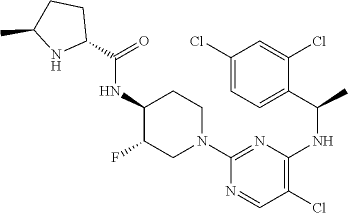 [SMILES: C[C@H]1CC[C@H](C(=O)N[C@H]2CCN(c3ncc(Cl)c(N[C@H](C)c4ccc(Cl)cc4Cl)n3)C[C@@H]2F)N1] | | +++ | 529.1 |
| 1.088 | 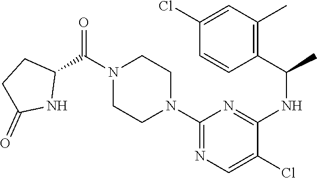 [SMILES: Cc1cc(Cl)ccc1[C@@H](C)Nc1nc(N2CCN(C(=O)[C@H]3CCC(=O)N3)CC2)ncc1Cl] | | ++ | 477.2 |
| 1.089 | 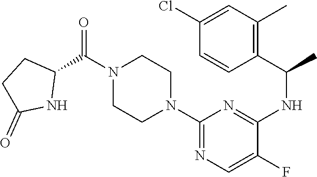 [SMILES: Cc1cc(Cl)ccc1[C@@H](C)Nc1nc(N2CCN(C(=O)[C@H]3CCC(=O)N3)CC2)ncc1F] | | ++ | 461.2 |
| 1.090 | 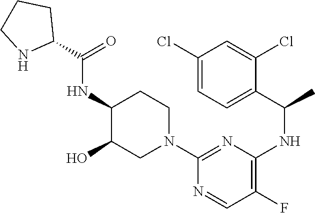 [SMILES: C[C@@H](Nc1nc(N2CC[C@H](NC(=O)[C@H]3CCCN3)[C@H](O)C2)ncc1F)c1ccc(Cl)cc1Cl] | | +++ | 497.2 |
| 1.091 | 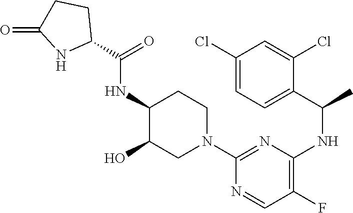 [SMILES: C[C@@H](Nc1nc(N2CC[C@H](NC(=O)[C@H]3CCC(=O)N3)[C@H](O)C2)ncc1F)c1ccc(Cl)cc1Cl] | | + | 511.1 |
| 1.092 | 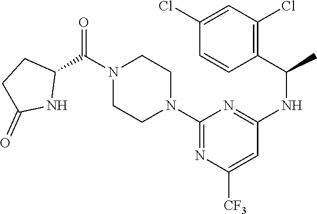 [SMILES: C[C@@H](Nc1cc(C(F)(F)F)nc(N2CCN(C(=O)[C@H]3CCC(=O)N3)CC2)n1)c1ccc(Cl)cc1Cl] | | ++ | 531.1 |
| 1.093 | 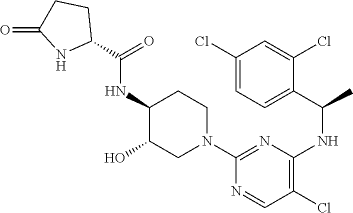 [SMILES: C[C@@H](Nc1nc(N2CC[C@H](NC(=O)[C@H]3CCC(=O)N3)[C@@H](O)C2)ncc1Cl)c1ccc(Cl)cc1Cl] | | ++ | 527.1 |
| 1.094 | 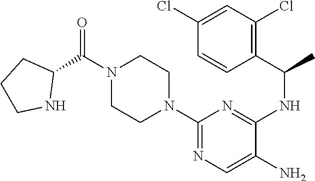 [SMILES: C[C@@H](Nc1nc(N2CCN(C(=O)[C@H]3CCCN3)CC2)ncc1N)c1ccc(Cl)cc1Cl] | | +++ | 464.2 |
| 1.095 | 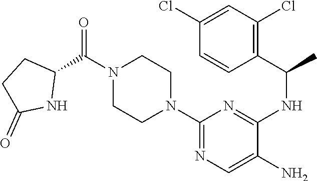 [SMILES: C[C@@H](Nc1nc(N2CCN(C(=O)[C@H]3CCC(=O)N3)CC2)ncc1N)c1ccc(Cl)cc1Cl] | | +++ | 478.1 |
| 1.096 | 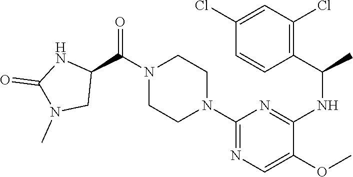 [SMILES: COc1cnc(N2CCN(C(=O)[C@H]3CN(C)C(=O)N3)CC2)nc1N[C@H](C)c1ccc(Cl)cc1Cl] | | +++ | 508.2 |
| 1.097 | 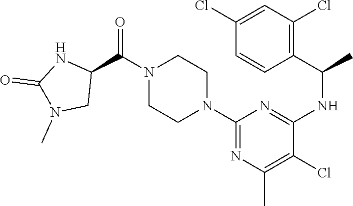 [SMILES: Cc1nc(N2CCN(C(=O)[C@H]3CN(C)C(=O)N3)CC2)nc(N[C@H](C)c2ccc(Cl)cc2Cl)c1Cl] | | +++ | 526.1 |
| 1.098 | 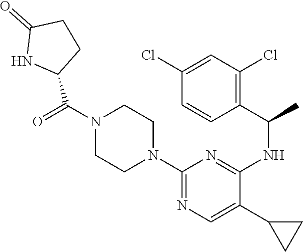 [SMILES: C[C@@H](Nc1nc(N2CCN(C(=O)[C@H]3CCC(=O)N3)CC2)ncc1C1CC1)c1ccc(Cl)cc1Cl] | | ++ | 503.1 |
| 1.099 | 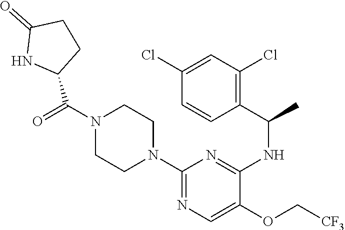 [SMILES: C[C@@H](Nc1nc(N2CCN(C(=O)[C@H]3CCC(=O)N3)CC2)ncc1OCC(F)(F)F)c1ccc(Cl)cc1Cl] | | ++ | 561.1 |
| 1.100 | 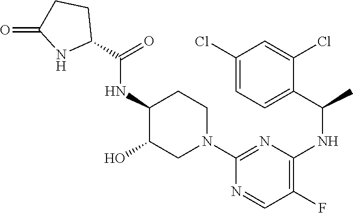 [SMILES: C[C@@H](Nc1nc(N2CC[C@H](NC(=O)[C@H]3CCC(=O)N3)[C@@H](O)C2)ncc1F)c1ccc(Cl)cc1Cl] | | + | 511.1 |
| 1.101 | 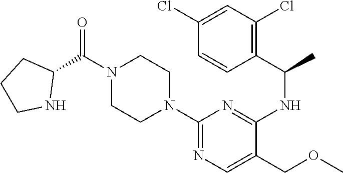 [SMILES: COCc1cnc(N2CCN(C(=O)[C@H]3CCCN3)CC2)nc1N[C@H](C)c1ccc(Cl)cc1Cl] | | +++ | 493.2 |
| 1.102 | 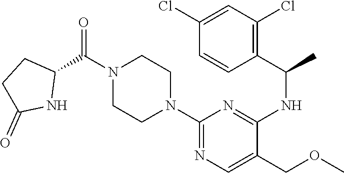 [SMILES: COCc1cnc(N2CCN(C(=O)[C@H]3CCC(=O)N3)CC2)nc1N[C@H](C)c1ccc(Cl)cc1Cl] | | +++ | 507.1 |
| 1.103 | 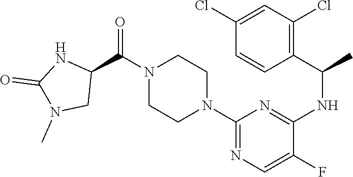 [SMILES: C[C@@H](Nc1nc(N2CCN(C(=O)[C@H]3CN(C)C(=O)N3)CC2)ncc1F)c1ccc(Cl)cc1Cl] | | +++ | 496.1 |
| 1.104 | 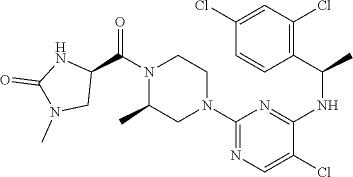 [SMILES: C[C@@H]1CN(c2ncc(Cl)c(N[C@H](C)c3ccc(Cl)cc3Cl)n2)CCN1C(=O)[C@H]1CN(C)C(=O)N1] | | +++ | 526.2 |
| 1.105 | 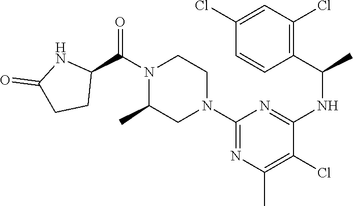 [SMILES: Cc1nc(N2CCN(C(=O)[C@H]3CCC(=O)N3)[C@H](C)C2)nc(N[C@H](C)c2ccc(Cl)cc2Cl)c1Cl] | | +++ | 525.2 |
| 1.106 | 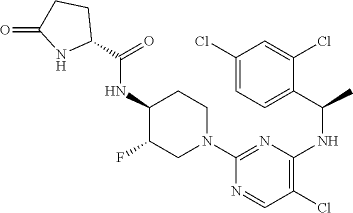 [SMILES: C[C@@H](Nc1nc(N2CC[C@H](NC(=O)[C@H]3CCC(=O)N3)[C@@H](F)C2)ncc1Cl)c1ccc(Cl)cc1Cl] | | ++ | 530.1 |
| 1.107 | 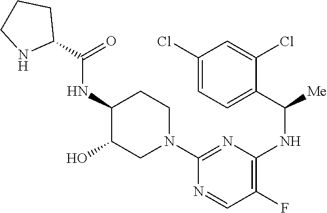 [SMILES: C[C@@H](Nc1nc(N2CC[C@H](NC(=O)[C@H]3CCCN3)[C@@H](O)C2)ncc1F)c1ccc(Cl)cc1Cl] | | +++ | 497.1 |
| 1.108 | 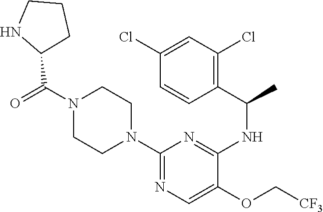 [SMILES: C[C@@H](Nc1nc(N2CCN(C(=O)[C@H]3CCCN3)CC2)ncc1OCC(F)(F)F)c1ccc(Cl)cc1Cl] | | +++ | 547.2 |
| 1.109 | 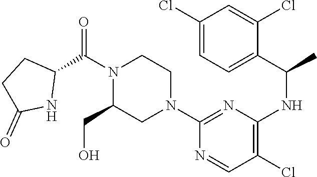 [SMILES: C[C@@H](Nc1nc(N2CCN(C(=O)[C@H]3CCC(=O)N3)[C@H](CO)C2)ncc1Cl)c1ccc(Cl)cc1Cl] | | +++ | 527.1 |
| 1.110 | 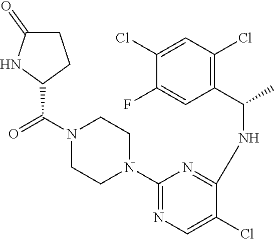 | + | + | 515.1 |
| 1.111 | 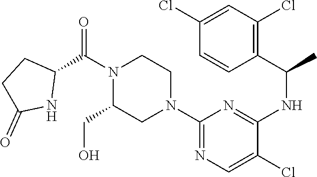 [SMILES: C[C@@H](Nc1nc(N2CCN(C(=O)[C@H]3CCC(=O)N3)[C@@H](CO)C2)ncc1Cl)c1ccc(Cl)cc1Cl] | | +++ | 527.1 |
| 1.112 | 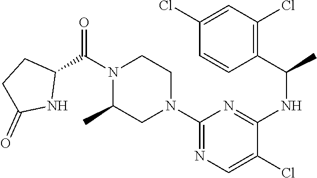 [SMILES: C[C@@H]1CN(c2ncc(Cl)c(N[C@H](C)c3ccc(Cl)cc3Cl)n2)CCN1C(=O)[C@H]1CCC(=O)N1] | | +++ | 511.1 |
| 1.113 | 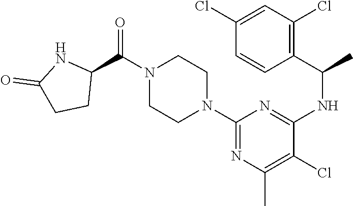 [SMILES: Cc1nc(N2CCN(C(=O)[C@H]3CCC(=O)N3)CC2)nc(N[C@H](C)c2ccc(Cl)cc2Cl)c1Cl] | +++ | +++ | 511.1 |
| 1.114 | 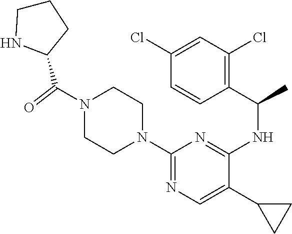 [SMILES: C[C@@H](Nc1nc(N2CCN(C(=O)[C@H]3CCCN3)CC2)ncc1C1CC1)c1ccc(Cl)cc1Cl] | | +++ | 489.2 |
| 1.115 | 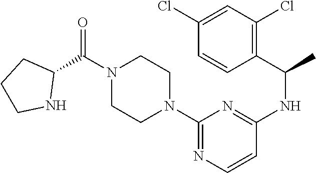 [SMILES: C[C@@H](Nc1ccnc(N2CCN(C(=O)[C@H]3CCCN3)CC2)n1)c1ccc(Cl)cc1Cl] | | +++ | 449.2 |
| 1.116 | 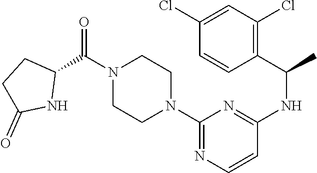 [SMILES: C[C@@H](Nc1ccnc(N2CCN(C(=O)[C@H]3CCC(=O)N3)CC2)n1)c1ccc(Cl)cc1Cl] | | +++ | 463.1 |
| 1.117 | 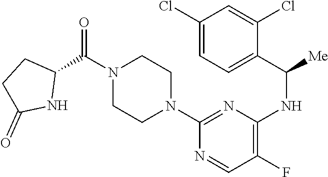 [SMILES: C[C@@H](Nc1nc(N2CCN(C(=O)[C@H]3CCC(=O)N3)CC2)ncc1F)c1ccc(Cl)cc1Cl] | | +++ | 481.1 |
| 1.118 | 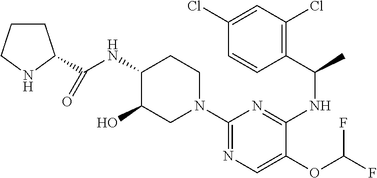 [SMILES: C[C@@H](Nc1nc(N2CC[C@@H](NC(=O)[C@H]3CCCN3)[C@H](O)C2)ncc1OC(F)F)c1ccc(Cl)cc1Cl] | | +++ | 545.2 |
| 1.119 | 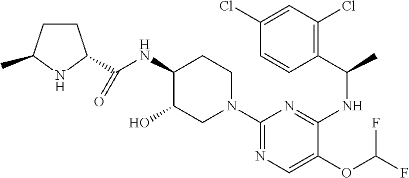 [SMILES: C[C@H]1CC[C@H](C(=O)N[C@H]2CCN(c3ncc(OC(F)F)c(N[C@H](C)c4ccc(Cl)cc4Cl)n3)C[C@@H]2O)N1] | | +++ | 559.2 |
| 1.120 | 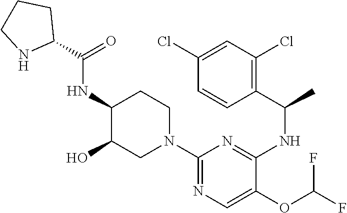 [SMILES: C[C@@H](Nc1nc(N2CC[C@H](NC(=O)[C@H]3CCCN3)[C@H](O)C2)ncc1OC(F)F)c1ccc(Cl)cc1Cl] | | +++ | 545.2 |
| 1.121 | 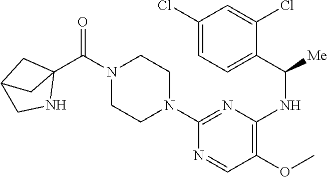 [SMILES: COc1cnc(N2CCN(C(=O)C34CC(CN3)C4)CC2)nc1N[C@H](C)c1ccc(Cl)cc1Cl] | | +++ | 491.2 |
| 1.122 | 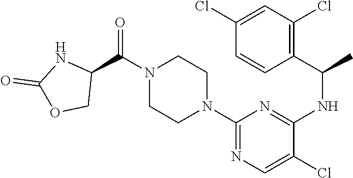 [SMILES: C[C@@H](Nc1nc(N2CCN(C(=O)[C@H]3COC(=O)N3)CC2)ncc1Cl)c1ccc(Cl)cc1Cl] | | +++ | 499.1 |
| 1.123 | 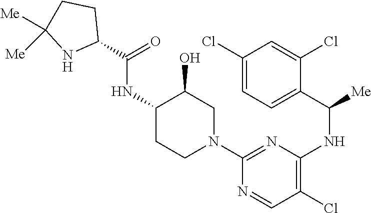 [SMILES: C[C@@H](Nc1nc(N2CC[C@H](NC(=O)[C@H]3CCC(C)(C)N3)[C@@H](O)C2)ncc1Cl)c1ccc(Cl)cc1Cl] | | +++ | 541.2 |
| 1.124 | 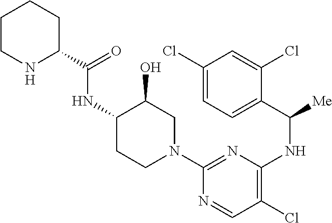 [SMILES: C[C@@H](Nc1nc(N2CC[C@H](NC(=O)[C@H]3CCCCN3)[C@@H](O)C2)ncc1Cl)c1ccc(Cl)cc1Cl] | | +++ | 527.1 |
| 1.125 | 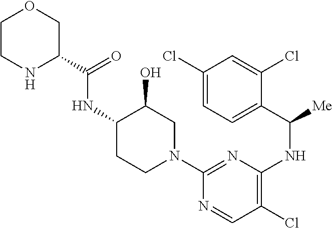 [SMILES: C[C@@H](Nc1nc(N2CC[C@H](NC(=O)[C@H]3COCCN3)[C@@H](O)C2)ncc1Cl)c1ccc(Cl)cc1Cl] | | ++ | 529.1 |
| 1.126 | 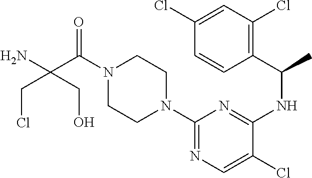 [SMILES: C[C@@H](Nc1nc(N2CCN(C(=O)C(N)(CO)CCl)CC2)ncc1Cl)c1ccc(Cl)cc1Cl] | | ++ | 521.1 |
| 1.127 | 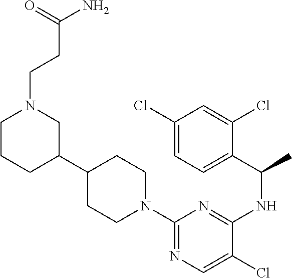 [SMILES: C[C@@H](Nc1nc(N2CCC(C3CCCN(CCC(N)=O)C3)CC2)ncc1Cl)c1ccc(Cl)cc1Cl] | | +++ | 539.2 |
| 1.128 | 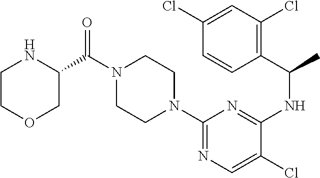 [SMILES: C[C@@H](Nc1nc(N2CCN(C(=O)[C@@H]3COCCN3)CC2)ncc1Cl)c1ccc(Cl)cc1Cl] | | ++ | 499.1 |
| 1.129 | 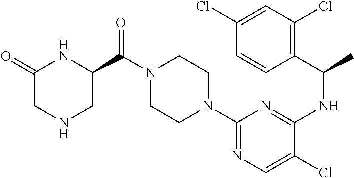 [SMILES: C[C@@H](Nc1nc(N2CCN(C(=O)[C@H]3CNCC(=O)N3)CC2)ncc1Cl)c1ccc(Cl)cc1Cl] | | +++ | 512.1 |
| 1.130 | 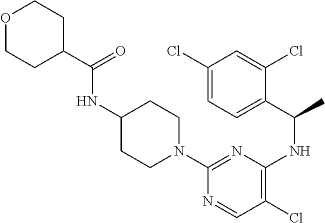 [SMILES: C[C@@H](Nc1nc(N2CCC(NC(=O)C3CCOCC3)CC2)ncc1Cl)c1ccc(Cl)cc1Cl] | ++ | + | 512.2 |
| 1.131 | 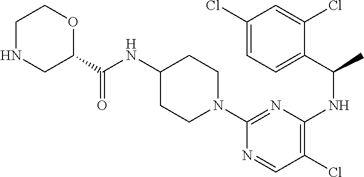 [SMILES: C[C@@H](Nc1nc(N2CCC(NC(=O)[C@@H]3CNCCO3)CC2)ncc1Cl)c1ccc(Cl)cc1Cl] | ++ | + | 513.1 |
| 1.132 | 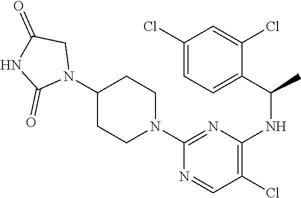 [SMILES: C[C@@H](Nc1nc(N2CCC(N3CC(=O)NC3=O)CC2)ncc1Cl)c1ccc(Cl)cc1Cl] | + | + | 483.1 |
| 1.133 | 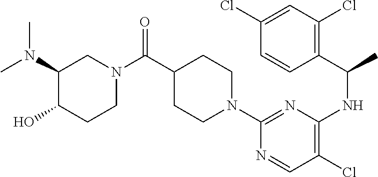 [SMILES: C[C@@H](Nc1nc(N2CCC(C(=O)N3CC[C@H](O)[C@@H](N(C)C)C3)CC2)ncc1Cl)c1ccc(Cl)cc1Cl] | ++ | + | 555.1 |
| 1.134 | 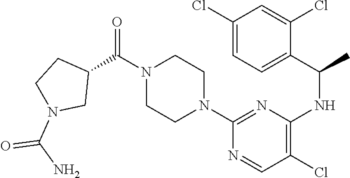 [SMILES: C[C@@H](Nc1nc(N2CCN(C(=O)[C@H]3CCN(C(N)=O)C3)CC2)ncc1Cl)c1ccc(Cl)cc1Cl] | +++ | +++ | 526.2 |
| 1.135 | 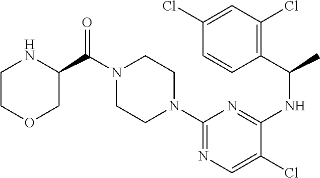 [SMILES: C[C@@H](Nc1nc(N2CCN(C(=O)[C@H]3COCCN3)CC2)ncc1Cl)c1ccc(Cl)cc1Cl] | +++ | +++ | 499.1 |
| 1.136 | 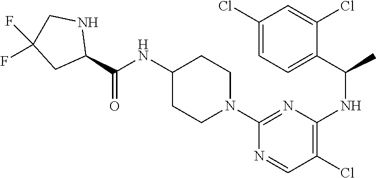 [SMILES: C[C@@H](Nc1nc(N2CCC(NC(=O)[C@H]3CC(F)(F)CN3)CC2)ncc1Cl)c1ccc(Cl)cc1Cl] | + | + | 533.1 |
| 1.137 | 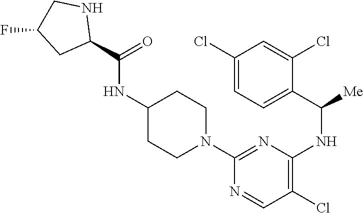 [SMILES: C[C@@H](Nc1nc(N2CCC(NC(=O)[C@H]3C[C@H](F)CN3)CC2)ncc1Cl)c1ccc(Cl)cc1Cl] | | ++ | 515.2 |
| 1.138 | 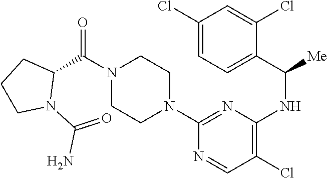 [SMILES: C[C@@H](Nc1nc(N2CCN(C(=O)[C@H]3CCCN3C(N)=O)CC2)ncc1Cl)c1ccc(Cl)cc1Cl] | | ++ | 526.1 |
| 1.139 | 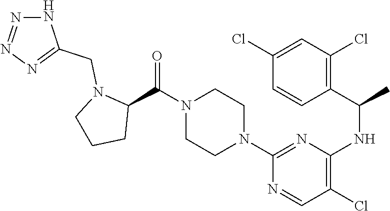 [SMILES: C[C@@H](Nc1nc(N2CCN(C(=O)[C@H]3CCCN3Cc3nnn[nH]3)CC2)ncc1Cl)c1ccc(Cl)cc1Cl] | | +++ | 565.2 |
| 1.140 | 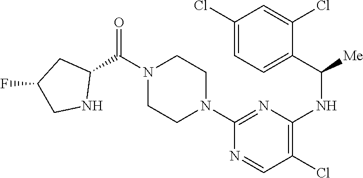 [SMILES: C[C@@H](Nc1nc(N2CCN(C(=O)[C@H]3C[C@@H](F)CN3)CC2)ncc1Cl)c1ccc(Cl)cc1Cl] | | +++ | 501.1 |
| 1.141 | 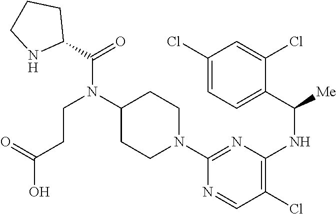 [SMILES: C[C@@H](Nc1nc(N2CCC(N(CCC(=O)O)C(=O)[C@H]3CCCN3)CC2)ncc1Cl)c1ccc(Cl)cc1Cl] | | + | 569.2 |
| 1.142 | 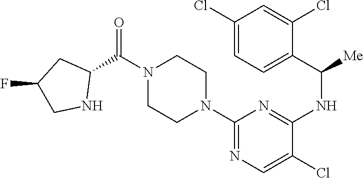 [SMILES: C[C@@H](Nc1nc(N2CCN(C(=O)[C@H]3C[C@H](F)CN3)CC2)ncc1Cl)c1ccc(Cl)cc1Cl] | | +++ | 501.1 |
| 1.143 | 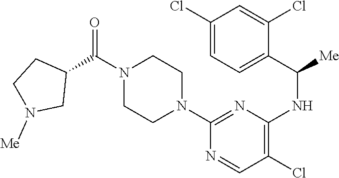 [SMILES: C[C@@H](Nc1nc(N2CCN(C(=O)[C@H]3CCN(C)C3)CC2)ncc1Cl)c1ccc(Cl)cc1Cl] | | +++ | 497.1 |
| 1.144 | 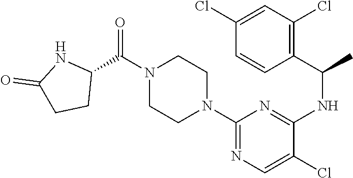 [SMILES: C[C@@H](Nc1nc(N2CCN(C(=O)[C@@H]3CCC(=O)N3)CC2)ncc1Cl)c1ccc(Cl)cc1Cl] | | ++ | 497.1 |
| 1.145 | 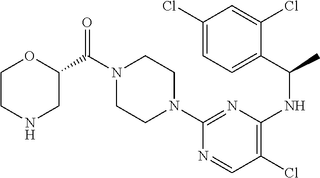 [SMILES: C[C@@H](Nc1nc(N2CCN(C(=O)[C@@H]3CNCCO3)CC2)ncc1Cl)c1ccc(Cl)cc1Cl] | | +++ | 499.1 |
| 1.146 | 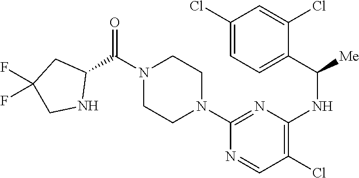 [SMILES: C[C@@H](Nc1nc(N2CCN(C(=O)[C@H]3CC(F)(F)CN3)CC2)ncc1Cl)c1ccc(Cl)cc1Cl] | | ++ | 519.1 |
| 1.147 | 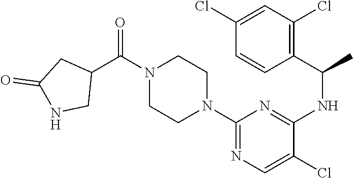 [SMILES: C[C@@H](Nc1nc(N2CCN(C(=O)C3CNC(=O)C3)CC2)ncc1Cl)c1ccc(Cl)cc1Cl] | | +++ | 497.1 |
| 1.148 | 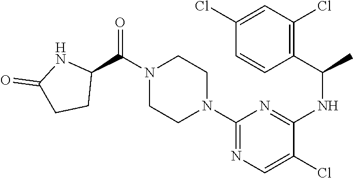 [SMILES: C[C@@H](Nc1nc(N2CCN(C(=O)[C@H]3CCC(=O)N3)CC2)ncc1Cl)c1ccc(Cl)cc1Cl] | +++ | +++ | 497.1 |
| 1.149 | 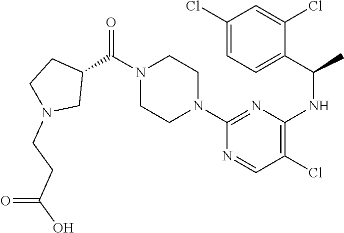 [SMILES: C[C@@H](Nc1nc(N2CCN(C(=O)[C@H]3CCN(CCC(=O)O)C3)CC2)ncc1Cl)c1ccc(Cl)cc1Cl] | +++ | +++ | 552.2 |
| 1.150 | 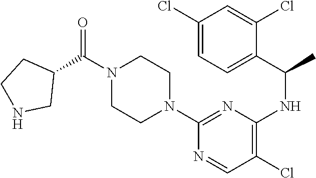 [SMILES: C[C@@H](Nc1nc(N2CCN(C(=O)[C@H]3CCNC3)CC2)ncc1Cl)c1ccc(Cl)cc1Cl] | +++ | +++ | 483.1 |
| 1.151 | 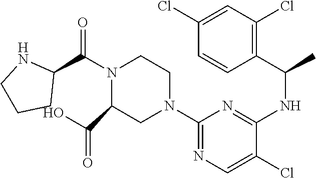 [SMILES: C[C@@H](Nc1nc(N2CCN(C(=O)[C@H]3CCCN3)[C@H](C(=O)O)C2)ncc1Cl)c1ccc(Cl)cc1Cl] | +++ | +++ | 527.1 |
| 1.152 | 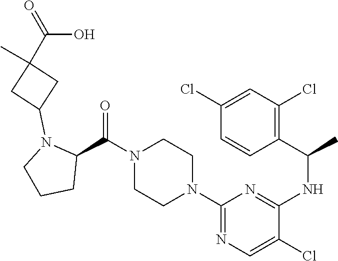 [SMILES: C[C@@H](Nc1nc(N2CCN(C(=O)[C@H]3CCCN3C3CC(C)(C(=O)O)C3)CC2)ncc1Cl)c1ccc(Cl)cc1Cl] | ++ | + | 595.3 |
| 1.153 | 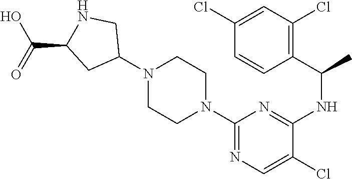 [SMILES: C[C@@H](Nc1nc(N2CCN(C3CN[C@H](C(=O)O)C3)CC2)ncc1Cl)c1ccc(Cl)cc1Cl] | + | + | 499.1 |
| 1.154 | 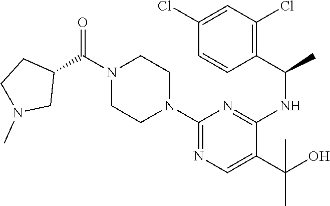 [SMILES: C[C@@H](Nc1nc(N2CCN(C(=O)[C@H]3CCN(C)C3)CC2)ncc1C(C)(C)O)c1ccc(Cl)cc1Cl] | | ++ | 521.2 |
| 1.155 | 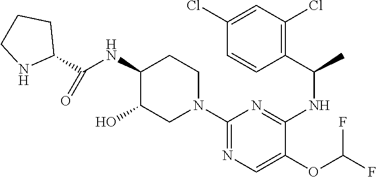 [SMILES: C[C@@H](Nc1nc(N2CC[C@H](NC(=O)[C@H]3CCCN3)[C@@H](O)C2)ncc1OC(F)F)c1ccc(Cl)cc1Cl] | | +++ | 545.2 |
| 1.156 | 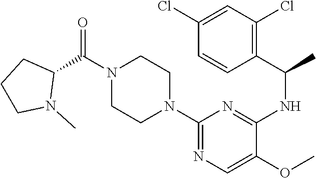 [SMILES: COc1cnc(N2CCN(C(=O)[C@H]3CCCN3C)CC2)nc1N[C@H](C)c1ccc(Cl)cc1Cl] | | +++ | 493.2 |
| 1.157 | 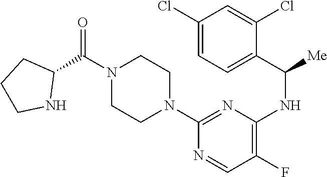 [SMILES: C[C@@H](Nc1nc(N2CCN(C(=O)[C@H]3CCCN3)CC2)ncc1F)c1ccc(Cl)cc1Cl] | | +++ | 467.1 |
| 1.158 | 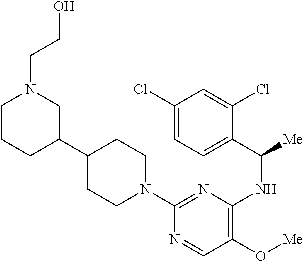 [SMILES: COc1cnc(N2CCC(C3CCCN(CCO)C3)CC2)nc1N[C@H](C)c1ccc(Cl)cc1Cl] | | +++ | 508.2 |
| 1.159 | 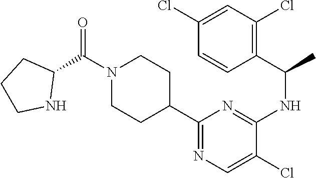 [SMILES: C[C@@H](Nc1nc(C2CCN(C(=O)[C@H]3CCCN3)CC2)ncc1Cl)c1ccc(Cl)cc1Cl] | | +++ | 482.1 |
| 1.160 | 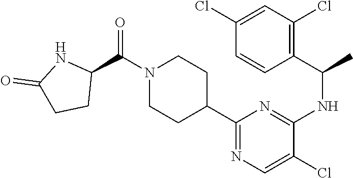 [SMILES: C[C@@H](Nc1nc(C2CCN(C(=O)[C@H]3CCC(=O)N3)CC2)ncc1Cl)c1ccc(Cl)cc1Cl] | | + | 496.1 |
| 1.161 | 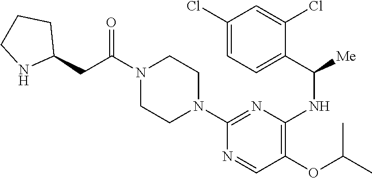 [SMILES: CC(C)Oc1cnc(N2CCN(C(=O)C[C@@H]3CCCN3)CC2)nc1N[C@H](C)c1ccc(Cl)cc1Cl] | | +++ | 521.3 |
| 1.162 | 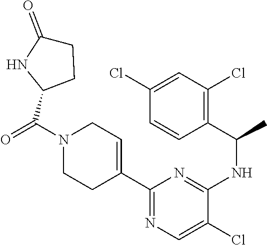 [SMILES: C[C@@H](Nc1nc(C2=CCN(C(=O)[C@H]3CCC(=O)N3)CC2)ncc1Cl)c1ccc(Cl)cc1Cl] | | +++ | 494.1 |
| 1.163 | 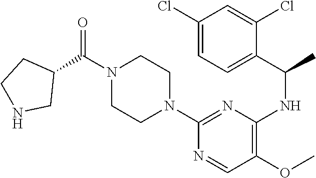 [SMILES: COc1cnc(N2CCN(C(=O)[C@H]3CCNC3)CC2)nc1N[C@H](C)c1ccc(Cl)cc1Cl] | | + ++ | 479.2 |
| 1.164 | 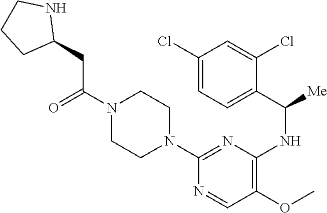 [SMILES: COc1cnc(N2CCN(C(=O)C[C@H]3CCCN3)CC2)nc1N[C@H](C)c1ccc(Cl)cc1Cl] | | +++ | 493.2 |
| 1.165 | 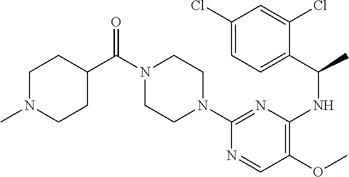 | | +++ | 507.2 |
| 1.166 | 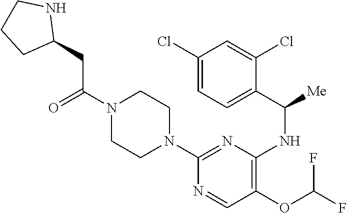 [SMILES: C[C@@H](Nc1nc(N2CCN(C(=O)C[C@H]3CCCN3)CC2)ncc1OC(F)F)c1ccc(Cl)cc1Cl] | | +++ | 529.2 |
| 1.167 | 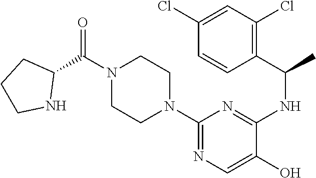 [SMILES: C[C@@H](Nc1nc(N2CCN(C(=O)[C@H]3CCCN3)CC2)ncc1O)c1ccc(Cl)cc1Cl] | | +++ | 465.1 |
| 1.168 | 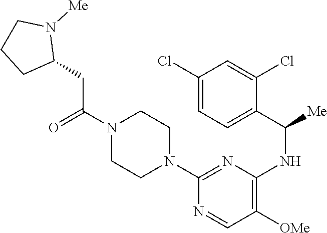 [SMILES: COc1cnc(N2CCN(C(=O)C[C@@H]3CCCN3C)CC2)nc1N[C@H](C)c1ccc(Cl)cc1Cl] | | +++ | 507.3 |
| 1.169 | 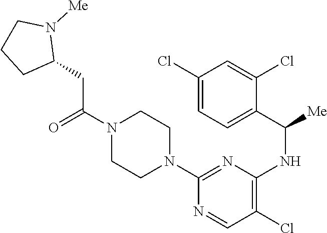 [SMILES: C[C@@H](Nc1nc(N2CCN(C(=O)C[C@@H]3CCCN3C)CC2)ncc1Cl)c1ccc(Cl)cc1Cl] | | +++ | 511.2 |
| 1.170 | 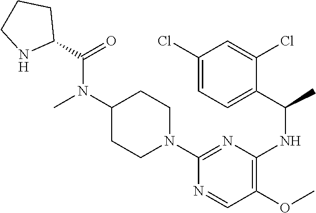 [SMILES: COc1cnc(N2CCC(N(C)C(=O)[C@H]3CCCN3)CC2)nc1N[C@H](C)c1ccc(Cl)cc1Cl] | | +++ | 507.2 |
| 1.171 | 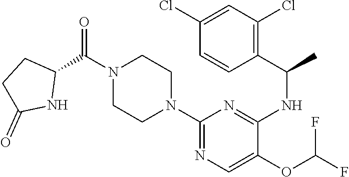 [SMILES: C[C@@H](Nc1nc(N2CCN(C(=O)[C@H]3CCC(=O)N3)CC2)ncc1OC(F)F)c1ccc(Cl)cc1Cl] | | +++ | 529.2 |
| 1.172 | 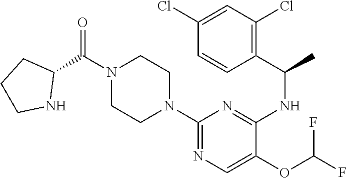 [SMILES: C[C@@H](Nc1nc(N2CCN(C(=O)[C@H]3CCCN3)CC2)ncc1OC(F)F)c1ccc(Cl)cc1Cl] | | +++ | 515.2 |
| 1.173 | 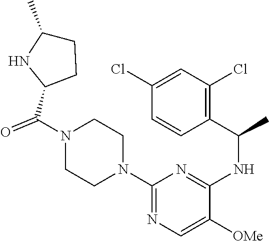 [SMILES: COc1cnc(N2CCN(C(=O)[C@H]3CC[C@@H](C)N3)CC2)nc1N[C@H](C)c1ccc(Cl)cc1Cl] | | +++ | 493.2 |
| 1.174 | 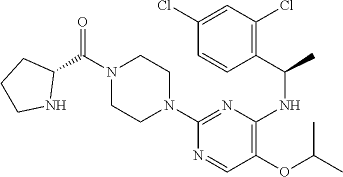 [SMILES: CC(C)Oc1cnc(N2CCN(C(=O)[C@H]3CCCN3)CC2)nc1N[C@H](C)c1ccc(Cl)cc1Cl] | +++ | 507.2 | |
| 1.175 | 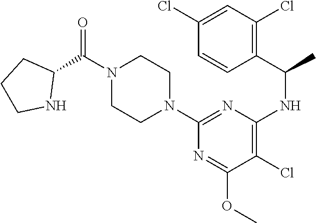 [SMILES: COc1nc(N2CCN(C(=O)[C@H]3CCCN3)CC2)nc(N[C@H](C)c2ccc(Cl)cc2Cl)c1Cl] | +++ | 513.2 | |
| 1.176 | 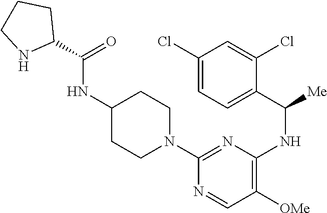 | +++ | 493.2 | |
| 1.177 | 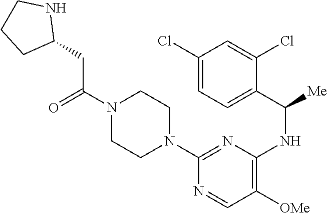 [SMILES: COc1cnc(N2CCN(C(=O)C[C@@H]3CCCN3)CC2)nc1N[C@H](C)c1ccc(Cl)cc1Cl] | +++ | 493.2 | |
| 1.178 | 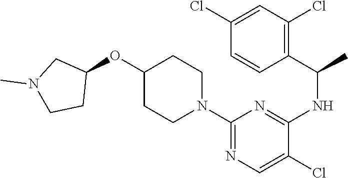 [SMILES: C[C@@H](Nc1nc(N2CCC(O[C@H]3CCN(C)C3)CC2)ncc1Cl)c1ccc(Cl)cc1Cl] | +++ | 484.1 | |
| 1.179 | 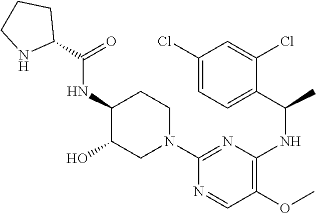 [SMILES: COc1cnc(N2CC[C@H](NC(=O)[C@H]3CCCN3)[C@@H](O)C2)nc1N[C@H](C)c1ccc(Cl)cc1Cl] | +++ | 509.2 | |
| 1.180 | 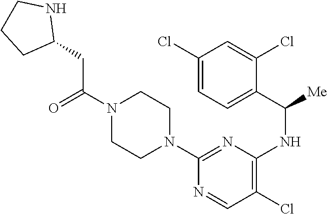 [SMILES: C[C@@H](Nc1nc(N2CCN(C(=O)C[C@@H]3CCCN3)CC2)ncc1Cl)c1ccc(Cl)cc1Cl] | | +++ | 497.1 |
| 1.181 | 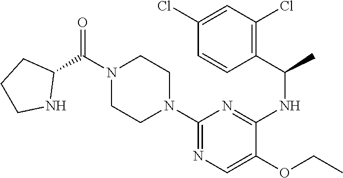 [SMILES: CCOc1cnc(N2CCN(C(=O)[C@H]3CCCN3)CC2)nc1N[C@H](C)c1ccc(Cl)cc1Cl] | | +++ | 493.2 |
| 1.182 | 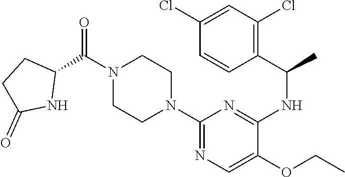 [SMILES: CCOc1cnc(N2CCN(C(=O)[C@H]3CCC(=O)N3)CC2)nc1N[C@H](C)c1ccc(Cl)cc1Cl] | | +++ | 507.2 |
| 1.183 | 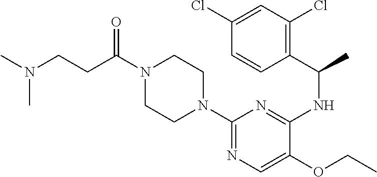 [SMILES: CCOc1cnc(N2CCN(C(=O)CCN(C)C)CC2)nc1N[C@H](C)c1ccc(Cl)cc1Cl] | | +++ | 495.2 |
| 1.184 | 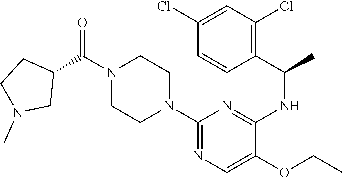 [SMILES: CCOc1cnc(N2CCN(C(=O)[C@H]3CCN(C)C3)CC2)nc1N[C@H](C)c1ccc(Cl)cc1Cl] | | +++ | 507.3 |
| 1.185 | 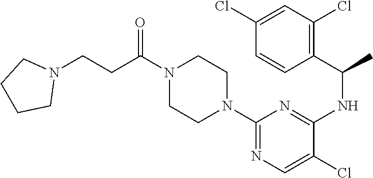 [SMILES: C[C@@H](Nc1nc(N2CCN(C(=O)CCN3CCCC3)CC2)ncc1Cl)c1ccc(Cl)cc1Cl] | | +++ | 511.2 |
| 1.186 | 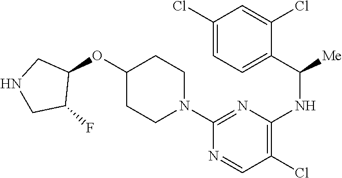 [SMILES: C[C@@H](Nc1nc(N2CCC(O[C@@H]3CNC[C@H]3F)CC2)ncc1Cl)c1ccc(Cl)cc1Cl] | | +++ | 488.1 |
| 1.187 | 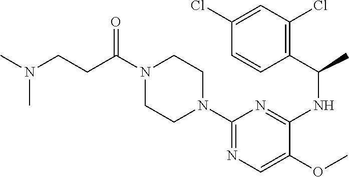 [SMILES: COc1cnc(N2CCN(C(=O)CCN(C)C)CC2)nc1N[C@H](C)c1ccc(Cl)cc1Cl] | | +++ | 481.2 |
| 1.188 | 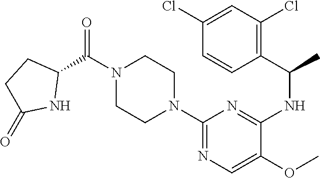 [SMILES: COc1cnc(N2CCN(C(=O)[C@H]3CCC(=O)N3)CC2)nc1N[C@H](C)c1ccc(Cl)cc1Cl] | | +++ | 493.1 |
| 1.189 | 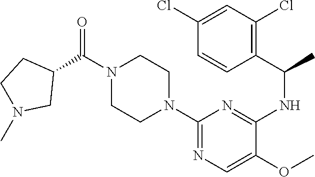 [SMILES: COc1cnc(N2CCN(C(=O)[C@H]3CCN(C)C3)CC2)nc1N[C@H](C)c1ccc(Cl)cc1Cl] | | +++ | 493.2 |
| 1.190 | 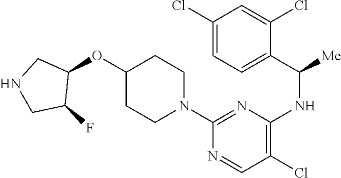 [SMILES: C[C@@H](Nc1nc(N2CCC(O[C@@H]3CNC[C@@H]3F)CC2)ncc1Cl)c1ccc(Cl)cc1Cl] | | +++ | 488.1 |
| 1.191 | 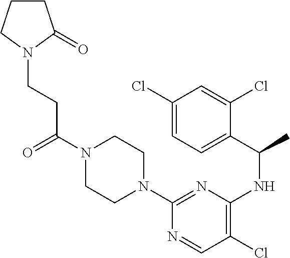 [SMILES: C[C@@H](Nc1nc(N2CCN(C(=O)CCN3CCCC3=O)CC2)ncc1Cl)c1ccc(Cl)cc1Cl] | | ++ | 525.2 |
| 1.192 | 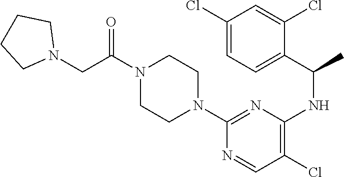 [SMILES: C[C@@H](Nc1nc(N2CCN(C(=O)CN3CCCC3)CC2)ncc1Cl)c1ccc(Cl)cc1Cl] | | +++ | 497.2 |
| 1.193 | 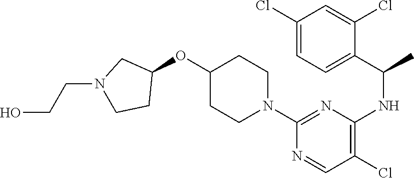 [SMILES: C[C@@H](Nc1nc(N2CCC(O[C@H]3CCN(CCO)C3)CC2)ncc1Cl)c1ccc(Cl)cc1Cl] | | +++ | 514.2 |
| 1.194 | 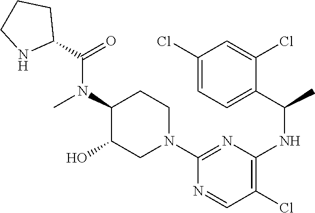 [SMILES: C[C@@H](Nc1nc(N2CC[C@H](N(C)C(=O)[C@H]3CCCN3)[C@@H](O)C2)ncc1Cl)c1ccc(Cl)cc1Cl] | | ++ | 527.1 |
| 1.195 | 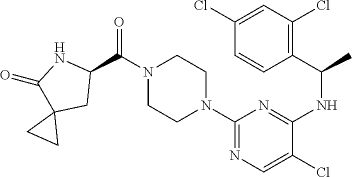 [SMILES: C[C@@H](Nc1nc(N2CCN(C(=O)[C@H]3CC4(CC4)C(=O)N3)CC2)ncc1Cl)c1ccc(Cl)cc1Cl] | | ++ | 523.1 |
| 1.196 | 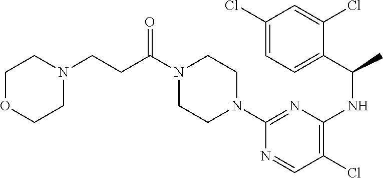 [SMILES: C[C@@H](Nc1nc(N2CCN(C(=O)CCN3CCOCC3)CC2)ncc1Cl)c1ccc(Cl)cc1Cl] | | ++ | 527.2 |
| 1.197 | 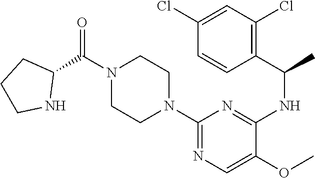 [SMILES: COc1cnc(N2CCN(C(=O)[C@H]3CCCN3)CC2)nc1N[C@H](C)c1ccc(Cl)cc1Cl] | | +++ | 479.3 |
| 1.198 | 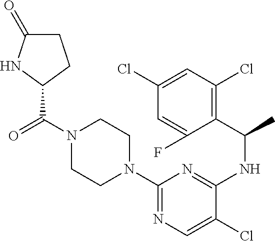 [SMILES: C[C@@H](Nc1nc(N2CCN(C(=O)[C@H]3CCC(=O)N3)CC2)ncc1Cl)c1c(F)cc(Cl)cc1Cl] | | +++ | 515.1 |
| 1.199 | 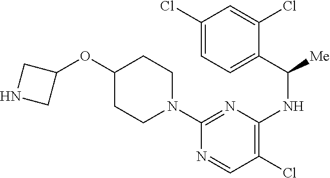 [SMILES: C[C@@H](Nc1nc(N2CCC(OC3CNC3)CC2)ncc1Cl)c1ccc(Cl)cc1Cl] | | ++ | 456.1 |
| 1.200 | 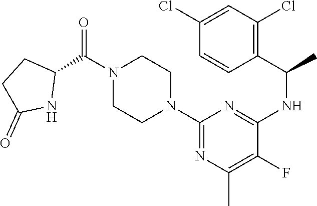 [SMILES: Cc1nc(N2CCN(C(=O)[C@H]3CCC(=O)N3)CC2)nc(N[C@H](C)c2ccc(Cl)cc2Cl)c1F] | | +++ | 511.1 |
| 1.201 | 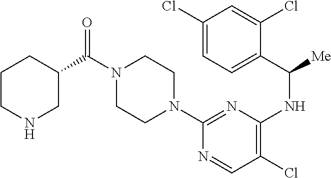 [SMILES: C[C@@H](Nc1nc(N2CCN(C(=O)[C@H]3CCCNC3)CC2)ncc1Cl)c1ccc(Cl)cc1Cl] | | +++ | 497.1 |
| 1.202 | 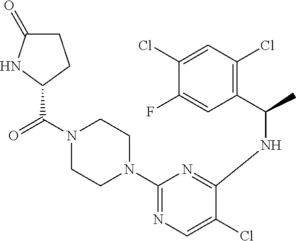 [SMILES: C[C@@H](Nc1nc(N2CCN(C(=O)[C@H]3CCC(=O)N3)CC2)ncc1Cl)c1cc(F)c(Cl)cc1Cl] | | +++ | 515.1 |
| 1.203 | 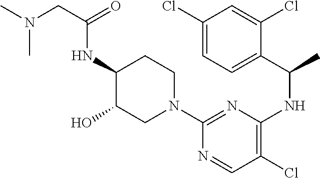 [SMILES: C[C@@H](Nc1nc(N2CC[C@H](NC(=O)CN(C)C)[C@@H](O)C2)ncc1Cl)c1ccc(Cl)cc1Cl] | | ++ | 501.2 |
| 1.204 | 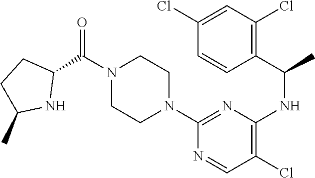 [SMILES: C[C@H]1CC[C@H](C(=O)N2CCN(c3ncc(Cl)c(N[C@H](C)c4ccc(Cl)cc4Cl)n3)CC2)N1] | | +++ | 497.1 |
| 1.205 | 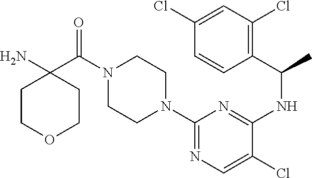 [SMILES: C[C@@H](Nc1nc(N2CCN(C(=O)C3(N)CCOCC3)CC2)ncc1Cl)c1ccc(Cl)cc1Cl] | | ++ | 513.1 |
| 1.206 | 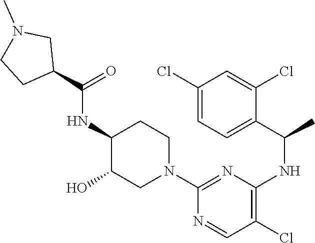 [SMILES: C[C@@H](Nc1nc(N2CC[C@H](NC(=O)[C@H]3CCN(C)C3)[C@@H](O)C2)ncc1Cl)c1ccc(Cl)cc1Cl] | | +++ | 527.2 |
| 1.207 | 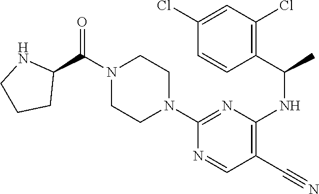 [SMILES: C[C@@H](Nc1nc(N2CCN(C(=O)[C@H]3CCCN3)CC2)ncc1C#N)c1ccc(Cl)cc1Cl] | | ++ | 475.2 |
| 1.208 | 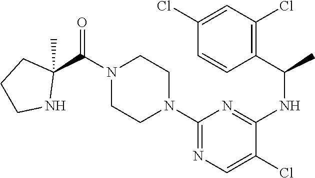 [SMILES: C[C@@H](Nc1nc(N2CCN(C(=O)[C@]3(C)CCCN3)CC2)ncc1Cl)c1ccc(Cl)cc1Cl] | | +++ | 497.1 |
| 1.209 | 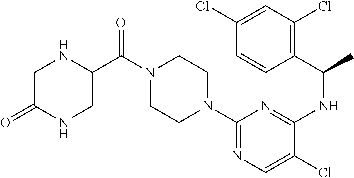 [SMILES: C[C@@H](Nc1nc(N2CCN(C(=O)C3CNC(=O)CN3)CC2)ncc1Cl)c1ccc(Cl)cc1Cl] | | ++ | 512.1 |
| 1.210 | 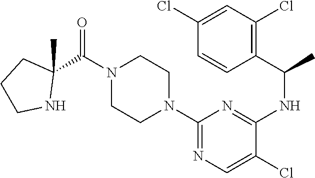 [SMILES: C[C@@H](Nc1nc(N2CCN(C(=O)[C@@]3(C)CCCN3)CC2)ncc1Cl)c1ccc(Cl)cc1Cl] | | +++ | 497.2 |
| 1.211 | 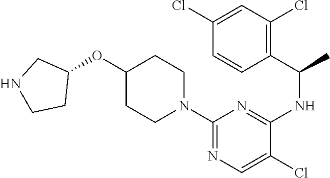 [SMILES: C[C@@H](Nc1nc(N2CCC(O[C@@H]3CCNC3)CC2)ncc1Cl)c1ccc(Cl)cc1Cl] | | +++ | 470.1 |
| 1.212 | 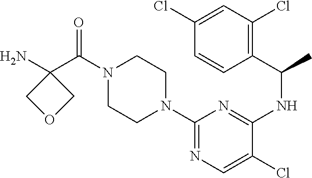 [SMILES: C[C@@H](Nc1nc(N2CCN(C(=O)C3(N)COC3)CC2)ncc1Cl)c1ccc(Cl)cc1Cl] | | + | 485.1 |
| 1.213 | 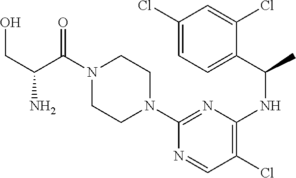 [SMILES: C[C@@H](Nc1nc(N2CCN(C(=O)[C@H](N)CO)CC2)ncc1Cl)c1ccc(Cl)cc1Cl] | | ++ | 473.1 |
| 1.214 | 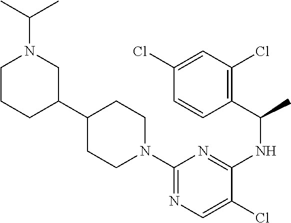 [SMILES: CC(C)N1CCCC(C2CCN(c3ncc(Cl)c(N[C@H](C)c4ccc(Cl)cc4Cl)n3)CC2)C1] | | +++ | 510.2 |
| 1.215 | 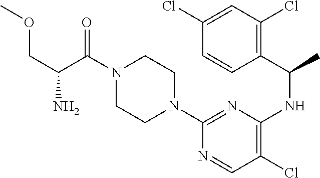 [SMILES: COC[C@@H](N)C(=O)N1CCN(c2ncc(Cl)c(N[C@H](C)c3ccc(Cl)cc3Cl)n2)CC1] | | ++ | 487.1 |
| 1.216 | 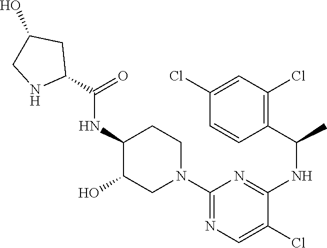 [SMILES: C[C@@H](Nc1nc(N2CC[C@H](NC(=O)[C@H]3C[C@@H](O)CN3)[C@@H](O)C2)ncc1Cl)c1ccc(Cl)cc1Cl] | | ++ | 529.2 |
| 1.217 | 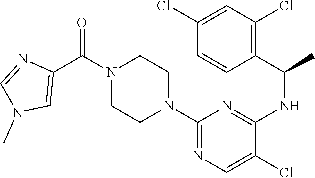 [SMILES: C[C@@H](Nc1nc(N2CCN(C(=O)c3cn(C)cn3)CC2)ncc1Cl)c1ccc(Cl)cc1Cl] | | ++ | 494.1 |
| 1.218 | 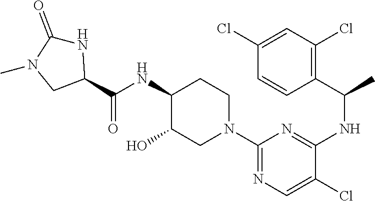 [SMILES: C[C@@H](Nc1nc(N2CC[C@H](NC(=O)[C@H]3CN(C)C(=O)N3)[C@@H](O)C2)ncc1Cl)c1ccc(Cl)cc1Cl] | | + | 542.2 |
| 1.219 | 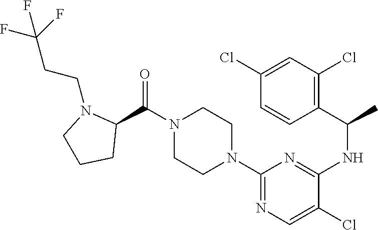 [SMILES: C[C@@H](Nc1nc(N2CCN(C(=O)[C@H]3CCCN3CCC(F)(F)F)CC2)ncc1Cl)c1ccc(Cl)cc1Cl] | | ++ | 579.2 |
| 1.220 | 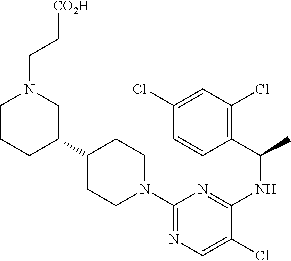 [SMILES: C[C@@H](Nc1nc(N2CCC([C@@H]3CCCN(CCC(=O)O)C3)CC2)ncc1Cl)c1ccc(Cl)cc1Cl] | | +++ | 540.2 |
| 1.221 | 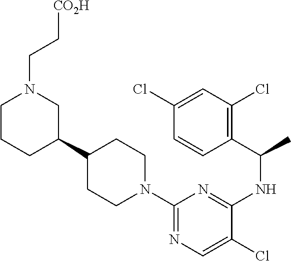 [SMILES: C[C@@H](Nc1nc(N2CCC([C@H]3CCCN(CCC(=O)O)C3)CC2)ncc1Cl)c1ccc(Cl)cc1Cl] | | +++ | 540.2 |
| 1.222 | 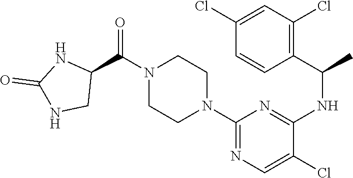 [SMILES: C[C@@H](Nc1nc(N2CCN(C(=O)[C@H]3CNC(=O)N3)CC2)ncc1Cl)c1ccc(Cl)cc1Cl] | | +++ | 499.1 |
| 1.223 | 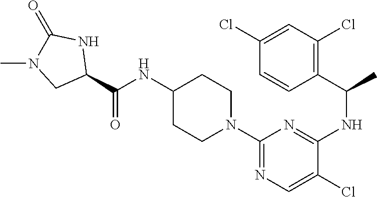 [SMILES: C[C@@H](Nc1nc(N2CCC(NC(=O)[C@H]3CN(C)C(=O)N3)CC2)ncc1Cl)c1ccc(Cl)cc1Cl] | | + | 527.2 |
| 1.224 | 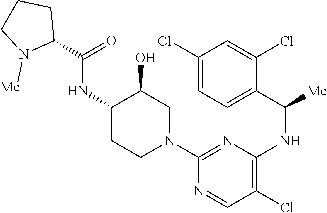 [SMILES: C[C@@H](Nc1nc(N2CC[C@H](NC(=O)[C@H]3CCCN3C)[C@@H](O)C2)ncc1Cl)c1ccc(Cl)cc1Cl] | | +++ | 527.2 |
| 1.225 | 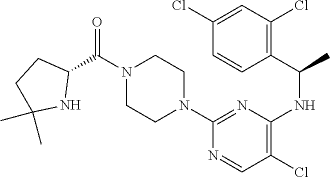 [SMILES: C[C@@H](Nc1nc(N2CCN(C(=O)[C@H]3CCC(C)(C)N3)CC2)ncc1Cl)c1ccc(Cl)cc1Cl] | | +++ | 511.2 |
| 1.226 | 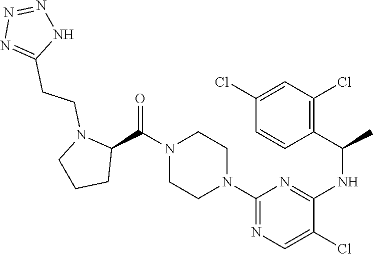 [SMILES: C[C@@H](Nc1nc(N2CCN(C(=O)[C@H]3CCCN3CCc3nnn[nH]3)CC2)ncc1Cl)c1ccc(Cl)cc1Cl] | | +++ | 522.2 |
| 1.227 | 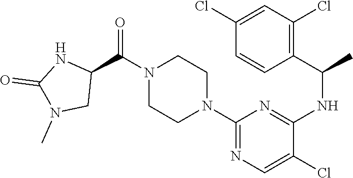 [SMILES: C[C@@H](Nc1nc(N2CCN(C(=O)[C@H]3CN(C)C(=O)N3)CC2)ncc1Cl)c1ccc(Cl)cc1Cl] | | +++ | 512.1 |
| 1.228 | 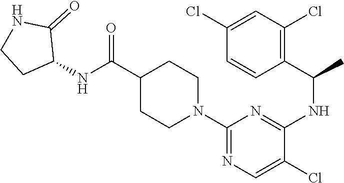 [SMILES: C[C@@H](Nc1nc(N2CCC(C(=O)N[C@@H]3CCNC3=O)CC2)ncc1Cl)c1ccc(Cl)cc1Cl] | | ++ | 511.1 |
| 1.229 | 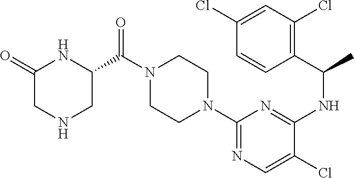 [SMILES: C[C@@H](Nc1nc(N2CCN(C(=O)[C@@H]3CNCC(=O)N3)CC2)ncc1Cl)c1ccc(Cl)cc1Cl] | | + | 512.1 |
| 1.230 | 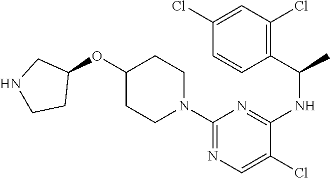 [SMILES: C[C@@H](Nc1nc(N2CCC(O[C@H]3CCNC3)CC2)ncc1Cl)c1ccc(Cl)cc1Cl] | | +++ | 470.2 |
| 1.231 | 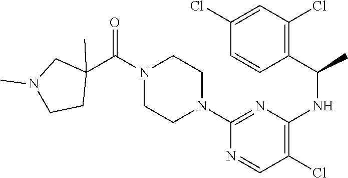 [SMILES: C[C@@H](Nc1nc(N2CCN(C(=O)C3(C)CCN(C)C3)CC2)ncc1Cl)c1ccc(Cl)cc1Cl] | | +++ | 511.1 |
| 1.232 | 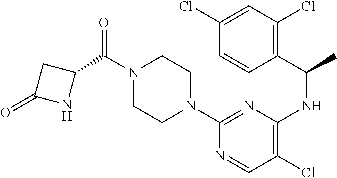 [SMILES: C[C@@H](Nc1nc(N2CCN(C(=O)[C@H]3CC(=O)N3)CC2)ncc1Cl)c1ccc(Cl)cc1Cl] | | ++ | 483.1 |
| 1.233 | 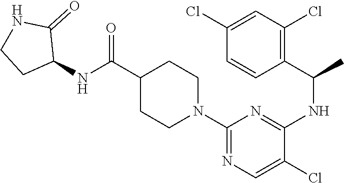 [SMILES: C[C@@H](Nc1nc(N2CCC(C(=O)N[C@H]3CCNC3=O)CC2)ncc1Cl)c1ccc(Cl)cc1Cl] | | + | 511.1 |
| 1.234 | 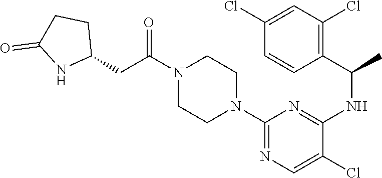 [SMILES: C[C@@H](Nc1nc(N2CCN(C(=O)C[C@H]3CCC(=O)N3)CC2)ncc1Cl)c1ccc(Cl)cc1Cl] | | ++ | 511.1 |
| 1.235 | 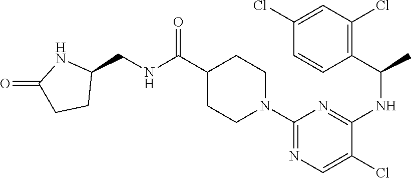 [SMILES: C[C@@H](Nc1nc(N2CCC(C(=O)NC[C@H]3CCC(=O)N3)CC2)ncc1Cl)c1ccc(Cl)cc1Cl] | | + | 525.2 |
| 1.236 | 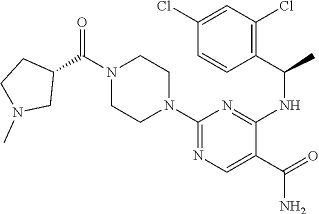 [SMILES: C[C@@H](Nc1nc(N2CCN(C(=O)[C@H]3CCN(C)C3)CC2)ncc1C(N)=O)c1ccc(Cl)cc1Cl] | | +++ | 506.2 |
| 1.237 | 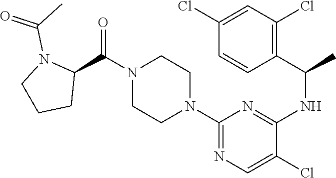 [SMILES: CC(=O)N1CCC[C@@H]1C(=O)N1CCN(c2ncc(Cl)c(N[C@H](C)c3ccc(Cl)cc3Cl)n2)CC1] | | ++ | 526.2 |
| 1.238 | 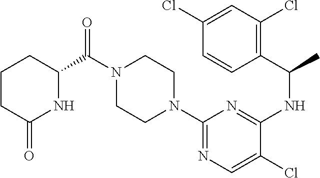 [SMILES: C[C@@H](Nc1nc(N2CCN(C(=O)[C@H]3CCCC(=O)N3)CC2)ncc1Cl)c1ccc(Cl)cc1Cl] | | +++ | 511.1 |
| 1.239 | 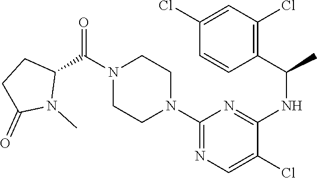 [SMILES: C[C@@H](Nc1nc(N2CCN(C(=O)[C@H]3CCC(=O)N3C)CC2)ncc1Cl)c1ccc(Cl)cc1Cl] | | +++ | 511.1 |
| 1.240 | 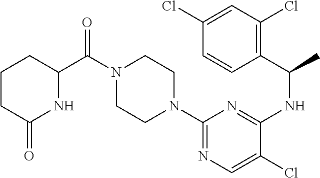 [SMILES: C[C@@H](Nc1nc(N2CCN(C(=O)C3CCCC(=O)N3)CC2)ncc1Cl)c1ccc(Cl)cc1Cl] | | +++ | 511.1 |
| 1.241 | 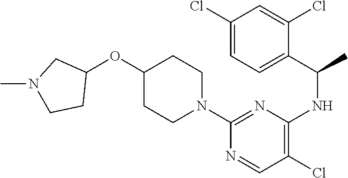 [SMILES: C[C@@H](Nc1nc(N2CCC(OC3CCN(C)C3)CC2)ncc1Cl)c1ccc(Cl)cc1Cl] | | +++ | 484.1 |
| 1.242 | 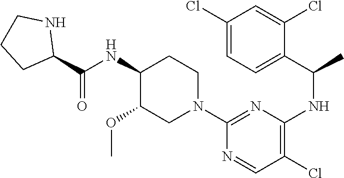 [SMILES: CO[C@H]1CN(c2ncc(Cl)c(N[C@H](C)c3ccc(Cl)cc3Cl)n2)CC[C@@H]1NC(=O)[C@H]1CCCN1] | | +++ | 527.1 |
| 1.243 | 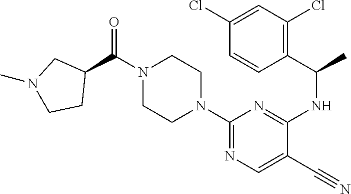 [SMILES: C[C@@H](Nc1nc(N2CCN(C(=O)[C@H]3CCN(C)C3)CC2)ncc1C#N)c1ccc(Cl)cc1Cl] | | +++ | 488.1 |
| 1.244 | 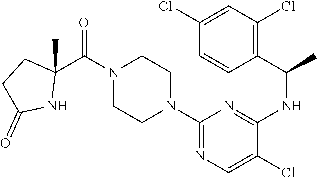 [SMILES: C[C@@H](Nc1nc(N2CCN(C(=O)[C@@]3(C)CCC(=O)N3)CC2)ncc1Cl)c1ccc(Cl)cc1Cl] | | + | 511.1 |
| 1.245 | 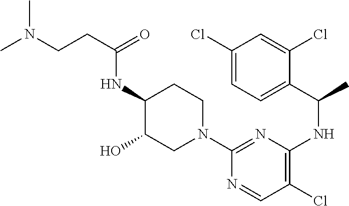 [SMILES: C[C@@H](Nc1nc(N2CC[C@H](NC(=O)CCN(C)C)[C@@H](O)C2)ncc1Cl)c1ccc(Cl)cc1Cl] | | +++ | 515.2 |
| 1.246 | 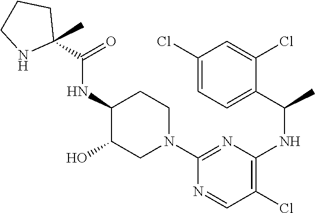 [SMILES: C[C@@H](Nc1nc(N2CC[C@H](NC(=O)[C@@]3(C)CCCN3)[C@@H](O)C2)ncc1Cl)c1ccc(Cl)cc1Cl] | | ++ | 527.2 |
| 1.247 | 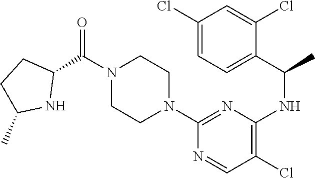 [SMILES: C[C@@H]1CC[C@H](C(=O)N2CCN(c3ncc(Cl)c(N[C@H](C)c4ccc(Cl)cc4Cl)n3)CC2)N1] | | +++ | 497.1 |
| 1.248 | 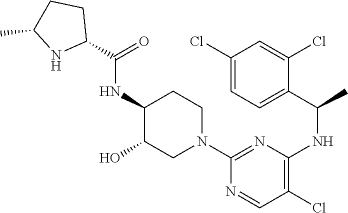 [SMILES: C[C@@H]1CC[C@H](C(=O)N[C@H]2CCN(c3ncc(Cl)c(N[C@H](C)c4ccc(Cl)cc4Cl)n3)C[C@@H]2O)N1] | | +++ | 527.3 |
| 1.249 | 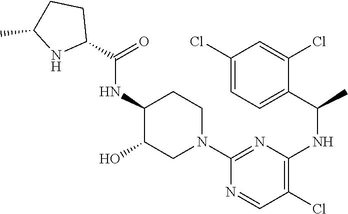 [SMILES: C[C@@H]1CC[C@H](C(=O)N[C@H]2CCN(c3ncc(Cl)c(N[C@H](C)c4ccc(Cl)cc4Cl)n3)C[C@@H]2O)N1] | | +++ | 527.2 |
| 1.250 | 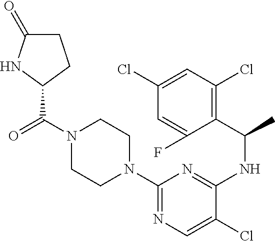 [SMILES: C[C@@H](Nc1nc(N2CCN(C(=O)[C@H]3CCC(=O)N3)CC2)ncc1Cl)c1c(F)cc(Cl)cc1Cl] | +++ | +++ | 515.1 |
| 1.251 | 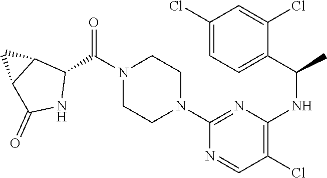 [SMILES: C[C@@H](Nc1nc(N2CCN(C(=O)[C@@H]3NC(=O)[C@@H]4C[C@@H]43)CC2)ncc1Cl)c1ccc(Cl)cc1Cl] | +++ | +++ | 509.1 |
| 1.252 | 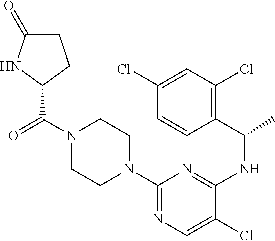 [SMILES: C[C@H](Nc1nc(N2CCN(C(=O)[C@H]3CCC(=O)N3)CC2)ncc1Cl)c1ccc(Cl)cc1Cl] | + | + | 497.1 |
| 1.253 | 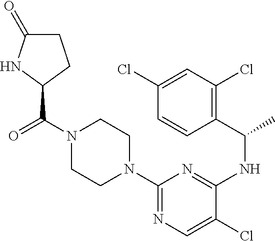 [SMILES: C[C@H](Nc1nc(N2CCN(C(=O)[C@@H]3CCC(=O)N3)CC2)ncc1Cl)c1ccc(Cl)cc1Cl] | + | + | 497.1 |
| 1.254 | 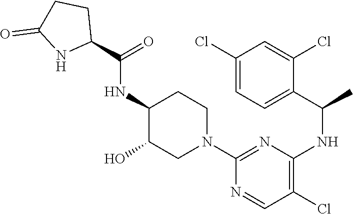 [SMILES: C[C@@H](Nc1nc(N2CC[C@H](NC(=O)[C@@H]3CCC(=O)N3)[C@@H](O)C2)ncc1Cl)c1ccc(Cl)cc1Cl] | + | +++ | 527.2 |
| 1.255 | 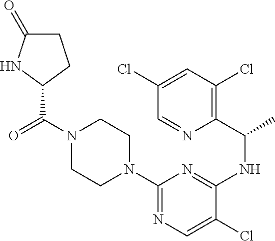 [SMILES: C[C@H](Nc1nc(N2CCN(C(=O)[C@H]3CCC(=O)N3)CC2)ncc1Cl)c1ncc(Cl)cc1Cl] | | +++ | 498.1 |
| 1.256 | 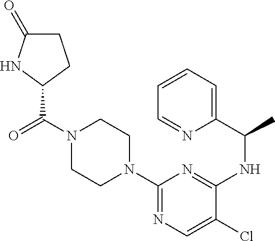 [SMILES: C[C@@H](Nc1nc(N2CCN(C(=O)[C@H]3CCC(=O)N3)CC2)ncc1Cl)c1ccccn1] | + | + | 429.1 |
| 1.257 | 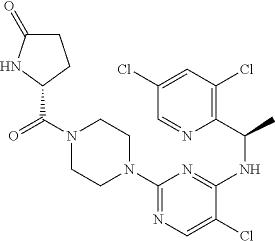 [SMILES: C[C@@H](Nc1nc(N2CCN(C(=O)[C@H]3CCC(=O)N3)CC2)ncc1Cl)c1ncc(Cl)cc1Cl] | + | + | 529.1 |
| 1.258 | 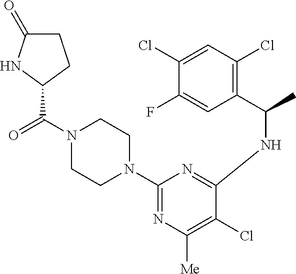 [SMILES: Cc1nc(N2CCN(C(=O)[C@H]3CCC(=O)N3)CC2)nc(N[C@H](C)c2cc(F)c(Cl)cc2Cl)c1Cl] | +++ | +++ | 529.1 |
| 1.259 | 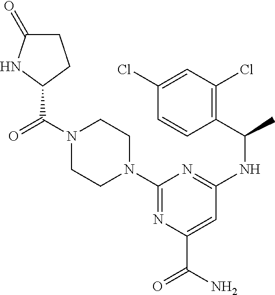 [SMILES: C[C@@H](Nc1cc(C(N)=O)nc(N2CCN(C(=O)[C@H]3CCC(=O)N3)CC2)n1)c1ccc(Cl)cc1Cl] | | +++ | 515.1 |
| 1.260 | 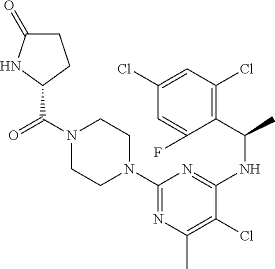 [SMILES: Cc1nc(N2CCN(C(=O)[C@H]3CCC(=O)N3)CC2)nc(N[C@H](C)c2c(F)cc(Cl)cc2Cl)c1Cl] | | +++ | 529.1 |
| 1.261 | 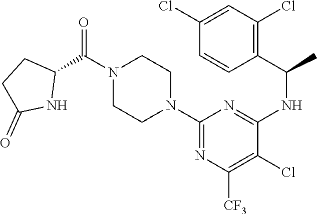 [SMILES: C[C@@H](Nc1nc(N2CCN(C(=O)[C@H]3CCC(=O)N3)CC2)nc(C(F)(F)F)c1Cl)c1ccc(Cl)cc1Cl] | | +++ | 565.1 |

What is claimed is:

1. A compound having formula (I): 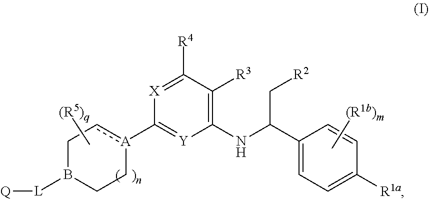
or a pharmaceutically acceptable salt thereof, wherein
R^{1a} is selected from the group consisting of hydrogen, halogen, C₁₋₄ alkyl, C₁₋₄ haloalkyl, —CN, C₁₋₄ alkoxy, and C₁₋₄ haloalkoxy;
m is an integer of from 0 to 4;
each R^{1b} is independently selected from the group consisting of halogen, C₁₋₄ alkyl, C₁₋₄ haloalkyl, —CN, C₁₋₄ alkoxy, and C₁₋₄ haloalkoxy;
R² is selected from the group consisting of H, —OR^{a}, —N(R^{a})₂, C₁₋₄ alkyl, C₁₋₄haloalkyl, and C₁₋₄ hydroxyalkyl;
R³ is selected from the group consisting of hydrogen, C₁₋₄ alkyl, halogen, C₁₋₄haloalkyl, C₁₋₄ hydroxyalkyl, C₃₋₈cycloalkyl, C₁₋₄ alkoxy, C₁₋₄haloalkoxy, C₁₋₄ alkoxy-C₁₋₄ alkyl, —C(O)NH₂, hydroxy, —NH₂, and CN;
each R⁴ is selected from the group consisting of hydrogen, halogen, hydroxy, —CN, C₁₋₄ alkoxy, C₁₋₄ haloalkoxy, —NH₂, C₁₋₄ alkyl, C₁₋₄haloalkyl, C₁₋₄ hydroxyalkyl, C₃₋₈ cycloalkyl, —SO₂Me, and —C(O)NH₂;
n is an integer of from 0-2;
X and Y are each independently N or C(R⁴), and at least one of X and Y is N; [symbol]
is a single or double bond;
A is C, N or C(R^{5a}), provided that when A is N, [symbol] is a single bond;
B is N or C(R^{5b}), and at least one of A and B is N;
q is an integer of from 0 to 4;
each R⁵ is independently selected from the group consisting of C₁₋₄ alkyl, C₁₋₄ alkoxy, —C(O)OH, halogen, hydroxy, C₁₋₄haloalkyl, and C₁₋₄ hydroxyalkyl, or two R⁵ are combined to form a one or two carbon bridge between non-adjacent ring vertices;
R^{5a} is selected from the group consisting of hydrogen, C₁₋₄ alkyl, C₁₋₄ haloalkyl, and C₁₋₄ hydroxyalkyl;
R^{5b} is selected from the group consisting of hydrogen, C₁₋₄ alkyl, and C₁₋₄ hydroxyalkyl;
L is selected from the group consisting of a bond, —O—, —C(O)—C₀₋₄ alkylene-, —C₁₋₄ alkylene- —C(O)—, —C(O)N(R^{a})—C₀₋₄ alkylene-, —S(O)₂—C₀₋₄ alkylene and —N(R^{a})C(O)—C₀₋₄ alkylene-;
Q is a member selected from the group consisting of:
i) 4- to 7-membered heterocyclyl having from one to three heteroatom ring vertices selected from N, O and S, and which is substituted with 0-4 R^{b};
ii) C₁₋₈ alkyl which is substituted with 0-3 R^{b}; and
iii) 7- to 11-membered spirocyclyl having from zero to three heteroatom ring vertices selected from N, O and S, and which is substituted with 0-4 R^{b};
iv) 5- to 6-membered heteroaryl having from one to three heteroatom ring vertices selected from N, O and S, and which is substituted with 0-4 R^{b};
each R^{a} is independently selected from the group consisting of H and C₁₋₄ alkyl optionally substituted with OH or —C(O)OH; and
each R^{b} is independently selected from the group consisting of hydroxy, halogen, oxo, —C₀₋₄ alkylene-N(R^{a})₂, —CO₂R^{a}, C₁₋₄ alkyl, C₁₋₄alkoxy, C₁₋₄haloalkyl, C₁₋₄ hydroxyalkyl, —C₁₋₄ alkylene-CO₂R^{a}, —C₀₋₄ alkylene-heteroaryl, wherein the heteroaryl has from 5- to 6-ring members and one to four heteroatom ring vertices selected from N, O and S, the heteroaryl is optionally substituted with 1-3 R^{c}, —C₀₋₄ alkylene-C₃₋₈ cycloalkyl optionally substituted with 1-3 R^{c}, —C(O)—C₁₋₄ alkyl, and —C₀₋₄ alkylene-C(O)N(R^{a})₂; and
each R^{c} is independently selected from the group consisting of halogen, C₁₋₄ alkyl, C₁₋₄ haloalkyl, and —C(O)OH.

2. A compound having formula (I): 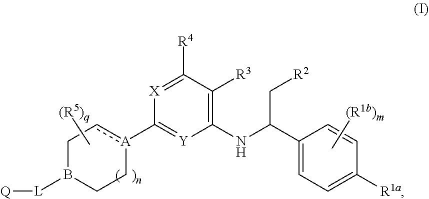
or a pharmaceutically acceptable salt thereof, wherein
R^{1a} is selected from hydrogen, halogen, C₁₋₄ alkyl, C₁₋₄ haloalkyl, —CN, C₁₋₄ alkoxy and C₁₋₄ haloalkoxy;
m is an integer of from 0 to 4;
each R^{1b} is independently selected from the group consisting of halogen, C₁₋₄ alkyl, C₁₋₄ haloalkyl, —CN, C₁₋₄ alkoxy and C₁₋₄ haloalkoxy;
R² is selected from the group consisting of H, —OW, —N(R^{a})₂, C₁₋₄ alkyl, C₁₋₄ haloalkyl, and C₁₋₄ hydroxyalkyl;
R³ is selected from the group consisting of C₁₋₄ alkyl, halogen, CN, and CF₃;
each R⁴ is selected from the group consisting of hydrogen, halogen, —CN, C₁₋₄ alkyl, C₁₋₄ haloalkyl, C₁₋₄ hydroxyalkyl, C₃₋₈ cycloalkyl, —SO₂Me and —C(O)NH₂;
n is 0, 1 or 2;
X and Y are each independently N or C(R⁴), and at least one of X and Y is N; [symbol]
is a single or double bond;
A is C, N or C(R^{5a});
B is N or C(R^{5b}), and at least one of A and B is N;
q is an integer of from 0 to 4;
each R⁵ is independently selected from C₁₋₄ alkyl, hydroxy, C₁₋₄ haloalkyl and C₁₋₄ hydroxyalkyl, or two R⁵ are combined to form a one or two carbon bridge between non-adjacent ring vertices;
R^{5a} is selected from the group consisting of hydrogen, C₁₋₄ alkyl, C₁₋₄ haloalkyl and C₁₋₄ hydroxyalkyl;
R^{5b} is hydrogen and C₁₋₄ alkyl;
L is selected from the group consisting of a bond, —C(O)—, —CH₂C(O)—, —C(O)CH₂—, —C(O)N(R^{a})—, and —N(R^{a})C(O)—;
Q is a member selected from the group consisting of:
i) 4- to 7-membered heterocyclyl having from one to three heteroatom ring vertices selected from N, O and S, and which is substituted with 0-4 R^{b}; and
ii) C₁₋₈ alkyl which is substituted with 0-3 R^{b};
each R^{a} is independently selected from the group consisting of H and C₁₋₄ alkyl;
each R^{b} is independently selected from the group consisting of hydroxy, oxo, —N(R^{a})₂, —CO₂R^{a}, C₁₋₄alkyl, C₁₋₄haloalkyl, C₁₋₄ hydroxyalkyl, C₁₋₄alkylene-CO₂R^{a}, and C₁₋₄ alkylene-N(R^{a})₂.

3. The compound of claim 1 or 2, wherein the first listed moiety in the L group is attached to the ring comprising variable position B.

4. The compound of any of the preceding claims, wherein said compound is optically enriched or optically pure.

5. The compound of any of the preceding claims, wherein n is 1.

6. The compound of any of the preceding claims, wherein R^{1a} and each R^{1b} are halogen.

7. The compound of any of the preceding claims, wherein R² is H or CH₃.

8. The compound of any of the preceding claims, wherein R⁴ is hydrogen.

9. The compound of any of the preceding claims, wherein Q is selected from the group consisting of azetidinyl, pyrrolidinyl, piperidinyl and piperazinyl, each of which is substituted with 0-2 R^{b}.

10. The compound of claim 1 or 2, having the formula (Ia) 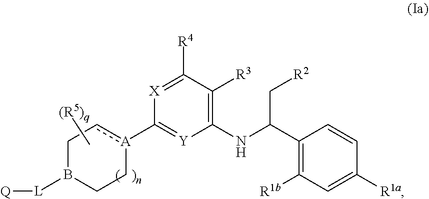
or a pharmaceutically acceptable salt thereof.

11. The compound of claim 10, wherein Q is selected from the group consisting of azetidinyl, pyrrolidinyl, piperidinyl and piperazinyl, each of which is substituted with 0-2 R^{b}.

12. The compound of claim 10, wherein Q is C₁₋₈ alkyl which is substituted with 0-3 R^{b}.

13. The compound of any one of claims 1 to 12, wherein L is selected from the group consisting of a bond, —C(O)—, —CH₂C(O)—, —C(O)CH₂—, —C(O)NH—, —NHC(O)—, —C(O)N(CH₃)—, and —N(CH₃)C(O)—.

14. The compound of claim 1 or 2, having the formula (Ib) 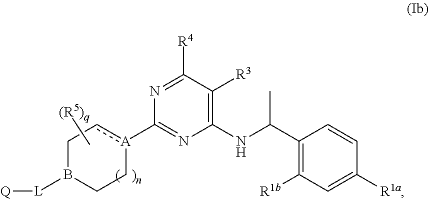
or a pharmaceutically acceptable salt thereof.

15. The compound of claim 14, wherein Q is selected from the group consisting of azetidinyl, pyrrolidinyl, piperidinyl and piperazinyl, each of which is substituted with 0-2 R^{b}.

16. The compound of claim 14, wherein Q is C₁₋₈ alkyl which is substituted with 0-3 R^{b}.

17. The compound of claim 1 or 2, having the formula (Ic1), (Ic2) or (Ic3): 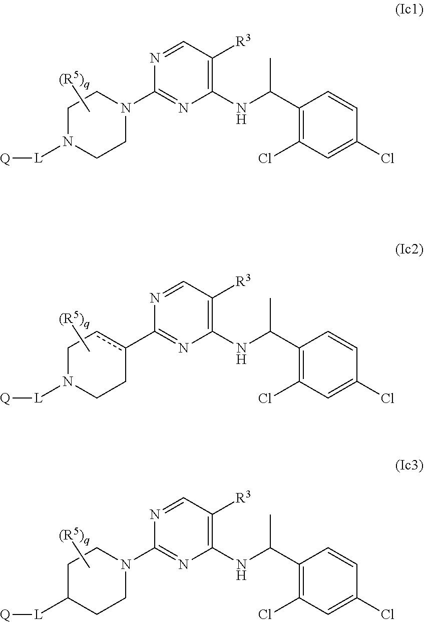
or a pharmaceutically acceptable salt thereof.

18. The compound of claim 17, wherein Q is selected from the group consisting of azetidinyl, pyrrolidinyl, piperidinyl and piperazinyl, each of which is substituted with 0-2 R^{b}.

19. The compound of claim 17, wherein Q is C₁₋₈ alkyl which is substituted with 0-3 R^{b}.

20. The compound of claim 1 or 2, having the formula (Id1), (Id2), or (Id3), 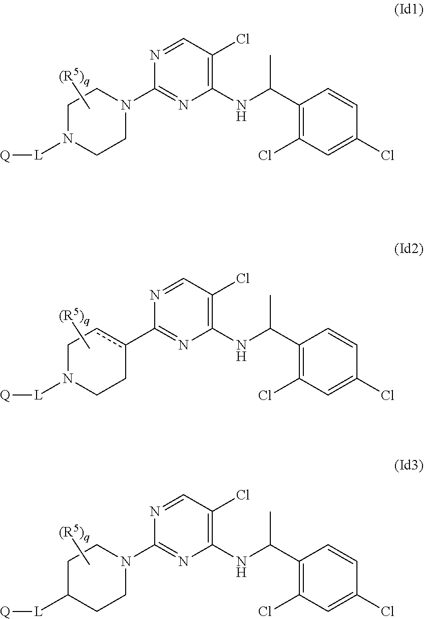
or a pharmaceutically acceptable salt thereof.

21. The compound of claim 20, wherein Q is selected from the group consisting of azetidinyl, pyrrolidinyl, piperidinyl and piperazinyl, each of which is substituted with 0-2 R^{b}.

22. The compound of claim 20, wherein Q is C₁₋₈ alkyl which is substituted with 0-3 R^{b}.

23. The compound of any one of claims 20 to 22, wherein L is selected from the group consisting of a bond, —C(O)—, —CH₂C(O)—, —C(O)CH₂—, —C(O)NH—, —NHC(O)—, —C(O)N(CH₃)—, and —N(CH₃)C(O)—.

24. The compound of claim 1 or 2, having one of the following formulae: 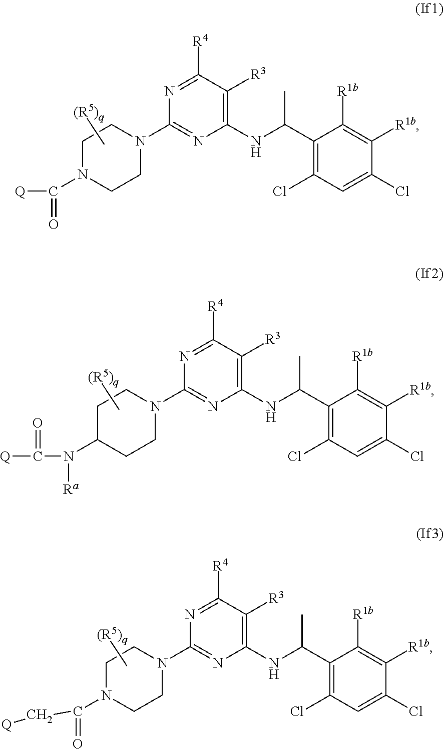
or a pharmaceutically acceptable salt thereof, wherein:
each R^{b} is independently selected from the group consisting of H, Cl or F, provided that at least one R^{b} is H.

25. The compound of any one of claims 1 to 8, 10, 14, 17, 20 or 24, wherein Q is selected from the group consisting of 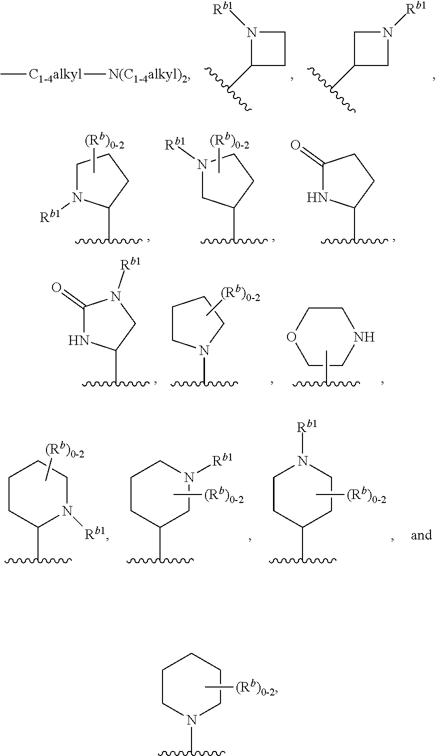
wherein:
each R^{b} is independently selected from the group consisting of C₁₋₄ alkyl, F, Cl, OH, and —N(H)CH₃; and
R^{b1} is selected from the group consisting of H, C₁₋₄ alkyl, C₁₋₄ hydroxyalkyl, C₁₋₄haloalkyl, —C(O)—C₁₋₃ alkyl, —C(O)—O—C₁₋₃ alkyl, —C₁₋₃ alkylene-C(O)OH, and —C(O)NH₂.

26. The compound of claim 1, having one of the following formulae: 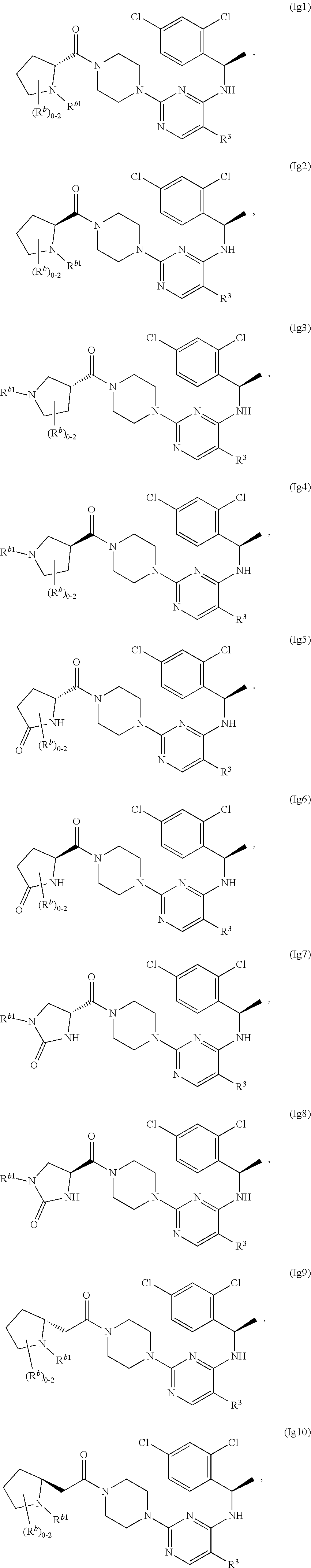
or a pharmaceutically acceptable salt thereof, wherein:
each R^{b} is independently selected from the group consisting of C₁₋₄ alkyl, F, Cl, OH, and —N(H)CH₃; and
R^{b1} is selected from the group consisting of H, C₁₋₄ alkyl, C₁₋₄ hydroxyalkyl, C₁₋₄ haloalkyl, —C(O)—C₁₋₃ alkyl, —C(O)—O—C₁₋₃ alkyl, —C₁₋₃ alkylene-C(O)OH, and —C(O)NH₂.

27. The compound of claim 1, having one of the following formulae: 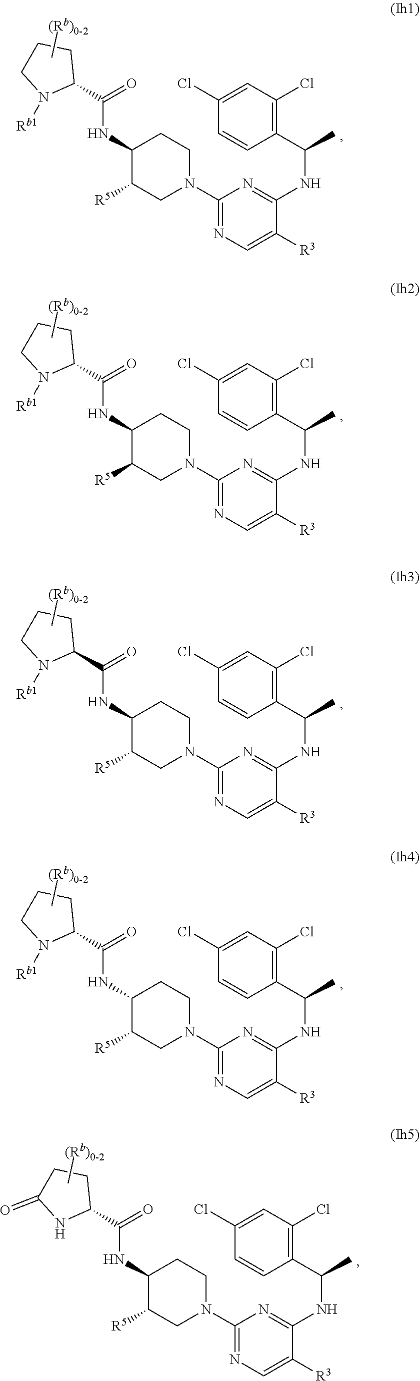 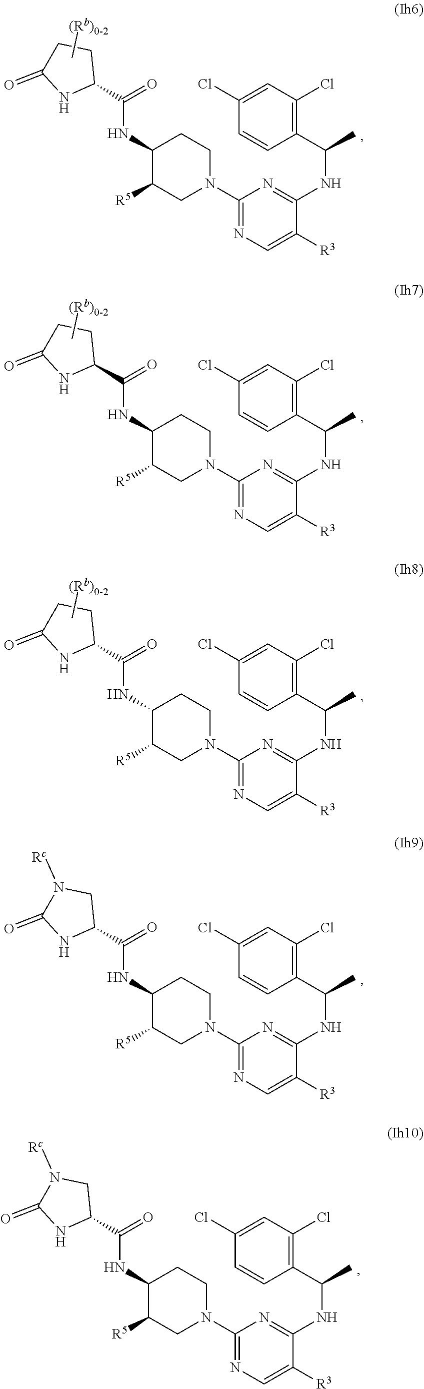 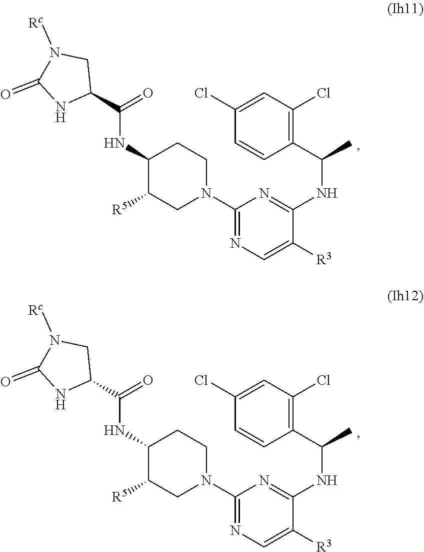
or a pharmaceutically acceptable salt thereof, wherein:
each R^{b} is independently selected from the group consisting of C₁₋₄ alkyl, F, Cl, and OH; and
R⁵ is selected from the group consisting of OH, F and OCH₃.

28. The compound of any of the preceding claims, wherein R³ is selected from the group consisting of C₁₋₄ alkyl, Cl, F, C₁₋₄haloalkyl, C₁₋₄ hydroxyalkyl, cyclopropyl, C₁₋₄ alkoxy, C₁₋₄haloalkoxy, C₁₋₃ alkylene-O—C₁₋₃ alkyl, hydroxy, —NH₂, and CN.

29. The compound of claim 28, wherein R³ is selected from the group consisting of Cl, F, —OCH₃, —OCH₂CH₃, OCH(CH₃)₂, OCF₃, OCHF₂, —C(CH₂)₂OH, and hydroxy.

30. The compound of claim 28, wherein R³ is selected from the group consisting of halogen, —CH₃, —OCH₃, —CH₂OCH₃, —OCH₂CH₃, —OC(H)(CH₃)₂, CN, —NH₂, CF₃, —OCF₃, and —OCHF₂.

31. The compound of claim 28, wherein R³ is Cl.

32. The compound of claim 1, selected from Table 1, or a pharmaceutically acceptable salt thereof.

33. A pharmaceutical composition comprising a pharmaceutically acceptable excipient and a compound of claim 1.

34. A method of treating a disease or condition selected from the group consisting of (1) allergic diseases, (2) inflammatory bowel diseases, (3) vaginitis, (4) psoriasis and inflammatory dermatoses, (5) vasculitis, (6) spondyloarthropathies, (7) scleroderma, (8) asthma and respiratory allergic diseases, (9) autoimmune diseases, (10) graft rejection, (11) other diseases in which undesired inflammatory responses are to be inhibited, and cancer, said method comprising administering to a subject in need thereof a compound of claim 1.

35. A method in accordance with claim 34, wherein said disease or condition is selected from the group consisting of allergic diseases, psoriasis, atopic dermatitis and asthma.

36. A pharmaceutical composition comprising the compound according to any one of claims 1-32, or a pharmaceutically acceptable salt of said compound, and a pharmaceutically acceptable excipient.
